SEQUENCE LISTING (78 sequences)

SEQ ID NO: 1 - protein, 35 aa, Artificial Sequence
  Synthetic formula sequence
CXXXXXXXXDXLXXXCXXCXXXCGXXPXXXCXXXC

SEQ ID NO: 2 - protein, 35 aa, Artificial Sequence
  Synthetic peptide
CXXXXXXXXDXLXXXCXXCXXXCXQHXXXXCXXXC

SEQ ID NO: 3 - protein, 33 aa, Artificial Sequence
  Synthetic peptide
CXXXXXXDXLXXXCXXCXXXCXQHXXXXCXXXC

SEQ ID NO: 4 - protein, 34 aa, Homo sapiens
CRKEQGKFYDHLLRDCISCASICGQHPKQCAYFC

SEQ ID NO: 5 - protein, 39 aa, Homo sapiens
CRKEQGKFYDHLLRDCISCASICGQHPKQCAYFCENKLR

SEQ ID NO: 6 - protein, 34 aa, Mus sp.
CRKEQGRYYDHLLGACVSCDSTCTQHPQQCAHFC

SEQ ID NO: 7 - protein, 39 aa, Mus sp.
CRKEQGRYYDHLLGACVSCDSTCTQHPQQCAHFCEKRPR

SEQ ID NO: 8 - protein, 33 aa, Homo sapiens
CPEEQYWDPLLGTCMSCKTICNHQSQRTCAAFC

SEQ ID NO: 9 - protein, 33 aa, Mus sp.
CPKDQYWDSSRKSCVSCALTCSQRSQRTCTDFC

SEQ ID NO: 10 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 11 - protein, 247 aa, Homo sapiens
MSGLGRSRRGGRSRVDQEERWSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 12 - protein, 265 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRTAPPREG

SEQ ID NO: 13 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 14 - protein, 250 aa, Homo sapiens [sequence omitted]

SEQ ID NO: 15 - protein, 34 aa, Artificial Sequence
  Synthetic peptide
CRKEQGKEYDHLLRDCISCASICGQHPKQCAYFC

SEQ ID NO: 16 - protein, 34 aa, Artificial Sequence
  Synthetic peptide
CRKEQGKSYDHLLRDCISCASICGQHPKQCAYFC

SEQ ID NO: 17 - protein, 34 aa, Artificial Sequence
  Synthetic peptide
CRKEQGKFVDHLLRDCISCASICGQHPKQCAYFC

SEQ ID NO: 18 - protein, 34 aa, Artificial Sequence
  Synthetic peptide
CRKEQGKEVDHLLRDCISCASICGQHPKQCAYFC

SEQ ID NO: 19 - protein, 34 aa, Artificial Sequence
  Synthetic peptide
CRKEQGKSVDHLLRDCISCASICGQHPKQCAYFC

SEQ ID NO: 20 - protein, 32 aa, Artificial Sequence
  Synthetic peptide
CPEEQYWDPLLGTCMSCKTICGQHPKQCAAFC

SEQ ID NO: 21 - protein, 32 aa, Artificial Sequence
  Synthetic peptide
CPEEQEWDPLLGTCMSCKTICGQHPKQCAAFC

SEQ ID NO: 22 - protein, 32 aa, Artificial Sequence
  Synthetic peptide
CPEEQSWDPLLGTCMSCKTICGQHPKQCAAFC

SEQ ID NO: 23 - protein, 32 aa, Artificial Sequence
  Synthetic peptide
CPEEQYVDPLLGTCMSCKTICGQHPKQCAAFC

SEQ ID NO: 24 - protein, 32 aa, Artificial Sequence
  Synthetic peptide
CPEEQEVDPLLGTCMSCKTICGQHPKQCAAFC

SEQ ID NO: 25 - protein, 32 aa, Artificial Sequence
  Synthetic peptide
CPEEQSVDPLLGTCMSCKTICGQHPKQCAAFC

SEQ ID NO: 26 - protein, 34 aa, Artificial Sequence
  Synthetic peptide
CRKEQGKFEDHLLRDCISCASICGQHPKQCAYFC

SEQ ID NO: 27 - protein, 34 aa, Artificial Sequence
  Synthetic peptide
CRKEQGKFYDHLLEDCISCASICGQHPKQCAYFC

SEQ ID NO: 28 - protein, 34 aa, Artificial Sequence
  Synthetic peptide
CRKEQGKFYDHLLWDCISCASICGQHPKQCAYFC

SEQ ID NO: 29 - protein, 34 aa, Artificial Sequence
  Synthetic peptide
CRKEQGKFYDHLLDDCISCASICGQHPKQCAYFC

SEQ ID NO: 30 - protein, 34 aa, Artificial Sequence
  Synthetic peptide
CRKEQGKFYDHLLFDCISCASICGQHPKQCAYFC

SEQ ID NO: 31 - protein, 34 aa, Artificial Sequence
  Synthetic peptide
CRKEQGKFYDHLLMDCISCASICGQHPKQCAYFC

SEQ ID NO: 32 - protein, 34 aa, Artificial Sequence
  Synthetic peptide
CRKEQGKFYDHLLRDCISCASLCGQHPKQCAYFC

SEQ ID NO: 33 - protein, 34 aa, Artificial Sequence
  Synthetic peptide
CRKEQGKFEDHLLEDCISCASICGQHPKQCAYFC

SEQ ID NO: 34 - protein, 34 aa, Artificial Sequence
  Synthetic peptide
CRKEQGKFEDHLLWDCISCASICGQHPKQCAYFC

SEQ ID NO: 35 - protein, 34 aa, Artificial Sequence
  Synthetic peptide
CRKEQGKFEDHLLDDCISCASICGQHPKQCAYFC

SEQ ID NO: 36 - protein, 34 aa, Artificial Sequence
  Synthetic peptide
CRKEQGKFEDHLLFDCISCASICGQHPKQCAYFC

SEQ ID NO: 37 - protein, 34 aa, Artificial Sequence
  Synthetic peptide
CRKEQGKFEDHLLMDCISCASICGQHPKQCAYFC

SEQ ID NO: 38 - protein, 34 aa, Artificial Sequence
  Synthetic peptide
CRKEQGKFYDHLLEDCISCASLCGQHPKQCAYFC

SEQ ID NO: 39 - protein, 34 aa, Artificial Sequence
  Synthetic peptide
CRKEQGKFYDHLLWDCISCASLCGQHPKQCAYFC

SEQ ID NO: 40 - protein, 34 aa, Artificial Sequence
  Synthetic peptide
CRKEQGKFYDHLLDDCISCASLCGQHPKQCAYFC

SEQ ID NO: 41 - protein, 34 aa, Artificial Sequence
  Synthetic peptide
CRKEQGKFYDHLLFDCISCASLCGQHPKQCAYFC

SEQ ID NO: 42 - protein, 34 aa, Artificial Sequence
  Synthetic peptide
CRKEQGKFYDHLLMDCISCASLCGQHPKQCAYFC

SEQ ID NO: 43 - protein, 34 aa, Artificial Sequence
  Synthetic peptide
CRKEQGKFEDHLLEDCISCASLCGQHPKQCAYFC

SEQ ID NO: 44 - protein, 34 aa, Artificial Sequence
  Synthetic peptide
CRKEQGKFEDHLLWDCISCASLCGQHPKQCAYFC

SEQ ID NO: 45 - protein, 34 aa, Artificial Sequence
  Synthetic peptide
CRKEQGKFEDHLLDDCISCASLCGQHPKQCAYFC

SEQ ID NO: 46 - protein, 34 aa, Artificial Sequence
  Synthetic peptide
CRKEQGKFEDHLLFDCISCASLCGQHPKQCAYFC

SEQ ID NO: 47 - protein, 34 aa, Artificial Sequence
  Synthetic peptide
CRKEQGKFEDHLLMDCISCASLCGQHPKQCAYFC

SEQ ID NO: 48 - protein, 32 aa, Artificial Sequence
  Synthetic peptide
CPEEQYEDPLLGTCMSCKTICGQHPKQCAAFC

SEQ ID NO: 49 - protein, 32 aa, Artificial Sequence
  Synthetic peptide
CPEEQYWDPLLETCMSCKTICGQHPKQCAAFC

SEQ ID NO: 50 - protein, 32 aa, Artificial Sequence
  Synthetic peptide
CPEEQYWDPLLWTCMSCKTICGQHPKQCAAFC

SEQ ID NO: 51 - protein, 32 aa, Artificial Sequence
  Synthetic peptide
CPEEQYWDPLLDTCMSCKTICGQHPKQCAAFC

SEQ ID NO: 52 - protein, 32 aa, Artificial Sequence
  Synthetic peptide
CPEEQYWDPLLFTCMSCKTICGQHPKQCAAFC

SEQ ID NO: 53 - protein, 32 aa, Artificial Sequence
  Synthetic peptide
CPEEQYWDPLLMTCMSCKTICGQHPKQCAAFC

SEQ ID NO: 54 - protein, 32 aa, Artificial Sequence
  Synthetic peptide
CPEEQYWDPLLGTCMSCKTLCGQHPKQCAAFC

SEQ ID NO: 55 - protein, 32 aa, Artificial Sequence
  Synthetic peptide
CPEEQYEDPLLETCMSCKTICGQHPKQCAAFC

SEQ ID NO: 56 - protein, 32 aa, Artificial Sequence
  Synthetic peptide
CPEEQYEDPLLWTCMSCKTICGQHPKQCAAFC

SEQ ID NO: 57 - protein, 32 aa, Artificial Sequence
  Synthetic peptide
CPEEQYEDPLLDTCMSCKTICGQHPKQCAAFC

SEQ ID NO: 58 - protein, 32 aa, Artificial Sequence
  Synthetic peptide
CPEEQYEDPLLFTCMSCKTICGQHPKQCAAFC

SEQ ID NO: 59 - protein, 32 aa, Artificial Sequence
  Synthetic peptide
CPEEQYEDPLLMTCMSCKTICGQHPKQCAAFC

SEQ ID NO: 60 - protein, 32 aa, Artificial Sequence
  Synthetic peptide
CPEEQYWDPLLETCMSCKTLCGQHPKQCAAFC

SEQ ID NO: 61 - protein, 32 aa, Artificial Sequence
  Synthetic peptide
CPEEQYWDPLLWTCMSCKTLCGQHPKQCAAFC

SEQ ID NO: 62 - protein, 32 aa, Artificial Sequence
  Synthetic peptide
CPEEQYWDPLLDTCMSCKTLCGQHPKQCAAFC

SEQ ID NO: 63 - protein, 32 aa, Artificial Sequence
  Synthetic peptide
CPEEQYWDPLLFTCMSCKTLCGQHPKQCAAFC

SEQ ID NO: 64 - protein, 32 aa, Artificial Sequence
  Synthetic peptide
CPEEQYWDPLLMTCMSCKTLCGQHPKQCAAFC

SEQ ID NO: 65 - protein, 32 aa, Artificial Sequence
  Synthetic peptide
CPEEQYEDPLLETCMSCKTLCGQHPKQCAAFC

SEQ ID NO: 66 - protein, 32 aa, Artificial Sequence
  Synthetic peptide
CPEEQYEDPLLWTCMSCKTLCGQHPKQCAAFC

SEQ ID NO: 67 - protein, 32 aa, Artificial Sequence
  Synthetic peptide
CPEEQYEDPLLDTCMSCKTLCGQHPKQCAAFC

SEQ ID NO: 68 - protein, 32 aa, Artificial Sequence
  Synthetic peptide
CPEEQYEDPLLFTCMSCKTLCGQHPKQCAAFC

SEQ ID NO: 69 - protein, 32 aa, Artificial Sequence
  Synthetic peptide
CPEEQYEDPLLMTCMSCKTLCGQHPKQCAAFC

SEQ ID NO: 70 - protein, 34 aa, Homo sapiens
CSQNEYFDSLLHACIPCQLRCSSNTPPLTCQRYC

SEQ ID NO: 71 - protein, 309 aa, Mus sp.
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 72 - protein, 4 aa, Artificial Sequence
  Synthetic peptide
XQHX

SEQ ID NO: 73 - protein, 4 aa, Artificial Sequence
  Synthetic peptide
GXXP

SEQ ID NO: 74 - protein, 6 aa, Artificial Sequence
  Synthetic peptide
GXXPXX

SEQ ID NO: 75 - protein, 7 aa, Artificial Sequence
  Synthetic peptide
GXXPXXX

SEQ ID NO: 76 - protein, 6 aa, Artificial Sequence
  Synthetic peptide
XQHXXX

SEQ ID NO: 77 - protein, 7 aa, Artificial Sequence
  Synthetic peptide
XQHXXXX

SEQ ID NO: 78 - protein, 14 aa, Artificial Sequence
  Synthetic peptide
MADPNRFRGKDLGG

(19) United States
(12) Patent Application Publication
(10) Pub. No.: US 2008/0181886 A1
(43) Pub. Date: Jul. 31, 2008

(54) Polypeptides That Bind Baff And/Or April

(75) Inventor: Robert F. Kelley, San Bruno, CA (US)

Correspondence Address: CLARK & ELBING LLP, 101 FEDERAL STREET, BOSTON, MA, 02110, US

(73) Assignee: Genentech, Inc., South San Francisco, CA (US)

(21) Appl. No.: 11/666,781

(22) PCT Filed: Oct. 28, 2005
(86) PCT No.: PCT/US05/39154

§ 371 Date: Sep. 10, 2007

Related U.S. Application Data
(60) Provisional application No. 60/625,341, filed on Nov. 4, 2004.
(60) Provisional application No. 60/673,127, filed on Apr. 19, 2005.

(51) Int. Cl. A61K 39/00; C07K 16/00; C07H 21/04; C12N 15/00; A61K 38/00

(52) U.S. Cl. 424/133.1; 530/350; 530/402; 530/387.3; 536/23.5; 435/320.1; 514/12

(57) ABSTRACT

The present invention relates to novel polypeptides and TACI variants that bind APRIL, novel polypeptides and TACI variants that bind BAFF, nucleic acid molecules encoding the polypeptides, host cells comprising the nucleic acid molecules, compositions comprising the polypeptides or nucleic acid molecules, and methods of using the polypeptides and nucleic acid molecules.

### RELATED APPLICATIONS

This application claims benefit from U.S. Provisional Application Nos. 60/625,341, filed Nov. 4, 2004 and 60/673,127, filed Apr. 19, 2005.

### FIELD OF THE INVENTION

The present invention relates to new polypeptides that bind BAFF, new polypeptides that bind APRIL, and new polypeptides that bind BAFF and APRIL, nucleic acid molecules encoding the polypeptides, compositions comprising them and methods of using the nucleic acid molecule and polypeptides.

### BACKGROUND OF THE INVENTION

TACI, BCMA and BR3 are three members of the TNFR superfamily of receptors (TNFR). All three receptors bind the ligand known as BAFF. TACI and BCMA also bind the ligand APRIL (Marsters et al. (2001) Curr Biol 10, 785-788; Rennert et al. (2000) J Exp Med 192:1677-1684; Thompson et al. (2000) J Exp Med 192:129-135; Wu et al. (2000) J Biol Chem 275:35478-35485). However, only TACI is a high-affinity receptor for both APRIL and BAFF since monovalent BCMA binds BAFF only weakly (Patel et al. (2004) J Biol Chem 279:16727-16735; Pelletier et al. (2003) J Biol Chem 278:33127-33133). TACI functions, at least in part, as a negative regulator of BAFF function as loss of TACI expression results in the over-production of B-cells and causes auto-immunity in mice (Seshasayee et al. (2003) Immunity 18, 279-288; Yan et al. (2001) Curr Biol 11:1547-1552).

BAFF and APRIL are type II transmembrane protein cytokines that have diverse and, at times, opposing effects on various immune cell types including acting as co-stimulatory molecules, apoptotic agents, and growth factors (Locksley et al. (2001) Cell 104:487-501). APRIL, (A PRoliferation-Inducing Ligand) also known as TNSF13A, Tall-2, and TRDL-1, is a TNF ligand that is overexpressed by some tumors and stimulates tumor cell growth (Hahne et al. (1998) J Exp Med 188:1185-1190); however, its function in normal biology is less clear (Medema et al. (2003) Cell Death and Differentiation 10:1121-1125). APRIL is similar in sequence to BAFF, also known as TNFSF13B, BLyS, Tall-1, THANK, and zTNF4. BAFF is essential for the normal development of mature B-cells via signaling through the divergent TNF receptor BR3 (also known as BAFF-R and TNFRSF13C) (Mackay et al., (2003) Annu Rev Immunol 21:231-264; Moore et al. (1999) Science 285:260-263; Schiemann et al. (2001) Science 293:2111-2114; Schneider et al. (1999) J Exp Med 189:1747-1756; Thompson et al. (2001) Science 293:2108-2111); Yan et al. (2001) Curr Biol 11:1547-1552).

The extracellular domain of a typical TNFR contains multiple copies of a ˜40-residue pseudo-repeat, each containing six cysteines, which bind in the monomer-monomer interface of a trimeric ligand (Bodmer et al. (2002) Trends Biochem Sci 27:19-26). Sequence analysis of TACI indicates that it is a member of the TNFR superfamily possessing two cysteine-rich domains (CRD), although the two CRDs of TACI are more similar to each other than is typical in the TNFR family (von Bülow and Bram (1997) Science 278:138-141). BCMA and BR3, in contrast, are unusually small TNFRs because they contain only a single or partial CRD respectively. However, all the APRIL/BAFF receptor CRDs, including both domains of TACI, share a common sequence feature, the so-called DxL motif, which is required for binding to either APRIL or BAFF (Gordon et al. (2003) Biochemistry 42:5977-5983; Kayagaki et al. (2002) Immunity 17:515-524; Kim et al. (2003) Nat Struct Biol 10:342-348; Patel et al. (2004) J Biol Chem 279:16727-16735). The DxL motif consists of a conserved six-residue sequence (Phe/Tyr/Trp)-Asp-X-Leu-(V/T)-(R/G). However ligand-binding specificity of BR3 and BCMA appears to be determined by interactions outside this common motif (Gordon et al. (2003) Biochemistry 42:5977-5983; Liu et al. (2003) Nature 423, 49-56; Patel et al. (2004)) J Biol Chem 279:16727-16735).

Since both CRDs of TACI contain the DxL motif and have been shown qualitatively to interact with BAFF (Kim et al. (2003), supra), it is unclear if TACI's dual specificity for APRIL and BAFF, unlike the more restricted specificities of BR3 and BCMA, is achieved by using a different individual CRD for optimal APRIL- or BAFF-binding or by using both CRDs together to bind ligand with further contacts provided by the extra domain, compared to that observed for BCMA or BR3. Additionally, it had been postulated that TACI might bind adjacent BAFF trimers with its two different CRDs (Kim et al. (2003), supra).

### SUMMARY OF THE INVENTION

The present invention relates novel polypeptides with cysteine rich domain (“CRD”) sequences having improved binding to BAFF or APRIL or BAFF and APRIL. According to one embodiment, the CRD in the polypeptide comprises at least residues Xb-Q-H-Xc (SEQ ID NO:72) immediately C-terminal to the fourth cysteine residue of the CRD, wherein Xa is any amino acid residue except C, Xb is G, T, or N and Xc is P, L or M. According to another embodiment, the CRD in the polypeptide comprises residues G-Xg-Xh-P (SEQ ID NO:73) immediately C-terminal to the fourth cysteine residue of the CRD, wherein Xa is any amino acid residue except C; wherein Xg is any amino acid residue except C, E or P; wherein Xh is any amino acid except C, A, D or P.

According to another embodiment, the novel polypeptide having improved binding to BAFF or APRIL or BAFF and APRIL comprises a CRD sequence that is Formula I C-X2-X3-X4-X5-X6-X7-X8-X9-D-X11-L-X13-X14-X15-C-X17-X18-C-X20-X21-X22-C-G-X25-X26-P-X28-X29-X30-C-X32-X33-X34-C (SEQ ID NO:1), wherein X2-X3, X6-X9, X11, X13-X15, X17-X18, X20-X22 and X32-X34 are any amino acid except C; wherein X4 is any amino acid except C or is absent; wherein X5 is any amino acid except C or is absent; wherein X25 is any amino acid residue except C, E or P; wherein X26 is any amino acid except C, A, D or P; wherein X28 is K, Q, A, R, N, H or S; wherein X29 is any amino acid except C; wherein X30 is any amino acid except C or is absent; and wherein Formula I is not SEQ ID NO:4 or SEQ ID NO:6 or SEQ ID NO:70. According to one embodiment of Formula I, X30 is absent.

According to another embodiment, the novel polypeptide having improved binding to BAFF or APRIL or BAFF and APRIL comprises a CRD sequence that is Formula II C-X2-X3-X4-X5-X6-X7-X8-X9-D-X11-L-X13-X14-X15-C-X17-X18-C-X20-X21-X22-C-X24-Q-H-X27-X28-X29-X30-C-X32-X33-X34-C (SEQ ID NO:2), wherein X2-X3, X6-X9, X11, X13-X15, X17-X18, X20-X22 and X32-X34 are any amino acid except C; wherein X4 is any amino acid except C or is absent; wherein X5 is any amino acid except C or is absent; wherein X24 is G, T, or N; wherein X27 is P, L or M; wherein X28 is K, Q, A, R, N, H or S; wherein X29 is any amino acid except C; wherein X30 is any amino acid except C or is absent; and wherein Formula II is not SEQ ID NO:4 or SEQ ID NO:6 or SEQ ID NO:70. According to one embodiment of Formula II, X30 is absent.

According to another embodiment, the novel polypeptide having improved binding to BAFF or APRIL or BAFF and APRIL comprises a CRD sequence that is Formula III C-X2-X3-X4-X5-X6-X7-D-X9-L-X11-X12-X13-C-X15-X16-C-X18-X19-X20-C-X22-Q-H-X25-X26-X27-X28-C-X30-X31-X32-C (SEQ ID NO: 3), wherein X2-X7, X9, X11-X13, X15-X16, X18-X20 and X30-X32 are any amino acid except C; wherein X22 is G, T, or N; wherein X25 is P, L or M; wherein X26 is K, Q, A, R, N, H or S; wherein X27 is any amino acid except C; wherein X28 is any amino acid except C or is absent; and wherein Formula III is not SEQ ID NO:8 or SEQ ID NO:9. According to one embodiment of Formula III, X28 is absent.

According to another embodiment, the novel polypeptide having improved binding to BAFF or APRIL or BAFF and APRIL comprises an altered CRD1 sequence of a TACI polypeptide, wherein the altered CRD1 sequence comprises residues Xb-Q-H-Xc (SEQ ID NO:72) immediately C-terminal to the fourth cysteine residue, wherein Xa is any amino acid residue except C, Xb is G, T, or N and Xc is P, L or M, wherein the CRD sequence is not a CRD sequence of a naturally-occurring TACI polypeptide.

According to one embodiment, the CRD sequences of this invention comprise the following sequence between the between the fourth and fifth cysteine residues of the CRD: Xb-Q-H-Xc-Xd-Xe (SEQ ID NO:76) or Xb-Q-H-Xc-Xd-Xe-Xf (SEQ ID NO:77), wherein Xb is G, T, or N; wherein Xc is P, L or M; wherein Xd is K, Q, A, R, N, H or S; wherein Xe is any amino acid except C; and wherein Xf is any amino acid except C or is absent.

According to another embodiment, the present invention provides polypeptides that have increased specificity for APRIL or increased specificity for BAFF compared to a naturally occurring TACI CRD sequence. According to one embodiment, the polypeptides have increased specificity for APRIL or increased specificity for BAFF based on engineering the CRD sequences described above to have increased specificity, altering native TACI polypeptide CRD sequences (CRD1 or CRD2 sequence), or altering other BAFF-binding or APRIL-binding sequences having a DXL motif. According to one embodiment, the BAFF binding specificity is increased by altering at least the second residue N-terminal to the D-Xa-L motif of a CRD sequence. According to another embodiment, the BAFF binding specificity is increased by altering at least the first residue N-terminal to the D-Xa-L motif of a CRD sequence. According to another embodiment, the APRIL binding specificity is increased by altering the first residue N-terminal to the D-Xa-L motif of a CRD sequence. According to another embodiment, the APRIL binding specificity is increased by altering at least the second residue C-terminal to the D-Xa-L motif of a CRD sequence. According to another embodiment, the APRIL binding specificity is increased by altering the first residue N-terminal to the fourth cysteine of a CRD sequence. According to another embodiment, the APRIL or BAFF specificity is increased by altering a combination of those positions. According to one embodiment, the second residue N-terminal to the D-Xa-L motif is E or S. According to another embodiment, the first residue N-terminal to the D-Xa-L motif is V. According to another embodiment, the first residue N-terminal to the D-Xa-L motif is E. According to another embodiment, the first residue N-terminal to the fourth cysteine of the CRD is L. According to another embodiment, the second residue C-terminal to the D-Xa-L motif is selected from the group consisting of E, D, W, F and M.

The present invention provides TACI variant polypeptides. According to one embodiment, the TACI variant polypeptide comprises an amino acid sequence wherein residues 94-99 of human TACI replace residues 55-61 of human TACI (SEQ ID NO: 10).

According to one embodiment, a polypeptide of this invention binds BAFF with an IC50 value of 500 nM or less, 100 nM or less, 50 nM or less, 10 nM or less, 5 nM or less or 1 nM or less. According to another embodiment, a polypeptide of this invention binds APRIL with an IC50 value of 500 nM or less, 100 nM or less, 50 nM or less, 10 nM or less, 5 nM or less or 1 nM or less.

According to one embodiment, the polypeptide of this invention does not comprise a transmembrane domain or a cytoplasmic domain of a native TACI polypeptide. According to another embodiment, the polypeptide of this invention does not comprise a CRD1 of a native sequence human TACI polypeptide. According to another embodiment, the polypeptide of this invention does not comprise residues at least residues 157-end of a native sequence human TACI polypeptide. According to another embodiment, the polypeptide of this invention further comprises a sequence heterologous to a native TACI polypeptide sequence. According to another embodiment, the heterologous sequence is an Fc region of an IgG. According to another embodiment, the heterologous sequence is a leucine zipper. According to another embodiment, the polypeptide of this invention is an immunoadhesin.

According to another embodiment, the polypeptide of this invention is conjugated to an agent selected from the group consisting of a growth inhibitory agent, a cytotoxic agent, a detection agent, an agent that improves the bioavailability of the polypeptide and an agent that improves the half-life of the polypeptide. According to another embodiment, the polypeptide of this invention is conjugated to a non-proteinaceous polymer. According to another embodiment, the non-proteinaceous polymer comprises a polyethylene glycol polymer. According to another embodiment, the polypeptide of this invention the cytotoxic agent is selected from the group consisting of a toxin, an antibiotic and a radioactive isotope.

The present invention also provides nucleic acid molecules encoding the polypeptides, vectors comprising the nucleic acid molecules, host cells comprising the nucleic acid molecules or vectors comprising the nucleic acid molecules. According to one specific embodiment, the invention provides a method for producing a polypeptide comprising the step of culturing a host cell comprising the nucleic acid molecule according to this invention or a vector comprising the nucleic acid molecule, under conditions suitable for expressing the polypeptide from the vector. In a further embodiment, the polypeptide expressed by the host cell can be recovered.

The present invention comprises a composition comprising a polypeptide of this invention, optionally further comprising a pharmaceutically acceptable carrier. The composition can optionally further comprising at least a second therapeutic agent selected from the group consisting of an agent for treating an immune-related disease, a chemotherapeutic agent and a cytotoxic agent. In one embodiment, the composition further comprises an anti-CD20 antibody.

The present invention provides methods of using the nucleic acids and polypeptides in in vitro assays to screen for inhibitors of APRIL-TACI, APRIL-BCMA, BAFF-TACI, BAFF-BCMA and BAFF-BR3 interactions or signaling pathways, methods for inhibiting native APRIL binding to native TACI or BAFF binding to TACI in vitro or in a mammal, methods for treating an immune-related disease, methods for treating B cell malignancies, methods for treating B-cell regulated autoimmune disorders, methods for treating cancer and methods for treating a T-cell mediated disease in a mammal and methods for depleting B cells.

The present invention provides methods for inhibiting TACI biological activity such as TACI signaling pathway comprising the step of administering a polypeptide of this invention in an amount sufficient to inhibit TACI biological activity. The present invention provides methods for inhibiting BAFF biological activity comprising the step of administering a polypeptide of this invention in an amount sufficient to inhibit BAFF biological activity. The present invention provides methods for inhibiting the BR3 biological activity comprising the step of administering a polypeptide of this invention in an amount sufficient to inhibit BR3 biological activity. The present invention provides methods for inhibiting the BCMA biological activity comprising the step of administering a polypeptide of this invention in an amount sufficient to inhibit BCMA biological activity. The present invention provides methods for inhibiting the APRIL biological activity comprising the step of administering a polypeptide of this invention in an amount sufficient to inhibit APRIL biological activity.

The present invention provides methods for inhibiting BAFF-BR3 interactions comprising the step of administering a polypeptide of this invention in an amount sufficient to block or partially block the interaction between BAFF and BR3. The present invention provides methods for inhibiting BAFF-BCMA interactions comprising the step of administering a polypeptide of this invention in an amount sufficient to block or partially block the interaction between BAFF and BCMA. The present invention provides methods for inhibiting APRIL-BCMA interactions comprising the step of administering a polypeptide of this invention in an amount sufficient to block or partially block the interaction between APRIL and BCMA. The present invention provides methods for inhibiting BAFF-TACI interactions comprising the step of administering a polypeptide of this invention in an amount sufficient to block or partially block the interaction between BAFF and TACI. The present invention provides methods for inhibiting APRIL-TACI interactions comprising the step of administering a polypeptide of this invention in an amount sufficient to block or partially block the interaction between APRIL and TACI.

The methods of treatment or methods of depeleting B cells according to this invention can be carried out with the polypeptides of this invention alone or in combination with other therapies, such as anti-CD20 antibody therapy.

According to one specific embodiment, the invention provides a method for identifying an inhibitor of APRIL binding to TACI or BCMA or TACI and BCMA comprising the step of incubating a polypeptide according to this invention and an APRIL polypeptide in the presence of a candidate inhibitor and detecting the inhibitor that partially or fully blocks the binding of the polypeptide and APRIL. According to another specific embodiment, the invention provides a method for identifying an inhibitor of BAFF binding to TACI, BCMA or BR3 or any combination of those receptors comprising the step of incubating a polypeptide of this invention and a BAFF polypeptide in the presence of a candidate inhibitor and detecting the inhibitor that partially or fully blocks the binding of the polypeptide and BAFF.

According to another specific embodiment, the invention provides a method for inhibiting the TACI signaling pathway or biological activity comprising the step of administering a polypeptide of this invention in an amount sufficient to inhibit TACI signaling or biological activity. According to another specific embodiment, the invention provides a method for inhibiting the BCMA signaling pathway or biological activity comprising the step of administering a polypeptide of this invention in an amount sufficient to inhibit BCMA signaling or biological activity. According to another specific embodiment, the invention provides a method for inhibiting the BR3 signaling pathway or biological activity comprising the step of administering a polypeptide of this invention in an amount sufficient to inhibit BR3 signaling or biological activity. According to another specific embodiment, the invention provides a method for inhibiting the APRIL signaling pathway or biological activity comprising the step of administering a polypeptide of this invention in an amount sufficient to inhibit APRIL signaling or biological activity. According to another specific embodiment, the invention provides a method for inhibiting the BAFF signaling pathway or biological activity comprising the step of administering a polypeptide of this invention in an amount sufficient to inhibit BAFF signaling or biological activity. According to one embodiment, the inhibitor of the APRIL, BAFF, TACI, BCMA or BR3 signaling pathway or biological activity is inhibits binding of APRIL or BAFF or APRIL and BAFF to a receptor.

According to another specific embodiment, the invention provides a method for inhibiting native APRIL binding to native TACI comprising the step of providing an APRIL-binding polypeptide of this invention and contacting the native sequence APRIL polypeptide with the APRIL-binding polypeptide of this invention. According to another specific embodiment, the invention provides a method for inhibiting native BAFF binding to native TACI comprising the step of providing a BAFF-binding polypeptide of this invention and contacting the native sequence BAFF polypeptide with the BAFF-binding polypeptide of this invention. According to another specific embodiment, the invention provides a method for inhibiting native APRIL binding to native TACI in a mammal comprising the step of administering an APRIL-binding polypeptide according to this invention in an amount effective to inhibit binding between APRIL and TACI in the mammal. According to another specific embodiment, the invention provides a method for inhibiting native BAFF binding to native TACI in a mammal comprising the step of administering a BAFF-binding polypeptide according to this invention in an amount effective to inhibit binding between BAFF and TACI in the mammal.

According to another specific embodiment, the invention provides a method for treating an immune-related disease in a mammal suffering from an immune disease comprising the step of treating the mammal with a therapeutically effective amount of the polypeptide according to this invention. According to another specific embodiment, the immune related disease is selected from the group consisting of rheumatoid arthritis, multiple sclerosis, Sjogren's syndrome and systemic lupus erythematosis.

According to another specific embodiment, the invention provides a method for treating a cancer in a mammal suffering from a cancer comprising the step of treating the mammal with a therapeutically effective amount of the polypeptide according to this invention. According to another specific embodiment, the cancer is a B cell neoplasia. According to another specific embodiment, the cancer is selected from the group consisting of CLL, NHL, ALL or multiple myeloma.

According to another specific embodiment, the cancer is a gastrointestinal cancer or a glioblastoma.

According to another specific embodiment, the invention provides a a method for treating a T-cell mediated disease in a mammal suffering from a T-cell mediated disease comprising the step of treating the mammal with a therapeutically effective amount of the polypeptide of this invention. According to another specific embodiment, the T-cell mediated disease is selected from the group consisting of graft rejection, graft verses host disease (GVHD) and inflammation.

According to another specific embodiment, the invention provides a method for treating an immune-related disease comprising the step of administering a therapeutically effective amount of a polypeptide of this invention. According to another specific embodiment, the invention provides a method for treating B cell malignancies or cancer comprising the step of administering a therapeutically effective amount of a polypeptide of this invention. According to another specific embodiment, the invention provides a method for treating B-cell regulated autoimmune disorders comprising the step of administering a therapeutically effective amount of a polypeptide of this invention. According to another specific embodiment, the invention provides a method for depleting B cells comprising the step of administering an amount of a polypeptide of this invention sufficient to decrease B-cell levels. According to one embodiment, the B cell levels are decreased in the sera. Patients to be treated with the polypeptides of this invention can also be treated with one or more other therapeutic agents (e.g., anti-CD20 antibodies, chemotherapeutic agents). The treatment methods of the invention comprise a combination of concurrently and/or sequentially administering the anti-CD20 antibody or anti-CD20 antagonist and a polypeptide of this invention.

The present invention also provides kits and articles of manufacture comprising the polypeptides of this invention.

### BRIEF DESCRIPTION OF THE FIGURES

FIG. 1 shows a schematic of the domain structure of full-length TACI, its alternative splice variant shortTACI, and recombinant proteins, TACI_d1d2, TACI_d1 and TACI_d2.

FIG. 2 shows that shortTACI can induce NF-KB activation through either APRIL or BAFF.

FIG. 3 shows APRIL- and BAFF-binding by TACI variants. Competitive surface plasmon resonance experiments to measure binding to APRIL or BAFF were performed as described in the Example section. (A) Competitive inhibition of APRIL-binding to BCMA-Fc by TACI variants: TACI_d1 (filled circle), TACI_d2 (filled square), TACI_d1d2 (open triangle). (B) IC₅₀ values for competitive binding to APRIL and BAFF are shown as the mean of two (TACI_d1) or three (TACI_d1d2, TACI_d2) independent experiments. *indicates that an interaction was observed between TACI_d1 and BAFF, but the binding curve could not be fitted adequately to derive an accurate IC₅₀ value.

FIG. 4 shows a trimer of mouse APRIL (residues 105-241) bound to three copies of human TACI_d2. In this orientation, the membrane of the TACI-presenting cell would be at the bottom of the figure.

FIG. 5 shows an open book view of the APRIL-TACI_d2 interface. APRIL and one copy of TACI_d2 are rendered as molecular surfaces. Residues in the interface are darker colored depending upon percent of accessible surface area buried upon complex formation.

FIG. 6 shows sequence alignment of TACI, BR3 and BCMA CRDs. Secondary structural elements of TACI_d2 and BCMA when bound to APRIL are indicated above and below their respective sequences. Regions near cysteine residues positions are highlighted in dashed line bars and their general connectivity in TACI and BCMA is shown above the alignment. The cysteine connectivity in TACI_d1 is expected to be the same as in TACI_d2. TACI_d2, BCMA, and BR3 residues. Receptor residues that have F values >6 in shotgun alanine scanning are bolded. TACI_d2, BCMA and BR3 residues which bury >50% accessible surface area on binding APRIL (TACI_d2, BCMA), or BAFF (BR3) are in solid line bars. BCMA residues that bury >50% accessible surface area in binding BAFF are underlined. Every fifth TACI_d2 residue is marked by a dot above the alignment.

FIG. 7 shows the results of a shotgun alanine-scanning mutagenesis of TACI_d2 binding to BAFF or APRIL. The normalized frequency ratios (F) observed for each of the scanned positions in TACI_d2 obtained from sequences of positive clones after two rounds of selection for binding to APRIL (white bars) or BAFF (textured bars) are plotted. Those bars with an asterisk (*) above indicate values that represent a lower limit since Ala was not observed at these positions.

FIG. 8 shows APRIL- and BAFF-binding by TACI disulfide linked variants (A) a competitive inhibition assay and (B) IC50 values. The asterick indicates that an interaction was observed between TACI_d1 and BAFF, but the binding curve could not be fitted adequately to derive an accurate IC50 value.

### DETAILED DESCRIPTION OF THE INVENTION

The present invention relates, inter alia, to novel polypeptides and TACI variants arising from insight from structural and functional studies into the CRD1 and CRD2 domains of a TACI polypeptide. The present invention includes novel polypeptide sequences, methods for generating altered CRD1 sequences with improved ability to bind APRIL, BAFF or APRIL and BAFF and methods for generating altered CRD sequences with increased specificity for binding APRIL or BAFF as compared to wild-type TACI sequences.

According to one embodiment, in the polypeptide comprising the sequence of Formula I C-X2-X3-X4-X5-X6-X7-X8-X9-D-X11-L-X13-X14-X15-C-X17-X18-C-X20-X21-X22-C-G-X25-X26-P-X28-X29-X30-C-X32-X33-X34-C (SEQ ID NO:1), X8 is not F or Y. According to another embodiment, in the Formula I sequence, X9 is not F, W or Y. According to another embodiment, in the Formula I sequence, X14 is not G, H or R. According to another embodiment, in the Formula I sequence, X22 is not I, R or T. According to another embodiment, in the Formula I sequence, X2 is selected from the group consisting of R, A, G and P. According to another embodiment, in the Formula I sequence, X3 is selected from the group consisting of K, A, E, T. According to another embodiment, in the Formula I sequence, X4 is selected from the group consisting of A, E and absent. According to another embodiment, in the Formula I sequence, X5 is selected from the group consisting of Q, A, E, P and absent. According to another embodiment, in the Formula I sequence, X6 is G or A. According to another embodiment, in the Formula I sequence, X7 is selected from the group consisting of K, A, E or T. According to another embodiment, in the Formula I sequence, X8 is selected from the group consisting of F, A, V, I, M, E, S, T and Y. According to another embodiment, in the Formula I sequence, X9 is selected from the group consisting of Y, A, F, W, L, I, P, V and E. According to another embodiment, in the Formula I sequence, X13 is L or V. According to another embodiment, in the Formula I sequence, X14 is selected from the group consisting of R, L, A, K, F, H, M, N, T, Y, G, V, D, E and W. According to another embodiment, in the Formula I sequence, X15 is D or A. According to another embodiment, in the Formula I sequence, X17 is I or V. According to another embodiment, in the Formula I sequence, X18 is S or A. According to another embodiment, in the Formula I sequence, X2 is A. According to another embodiment, in the Formula I sequence, X21 is S or A. According to another embodiment, in the Formula I sequence, X22 is selected from the group consisting of I, V, T, A and L. According to another embodiment, in the Formula I sequence, X32 is A. According to another embodiment, in the Formula I sequence, X33 is selected from the group consisting of Y, A, D and S. According to another embodiment, in the Formula I sequence, X34 is selected from the group consisting of F, A, S and V.

According to one embodiment, in the polypeptide comprising the sequence of Formula II C-X2-X3-X4-X5-X6-X7-X8-X9-D-X11-L-X13-X14-X15-C-X17-X18-C-X20-X21-X22-C-X24-Q-H-X27-X28-X29-X30-C-X32-X33-X34-C (SEQ ID NO:2), X8 is not F or Y. According to another embodiment, in the Formula II sequence, X9 is not F, W or Y. According to another embodiment, in the Formula II sequence, X14 is not G, H or R. According to another embodiment, in the Formula II sequence, X22 is not I, R or T. According to another embodiment, in the Formula II sequence, X2 is selected from the group consisting of R, A, G and P. According to another embodiment, in the Formula II sequence, X3 is selected from the group consisting of K, A, E, T. According to another embodiment, in the Formula II sequence, X4 is selected from the group consisting of A, E and absent. According to another embodiment, in the Formula II sequence, X5 is selected from the group consisting of Q, A, E, P and absent. According to another embodiment, in the Formula II sequence, X6 is G or A. According to another embodiment, in the Formula II sequence, X7 is selected from the group consisting of K, A, E or T. According to another embodiment, in the Formula II sequence, X8 is selected from the group consisting of F, A, V, I, M, E, S, T and Y. According to another embodiment, in the Formula II sequence, X9 is selected from the group consisting of Y, A, F, W, L, I, P, V and E. According to another embodiment, in the Formula II sequence, X13 is L or V. According to another embodiment, in the Formula II sequence, X14 is selected from the group consisting of R, L, A, K, F, H, M, N, T, Y, G, V, D, E and W. According to another embodiment, in the Formula II sequence, X15 is D or A. According to another embodiment, in the Formula II sequence, X17 is I or V. According to another embodiment, in the Formula II sequence, X18 is S or A. According to another embodiment, in the Formula II sequence, X2 is A. According to another embodiment, in the Formula II sequence, X21 is S or A. According to another embodiment, in the Formula II sequence, X22 is selected from the group consisting of I, V, T, A and L. According to another embodiment, in the Formula II sequence, X32 is A. According to another embodiment, in the Formula II sequence, X33 is selected from the group consisting of Y, A, D and S. According to another embodiment, in the Formula II sequence, X34 is selected from the group consisting of F, A, S and V.

According to one embodiment, in the polypeptide comprising the sequence of Formula III: C-X2-X3-X4-X5-X6-X7-D-X9-L-X11-X12-X13-C-X15-X16-C-X18-X19-X20-C-X22-Q-H—X25-X26-X27-X28-C-X30-X31-X32-C (SEQ ID NO: 3), X6 is not F or Y. According to another embodiment, in the Formula III sequence, X7 is not F, W or Y. According to another embodiment, in the Formula III sequence, X12 is not G, H or R. According to another embodiment, in the Formula III sequence, X20 is not I, R or T. According to another embodiment, in the Formula III sequence, X2 is selected from the group consisting of R, A, G and P. According to another embodiment, in the Formula III sequence, X3 is selected from the group consisting of K, A, E, T. According to another embodiment, in the Formula III sequence, X4 is selected from the group consisting of G, A, E and absent. According to another embodiment, in the Formula III sequence, X5 is selected from the group consisting of K, Q, A, E, P, T and absent. According to another embodiment, in the Formula III sequence, X6 is selected from the group consisting of F, A, V, I, M, E, S, T and Y. According to another embodiment, in the Formula III sequence, X7 is selected from the group consisting of Y, A, F, W, L, I, P, V and E. According to another embodiment, in the Formula III sequence, X11 is L or V. According to another embodiment, in the Formula III sequence, X12 is selected from the group consisting of R, L, A, K, F, H, M, N, T, Y, G, V, D, E and W. According to another embodiment, in the Formula III sequence, X13 is D or A. According to another embodiment, in the Formula III sequence, X15 is I or V. According to another embodiment, in the Formula III sequence, X16 is S or A. According to another embodiment, in the Formula III sequence, X18 is A. According to another embodiment, in the Formula III sequence, X19 is S or A. According to another embodiment, in the Formula III sequence, X20 is selected from the group consisting of I, V, T, A and L. According to another embodiment, in the Formula III sequence, X30 is A. According to another embodiment, in the Formula III sequence, X31 is selected from the group consisting of Y, A, D and S. According to another embodiment, in the Formula III sequence, X32 is selected from the group consisting of F, A, S and V.

Polypeptides included in this invention are those comprising at least one of any one of the following sequences: CRKEQGKEYDHLLRDCISCASICGQHPKQCAYFC (SEQ ID NO:15), CRKEQGKSYDHLLRDCISCASICGQHPKQCAYFC (SEQ ID NO: 16), CRKEQGKFVDHLLRDCISCASICGQHPKQCAYFC (SEQ ID NO: 17), CRKEQGKEVDHLLRDCISCASICGQHPKQCAYFC (SEQ ID NO: 18), CRKEQGKSVDHLLRDCISCASICGQHPKQCAYFC (SEQ ID NO: 19), CPEEQYWDPLLGTCMSCKTICGQHPKQCAAFC (SEQ ID NO:20), CPEEQEWDPLLGTCMSCKTICGQHPKQCAAFC (SEQ ID NO:21), CPEEQSWDPLLGTCMSCKTICGQHPKQCAAFC (SEQ ID NO:22), CPEEQYVDPLLGTCMSCKTICGQHPKQCAAFC (SEQ ID NO:23), CPEEQEVDPLLGTCMSCKTICGQHPKQCAAFC (SEQ ID NO:24), CPEEQSVDPLLGTCMSCKTICGQHPKQCAAFC (SEQ ID NO:25), CRKEQGKFEDHLLRDCISCASICGQHPKQCAYFC (SEQ ID NO:26), CRKEQGKFYDHLLEDClSCASICGQHPKQCAYFC (SEQ ID NO:27), CRKEQGKFYDHLLWDCISCASICGQHPKQCAYFC (SEQ ID NO:28), CRKEQGKFYDHLLDDCISCASICGQHPKQCAYFC (SEQ ID NO:29), CRKEQGKFYDHLLFDCISCASICGQHPKQCAYFC (SEQ ID NO:30), CRKEQGKFYDHLLMDCISCASICGQHPKQCAYFC (SEQ ID NO:31), CRKEQGKFYDHLLRDCISCASLCGQHPKQCAYFC (SEQ ID NO:32), CRKEQGKFEDHLLEDClSCASICGQHPKQCAYFC (SEQ ID NO:33), CRKEQGKFEDHLLWDCISCASICGQHPKQCAYFC (SEQ ID NO:34), CRKEQGKFEDHLLDDCISCASICGQHPKQCAYFC (SEQ ID NO:35), CRKEQGKFEDHLLFDCISCASICGQHPKQCAYFC (SEQ ID NO:36), CRKEQGKFEDHLLMDCISCASICGQHPKQCAYFC (SEQ ID NO:37), CRKEQGKFYDHLLEDClSCASLCGQHPKQCAYFC (SEQ ID NO:38), CRKEQGKFYDHLLWDCISCASLCGQHPKQCAYFC (SEQ ID NO:39), CRKEQGKFYDHLLDDCISCASLCGQHPKQCAYFC (SEQ ID NO:40), CRKEQGKFYDHLLFDCISCASLCGQHPKQCAYFC (SEQ ID NO:41), CRKEQGKFYDHLLMDCISCASLCGQHPKQCAYFC (SEQ ID NO:42), CRKEQGKFEDHLLEDCISCASLCGQHPKQCAYFC (SEQ ID NO:43), CRKEQGKFEDHLLWDCISCASLCGQHPKQCAYFC (SEQ ID NO:44), CRKEQGKFEDHLLDDCISCASLCGQHPKQCAYFC (SEQ ID NO:45), CRKEQGKFEDHLLFDCISCASLCGQHPKQCAYFC (SEQ ID NO:46), CRKEQGKFEDHLLMDCISCASLCGQHPKQCAYFC (SEQ ID NO:47), CPEEQYEDPLLGTCMSCKTICGQHPKQCAAFC (SEQ ID NO:48), CPEEQYWDPLLETCMSCKTICGQHPKQCAAFC (SEQ ID NO:49), CPEEQYWDPLLWTCMSCKTICGQHPKQCAAFC (SEQ ID NO:50), CPEEQYWDPLLDTCMSCKTICGQHPKQCAAFC (SEQ ID NO:51), CPEEQYWDPLLFTCMSCKTICGQHPKQCAAFC (SEQ ID NO:52), CPEEQYWDPLLMTCMSCKTICGQHPKQCAAFC (SEQ ID NO:53), CPEEQYWDPLLGTCMSCKTLCGQHPKQCAAFC (SEQ ID NO:54), CPEEQYEDPLLETCMSCKTICGQHPKQCAAFC (SEQ ID NO:55), CPEEQYEDPLLWTCMSCKTICGQHPKQCAAFC (SEQ ID NO:56), CPEEQYEDPLLDTCMSCKTICGQHPKQCAAFC (SEQ ID NO:57), CPEEQYEDPLLFTCMSCKTICGQHPKQCAAFC (SEQ ID NO:58), CPEEQYEDPLLMTCMSCKTICGQHPKQCAAFC (SEQ ID NO:59), CPEEQYWDPLLETCMSCKTLCGQHPKQCAAFC (SEQ ID NO:60), CPEEQYWDPLLWTCMSCKTLCGQHPKQCAAFC (SEQ ID NO:61), CPEEQYWDPLLDTCMSCKTLCGQHPKQCAAFC (SEQ ID NO:62), CPEEQYWDPLLFTCMSCKTLCGQHPKQCAAFC (SEQ ID NO:63), CPEEQYWDPLLMTCMSCKTLCGQHPKQCAAFC (SEQ ID NO:64), CPEEQYEDPLLETCMSCKTLCGQHPKQCAAFC (SEQ ID NO: 65), CPEEQYEDPLLWTCMSCKTLCGQHPKQCAAFC (SEQ ID NO:66), CPEEQYEDPLLDTCMSCKTLCGQHPKQCAAFC (SEQ ID NO:67), CPEEQYEDPLLFTCMSCKTLCGQHPKQCAAFC (SEQ ID NO:68) and CPEEQYEDPLLMTCMSCKTLCGQHPKQCAAFC (SEQ ID NO:69).

The terms “TACI” or “TACI polypeptide” or “TACI receptor” when used herein encompass “native sequence TACI polypeptides” and “TACI variants” (which are further defined herein). “TACI” is a designation given to those polypeptides comprising the amino acid sequences of SEQ ID NO:10, SEQ ID NO:11 and SEQ ID NO:12, and homologs, variants and fragments thereof, nucleic acid molecules comprising the amino acid sequences and variants thereof as well as fragments of the above. The TACI polypeptides of the invention can be isolated from a variety of sources, such as from human tissue types or from another source, or prepared by recombinant and/or synthetic methods.

A “native sequence” TACI polypeptide comprises a polypeptide having the same amino acid sequence as the corresponding TACI polypeptide derived from nature. Such native sequence TACI polypeptides can be isolated from nature or can be produced by recombinant and/or synthetic means. The term “native sequence TACI polypeptide” specifically encompasses naturally-occurring truncated, soluble or secreted forms (e.g., an extracellular domain sequence), naturally-occurring variant forms (e.g., alternatively spliced forms) and naturally-occurring allelic variants of the polypeptide. The TACI polypeptides of the invention include but are not limited to the polypeptides described in von Bulow et al., supra and WO98/39361 published Sep. 11, 1998, the spliced variant (referred to as “hTACI(265)” above and shown SEQ ID NO:12.

A TACI “extracellular domain” or “ECD” refers to a form of the TACI polypeptide which is essentially free of the transmembrane and cytoplasmic domains. Examples of ECD forms of TACI include those described in von Bulow et al., supra, WO 98/39361, WO 00/40716, WO 01/85782, WO 01/87979, WO 01/81417, amino acid residues 1-166 of SEQ ID NO: 10.

A “cysteine rich domain” or “CRD” as used herein refers to an amino acid sequence comprising six cysteine residues with a D-Xa-L sequence (“D-Xa-L” or “DXL” motif) between the first and second cysteine residues, wherein Xa or X refers to any amino acid except C.

A TACI “cysteine rich domain 1” or “CRD1” refers to the first cysteine rich domain of a mammalian TACI polypeptide having two CRD domains, e.g., residues 34-66 of human TACI (293aa) (SEQ ID NO:8) or residues 6-38 of mouse TACI (SEQ ID NO:9).

A TACI “cysteine rich domain 2” or “CRD2” refers to the second cysteine rich domain of a mammalian TACI polypeptide having two CRD domains, e.g., residues 71-104 of human TACI (293aa) or residues 25-58 of shortTACI, (SEQ ID NO:4), or residues 43-76 of mouse TACI (SEQ ID NO:6).

“TACI variant” means a polypeptide comprising a sequence that has at least about 70% amino acid sequence identity with the CRD2 amino acid sequence of a native sequence TACI and binds a native sequence BAFF polypeptide, native sequence APRIL polypeptide or both. Such TACI variant polypeptides include, for instance, TACI polypeptides wherein one or more amino acid residues are added, or deleted, at the N- and/or C-terminus, as well as within one or more internal domains, of the full-length amino acid sequence. Fragments of the TACI ECD that bind a native sequence BAFF polypeptide are also contemplated. Ordinarily, a TACI variant polypeptide will have a sequence that is at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with SEQ ID NO:4. The variant TACI sequences that have at least about 70% amino acid sequence identity with the amino acid sequence of SEQ ID NO:2 do not encompass native TACI polypeptide CRDs (e.g., SEQ ID NOs:4, 6, 8 and 9). Ordinarily, TACI variant polypeptides are at least about 32-34 amino acids in length.

The terms “BAFF,” “BAFF polypeptide,” “TALL-1” or “TALL-1 polypeptide,” “BLyS” when used herein encompass “native sequence BAFF polypeptides” and “BAFF variants”. “BAFF” is a designation given to those polypeptides which are encoded by any one of the amino acid sequences SEQ ID NO: 13 (human BAFF sequence or SEQ ID NO:71 (mouse BAFF sequence) and homologs and fragments and variants thereof, which have the biological activity of the native sequence BAFF. A biological activity of BAFF can be selected from the group consisting of promoting B cell survival, promoting B cell maturation and binding to BR3, BCMA or TACI. Variants of BAFF will preferably have at least 80% or any successive integer up to 100% including, more preferably, at least 90%, and even more preferably, at least 95% amino acid sequence identity with a native sequence of a BAFF polypeptide. A “native sequence” BAFF polypeptide comprises a polypeptide having the same amino acid sequence as the corresponding BAFF polypeptide derived from nature. For example, BAFF, exists in a soluble form following cleavage from the cell surface by furin-type proteases. Such native sequence BAFF polypeptides can be isolated from nature or can be produced by recombinant and/or synthetic means. The term “native sequence BAFF polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “BAFF” includes those polypeptides described in Shu et al., J. Leukocyte Biol., 65:680 (1999); GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., Science, 285:260-263 (1999); Schneider et al., J. Exp. Med., 189:1747-1756 (1999); Mukhopadhyay et al., J. Biol. Chem., 274:15978-15981 (1999).

The terms “APRIL” or “APRIL polypeptide” when used herein encompass “native sequence APRIL polypeptides” and “APRIL variants”. “APRIL” is a designation given to polypeptides having the sequence shown in SEQ ID NO: 14 and homologs and variants thereof, nucleic acid molecules encoding the sequence, and variants thereof as well as fragments of the above which have the biological activity of the native sequence APRIL. Variants of APRIL will preferably have at least 80%, more preferably, at least 90%, and even more preferably, at least 95% amino acid sequence identity with the native sequence APRIL polypeptide shown in SEQ ID NO: 14. A “native sequence” APRIL polypeptide comprises a polypeptide having the same amino acid sequence as the corresponding APRIL polypeptide derived from nature. Such native sequence APRIL polypeptides can be isolated from nature or can be produced by recombinant and/or synthetic means. The term “native sequence APRIL polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “APRIL” includes those polypeptides described in Hahne et al., J. Exp. Med., 188:1185-1190 (1998); GenBank Accession No. AF046888; WO 99/00518 published Jan. 7, 1999; WO 99/12965 published Mar. 18, 1999; WO 99/33980 published Jul. 8, 1999; WO 97/33902 published Sep. 18, 1997; WO 99/11791 published Mar. 11, 1999; EP 911,633 published Mar. 28, 1999; and WO99/50416 published Oct. 7, 1999.

Residues 8-41 of human BCMA are described in SEQ ID NO:70.

“Percent (%) amino acid sequence identity” with respect to the TACI polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code (Table 1) has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The amino acid sequences described herein are contiguous amino acid sequences unless otherwise specified.

Variations in polypeptides of this invention described herein, can be made, for example, using any of the techniques and guidelines for conservative and non-conservative mutations. Variations can be a substitution, deletion or insertion of one or more codons encoding the polypeptide that results in a change in the amino acid sequence of the polypeptide. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature native sequence.

The term “conservative” amino acid substitution as used within this invention is meant to refer to amino acid substitutions which substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. In general, substitutions within a group can be considered conservative with respect to structure and function. However, the skilled artisan will recognize that the role of a particular residue is determined by its context within the three-dimensional structure of the molecule in which it occurs. For example, Cys residues may occur in the oxidized (disulfide) form, which is less polar than the reduced (thiol) form. The long aliphatic portion of the Arg side chain can constitute a critical feature of its structural or functional role, and this may be best conserved by substitution of a nonpolar, rather than another basic residue. Also, it will be recognized that side chains containing aromatic groups (Trp, Tyr, and Phe) can participate in ionic-aromatic or “cation-pi” interactions. In these cases, substitution of one of these side chains with a member of the acidic or uncharged polar group may be conservative with respect to structure and function. Residues such as Pro, Gly, and Cys (disulfide form) can have direct effects on the main chain conformation, and often may not be substituted without structural distortions.

Conservative substitutions include the following specific substitutions based on the similarities in side chains and exemplary substitutions and preferred substitutions listed below. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gin (O)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: H is, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Non-conservative substitutions will entail exchanging a member of one of these classes for another class. Such substituted residues also may be introduced into the conservative substitution sites or, more preferably, into the remaining (non-conserved) sites.

The term “amino acid” within the scope of the present invention is used in its broadest sense and is meant to include the naturally occurring L alpha-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio (The Peptides: Analysis, Synthesis, Biology,) Eds. Gross and Meiehofer, Vol. 5 p 341, Academic Press, Inc, N.Y. 1983, which is incorporated herein by reference.

Peptides synthesized by the standard solid phase synthesis techniques described here, for example, are not limited to amino acids encoded by genes for substitutions involving the amino acids. Commonly encountered amino acids which are not encoded by the genetic code, include, for example, those described in International Publication No. WO 90/01940, as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Abe) for Met, Leu and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and H is; N-ethylglycine (EtGly) for Gly, Pro, and Ala; N-ethylglycine (EtGly) for Gly, Pro, and Ala; N-ethylasparigine (EtAsn) for Asn, and Gln; Hydroxyllysine (Hyl) for Lys; allohydroxyllysine (AHyl) for Lys; 3-(and 4)hydroxyproline (3Hyp, 4Hyp) for Pro, Ser, and Thr; allo-isoleucine (Ale) for Ile, Leu, and Val; -amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn or Or) for Lys, Arg and H is; Citrulline (Cit) and methionine sulfoxide (MS O) for Thr, Asn and Gln; -methylphenylalanine (MePhe), trimethylphenylalanine, halo (F, Cl, Br, and I)phenylalanine, triflourylphenylalanine, for Phe.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA. Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol, 150:1 (1976)]. If alanine substitution does not yield adequate amounts of variant, an isoteric amino acid can be used.

The term “increase specificity” as used herein refers to the increased preference of a polypeptide of this invention for binding one protein (APRIL, BAFF) over another, different protein (APRIL, BAFF) as compared to the naturally occurring TACI polypeptide sequence that binds to the same two proteins. Increased specificity can be achieved by, e.g., increasing the affinity of the polypeptide of this invention for the preferred protein, decreasing the affinity of the polypeptide of this invention for the non-preferred protein or a combination of increasing the affinity of the polypeptide of this invention for the preferred protein while decreasing the affinity of the polypeptide of this invention for the non-preferred protein.

The term “detecting” is intended to include determining the presence or absence of a substance or quantifying the amount of a substance. The term thus refers to the use of the materials, compositions, and methods of the present invention for qualitative and quantitative determinations. In general, the particular technique used for detection is not critical for practice of the invention.

For example, “detecting” according to the invention may include detecting: the presence or absence of a TACI, BCMA, BR3, BAFF or APRIL gene, mRNA molecules, or a TACI, BCMA, BR3, BAFF or APRIL polypeptide; a change in the levels of a TACI, BCMA, BR3, BAFF or APRIL polypeptide or amount bound to a target; a change in biological function/activity of a TACI, BCMA, BR3, BAFF or APRIL polypeptide e.g., ligand or receptor binding activity, intracellular signaling (such as NF-KB activation), tumor cell proliferation, B cell proliferation, or survival, etc.), e.g., using methods that are known in the art. In some embodiments, “detecting” may include detecting wild type TACI, BCMA, BR3, BAFF or APRIL levels (e.g., mRNA or polypeptide levels). Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, or more e.g., 100-fold.

As used herein, a subject to be treated is a mammal (e.g., human, non-human primate, rat, mouse, cow, horse, pig, sheep, goat, dog, cat, etc.). The subject may be a clinical patient, a clinical trial volunteer, an experimental animal, etc. The subject may be suspected of having or at risk for having a cancer or immune disease, be diagnosed with a cancer or immune disease, or be a control subject that is confirmed to not have a cancer. Many diagnostic methods for cancer and immune disease and the clinical delineation of cancer or immune diagnoses are known in the art. According to one preferred embodiment, the subject to be treated according to this invention is a human.

“Treating” or “treatment” or “alleviation” refers to measures, wherein the object is to prevent or slow down (lessen) the targeted pathologic condition or disorder or relieve some of the symptoms of the disorder. Those in need of treatment include can include those already with the disorder as well as those prone to have the disorder or those in whom the disorder is to be prevented. A subject or mammal is successfully “treated” for a cancer if, after receiving a therapeutic amount of a polypeptide according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the polypeptides of this invention can prevent growth and/or kill existing cancer cells, it can be cytostatic and/or cytotoxic. Reduction of these signs or symptoms may also be felt by the patient.

The term “therapeutically effective amount” refers to an amount of a polypeptide of this invention effective to “alleviate” or “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition of “treated” below. To the extent the drug may prevent growth and/or kill existing cancer cells, it may be cytostatic and/or cytotoxic.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM. For example, useful immunoadhesins according to this invention are polypeptides that comprise the BAFF binding portions of a polypeptide of this invention without the transmembrane or cytoplasmic sequences of the TACI receptor. In one embodiment, a polypeptide of this invention is fused to a constant domain of an immunoglobulin sequence. For example, a sequence of Formula I, II or III can be fused to an Fc region of an IgG molecule.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis, juvenile-onset rheumatoid arthritis, osteoarthritis, psoriatic arthritis, and ankylosing spondylitis), psoriasis, dermatitis including atopic dermatitis, chronic idiopathic urticaria, including chronic autoimmune urticaria, polymyositis/dermatomyositis, toxic epidermal necrolysis, scleroderma (including systemic scleroderma), sclerosis such as progressive systemic sclerosis, inflammatory bowel disease (IBD) (for example, Crohn's disease, ulcerative colitis, autoimmune inflammatory bowel disease), pyoderma gangrenosum, erythema nodosum, primary sclerosing cholangitis, episcleritis), respiratory distress syndrome, including adult respiratory distress syndrome (ARDS), meningitis, IgE-mediated diseases such as anaphylaxis and allergic and atopic rhinitis, encephalitis such as Rasmussen's encephalitis, uveitis or autoimmune uveitis, colitis such as microscopic colitis and collagenous colitis, glomerulonephritis (GN) such as membranous GN (membranous nephropathy), idiopathic membranous GN, membranous proliferative GN (MPGN), including Type I and Type II, and rapidly progressive GN, allergic conditions, allergic reaction, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, subacute cutaneous lupus erythematosus, lupus (including nephritis, cerebritis, pediatric, non-renal, discoid, alopecia), juvenile onset (Type I) diabetes mellitus, including pediatric insulin-dependent diabetes mellitus (IDDM), adult onset diabetes mellitus (Type II diabetes), multiple sclerosis (MS) such as spino-optical MS, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitis (including large vessel vasculitis (including polymyalgia rheumatica and giant cell (Takayasu's) arteritis), medium vessel vasculitis (including Kawasaki's disease and polyarteritis nodosa), CNS vasculitis, systemic necrotizing vasculitis, and ANCA-associated vasculitis, such as Churg-Strauss vasculitis or syndrome (CSS)), temporal arteritis, aplastic anemia, Coombs positive anemia, Diamond Blackfan anemia, hemolytic anemia or immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, antigen-antibody complex mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous, pemphigus (including vulgaris, foliaceus, and pemphigus mucus-membrane pemphigoid), autoimmune polyendocrinopathies, Reiter's disease, immune complex nephritis, chronic neuropathy such as IgM polyneuropathies or IgM-mediated neuropathy, thrombocytopenia (as developed by myocardial infarction patients, for example), including thrombotic thrombocytopenic purpura (TTP) and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, chronic thyroiditis (Hashimoto's thyroiditis), or subacute thyroiditis, autoimmune thyroid disease, idiopathic hypothyroidism, Addison's disease, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis, myasthenia gravis, cerebellar degeneration, limbic and/or brainstem encephalitis, neuromyotonia, opsoclonus or opsoclonus myoclonus syndrome (OMS), and sensory neuropathy, Sheehan's syndrome, autoimmune hepatitis, chronic hepatitis, lupoid hepatitis, chronic active hepatitis or autoimmune chronic active hepatitis, lymphoid interstitial pneumonitis, bronchiolitis obliterans (non-transplant) vs NSIP, Guillain-Barré syndrome, Berger's disease (IgA nephropathy), primary biliary cirrhosis, celiac sprue (gluten enteropathy), refractory sprue, dermatitis herpetiformis, cryoglobulinemia, amylotrophic lateral sclerosis (ALS; Lou Gehrig's disease), coronary artery disease, autoimmune inner ear disease (AIED)-, or autoimmune hearing loss, opsoclonus myoclonus syndrome (OMS), polychondritis such as refractory polychondritis, pulmonary alveolar proteinosis, amyloidosis, giant cell hepatitis, scleritis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, autoimmune hepatological disorder, fibromyalgia, multiple endocrine failure, Schmidt's syndrome, adrenalitis, gastric atrophy, presenile dementia, demyelinating diseases, Dressler's syndrome, alopecia arcata, CREST syndrome (calcinosis, Raynaud's phenomenon, esophageal dysmotility, sclerodactyl), and telangiectasia), male and female autoimmune infertility, ankylosing spondolytis, mixed connective tissue disease, Chagas' disease, rheumatic fever, recurrent abortion, farmer's lung, erythema multiforme, post-cardiotomy syndrome, Cushing's syndrome, bird-fancier's lung, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, endophthalmitis, erythema elevatum et diutinum, erytluoblastosis fetalis, eosinophilic faciitis, Shulman's syndrome, Felty's syndrome, flariasis, cyclitis such as chronic cyclitis, heterochronic cyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, and giant cell polymyalgia.

The term “cytotoxic agent” as used herein refers to a substance that inhibits or prevents the function of cells and/or causes destruction of cells. The term is intended to include radioactive isotopes (e.g. At211, I131, I125, Y90, Re 186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON. toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce GI arrest and M-phase arrest. Classical M-phase blockers include the vincas (vincristine and vinblastine), TAXOL® paclitaxel, and topo II inhibitors such as doxorubicin, epirubicin, daunorubicin, etoposide, and bleomycin. Those agents that arrest GI also spill over into S-phase arrest, for example, DNA alkylating agents such as tanoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antieioplastic drugs” by Murakaini et al. (W B Saunders: Philadelphia, 1995), especially p. 13.

A “conjugate” refers to any hybrid molecule, including fusion proteins and as well as molecules that contain both amino acid or protein portions and non-protein portions. Conjugates may be synthesized or engineered by a variety of techniques known in the art including, for example, recombinant DNA techniques, solid phase synthesis, solution phase synthesis, organic chemical synthetic techniques or a combination of these techniques. The choice of synthesis will depend upon the particular molecule to be generated. For example, a hybrid molecule not entirely “protein” in nature may be synthesized by a combination of recombinant techniques and solution phase techniques.

1. Polynucleotides, Vectors, Host Cells

According to some embodiments, the polypeptides of this invention are selected from the group consisting of: the peptides described herein, polypeptides incorporating one or more peptides as core regions, and covalently modified forms of the peptides and polypeptides (e.g., immunoadhesins, labeled polypeptides, protected polypeptides, conjugated polypeptides, fusion proteins, etc.). Many techniques that are employed for making these forms of polypeptides are known in the art and some are described herein. Many methods for labeling polypeptides and conjugating molecules to polypeptides are known in the art.

Compositions of the invention can be prepared using recombinant techniques known in the art. The description below relates to methods of producing such polypeptides by culturing host cells transformed or transfected with a vector containing the encoding nucleic acid and recovering the polypeptide from the cell culture. (See, e.g., Sambrook et al., Molecular Cloning: A Laboratory Manual (New York: Cold Spring Harbor Laboratory Press, 1989); Dieffenbach et al., PCR Primer: A Laboratory Manual (Cold Spring Harbor Laboratory Press, 1995)).

The nucleic acid (e.g., cDNA or genomic DNA) encoding the desired polypeptide may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in WO97/25428.

Expression and cloning vectors usually contain a promoter that is recognized by the host organism and is operably linked to the encoding nucleic acid sequence. Promoters are untranslated sequences located upstream (5′) to the start codon of a structural gene (generally within about 100 to 1000 bp) that control the transcription and translation of a particular nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to the encoding DNA by removing the promoter from the source DNA by restriction enzyme digestion and inserting the isolated promoter sequence into the vector.

Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, the ligation mixtures can be used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced using standard techniques known in the art. [See, e.g., Messing et al., Nucleic Acids Res., 9:309 (1981); Maxam et al., Methods in Enzymology, 65:499 (1980)].

Expression vectors that provide for the transient expression in mammalian cells of the encoding DNA may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector [Sambrook et al., supra]. Transient expression systems, comprising a suitable expression vector and a host cell, allow for the convenient positive identification of polypeptides encoded by cloned DNAs, as well as for the rapid screening of such polypeptides for desired biological or physiological properties.

Other methods, vectors, and host cells suitable for adaptation to the synthesis of the desired polypeptide in recombinant vertebrate cell culture are described in Gething et al., Nature, 293:620-625 (1981); Mantei et al., Nature, 281:40-46 (1979); EP 117,060; and EP 117,058.

Suitable host cells for cloning or expressing the DNA in the vectors herein include prokaryote, yeast, or higher eukaryote cells. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenifonnis (e.g., B. lichenifonnis 41P disclosed in DD 266,710 published 12 Apr. 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts for vectors. Suitable host cells for the expression of glycosylated polypeptide are derived from multicellular organisms. Examples of all such host cells are described further in WO97/25428.

Host cells are transfected and can be transformed with the above-described expression or cloning vectors and cultured in nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO4 and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Prokaryotic cells can be cultured in any suitable culture media, e.g., as described Sambrook et al., supra. Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

The expressed polypeptides may be recovered from the culture medium as a secreted polypeptide, although may also be recovered from host cell lysates when directly produced without a secretory signal. If the polypeptide is membrane-bound, it can be released from the membrane using a suitable detergent solution (e.g. Triton-X 100) or its extracellular region may be released by enzymatic cleavage.

When the polypeptide is produced in a recombinant cell other than one of human origin, it is free of proteins or polypeptides of human origin. However, it is usually necessary to recover or purify the polypeptide from recombinant cell proteins or polypeptides to obtain preparations that are substantially homogeneous. As a first step, the culture medium or lysate may be centrifuged to remove particulate cell debris. The following are procedures exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG.

### Phage Display

According to some embodiments, the polypeptides of this invention selected from the group consisting of: Formula I, Formula II or Formula III, may be utilized in phage display to derive other sequences with increased BAFF-binding, APRIL-binding or BAFF/APRIL binding capability and/or specificity.

Using the techniques of phage display allows the generation of large libraries of protein variants which can be rapidly sorted for those sequences that bind to a target molecule with high affinity. Nucleic acids encoding variant polypeptides are fused to a nucleic acid sequence encoding a viral coat protein, such as the gene III protein or the gene VIII protein. Monovalent phage display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phage display system, the gene fusion is expressed at low levels and wild type gene III proteins are also expressed so that infectivity of the particles is retained. Methods of generating peptide libraries and screening those libraries have been disclosed in many patents (e.g. U.S. Pat. No. 5,723,286, U.S. Pat. No. 5,432,018, U.S. Pat. No. 5,580,717, U.S. Pat. No. 5,427,908 and U.S. Pat. No. 5,498,530).

In some embodiments, Formula I, II, or III are expressed as peptide libraries on phage. The phage expressing the library of polypeptides of Formula I, II or III are then subjected to selection based on BAFF binding, APRIL binding or both. In some embodiments, the selection process involves allowing some phage to bind to biotinylated ligand (i.e., BAFF or APRIL) which is subsequently bound to a neutravidin plate. Phage bound to the plate through the ligand-biotin-neutravidin binding are recovered and propagated. In some embodiments, the phage are subject to several rounds of selection. In some embodiments, the phage is incubated with ligand-biotin, followed by the addition of unbiotinylated ligand as a competitive binder. Additional guidance of use of phage display in the context of the present invention is provided in the Examples.

### Polypeptides Fused or Conjugated to Heterologous Polypeptides

Immunoadhesin molecules comprising the polypeptides of this invention are further contemplated for use in the methods herein. In some embodiments, the molecule comprises a fusion of a polypeptide of this invention with an immunoglobulin or a particular region of an immunoglobulin. For a bivalent form of the immunoadhesin, such a fusion usefully comprises the Fc region of an IgG molecule. In a further embodiment, the Fc region is from a human IgG1 molecule. In some embodiments, the immunoglobulin fusion includes the hinge, CH₂ and CH₃, or the hinge, CH1, CH₂ and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

The simplest and most straightforward immunoadhesin design often combines the binding domain(s) of the adhesin (e.g. ligand binding polypeptide of this invention) with the Fc region of an immunoglobulin heavy chain. For example, a polypeptide comprising a sequence of Formula I, Formula II or Formula III, can be covalently linked to an Fc portion of an immunoglobulin. In addition, one or more of these polypeptides can be linked to one another and linked to an Fc portion of an immunoglobulin.

Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G1 (IgG1). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and CH2 and CH3 or (b) the CH1, hinge, CH2 and CH3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:

(a) ACL-ACL;

(b) ACH-(ACH, ACL-ACH, ACL-VHCH, or VLCL-ACH);

(c) ACL-ACH-(ACL-ACH, ACL-VHCH, VLCL-ACH, or VLCL-VHCH)

(d) ACL-VHCH-(ACH, or ACL-VHCH, or VLCL-ACH);

(e) VLCL-ACH-(ACL-VHCH, or VLCL-ACH); and

(f) (A-Y)n-(VLCL-VHCH)2,

wherein each A represents identical or different polypeptides comprising an amino acid sequence of Formula I, II or III, or combinations thereof;
VL is an immunoglobulin light chain variable domain;
VH is an immunoglobulin heavy chain variable domain;
CL is an immunoglobulin light chain constant domain;
CH is an immunoglobulin heavy chain constant domain;
n is an integer greater than 1;
Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the CH2 domain, or between the CH2 and CH3 domains. Similar constructs have been reported by Hoogenboom et al., Mol. Iimmunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the polypeptide of this invention.

The polypeptides of the present invention can also be modified in a way to form chimeric molecules by fusing the polypeptide to another, heterologous polypeptide or amino acid sequence. According to some embodiments, such heterologous polypeptide or amino acid sequence is one which acts to oligimerize the chimeric molecule. In some embodiments, such a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an “-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

### Construction of Peptide-Polymer Conjugates

In some embodiments the strategy for the conjugation of a polymer, (e.g, PEGylation) of synthetic peptides consists of combining, through forming a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

a. Peptide Reactive Sites

In some embodiments, a peptide is covalently bonded via one or more of the amino acid residues of the peptide to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the peptide. Potential reactive sites include: N-terminal amino group, epsilon amino groups on lysine residues, as well as other amino, imino, carboxyl, sulfhydryl, hydroxyl, and other hydrophilic groups. It will be understood, however, that the type and amount of the reactive group chosen, as well as the type of polymer employed, to obtain optimum results, will depend on the particular peptide employed to avoid having the reactive group react with too many particularly active groups on the peptide. In some embodiments, a reactive residue, (e.g., lysine (K), a modified, non-natural amino acid, or other small molecule) may be substituted at a position suitable for conjugation.

In some embodiments, the peptide comprises the sequence of Formula I, II or III have a terminal reactive group. In some embodiments, the peptide comprises at least one and can be more than one of a polypeptide comprising a sequence of Formula I, II or III. The polypeptides that are linked together can have the same sequence or have different sequences and a terminal reactive group. In some embodiments, these polypeptides can be joined to one another, optionally, through the use of a linker.

While conjugation may occur at any reactive amino acid on the polypeptide, in some embodiments, the reactive amino acid is lysine, which is linked to the reactive group of the activated polymer through its free epsilon-amino group, or glutamic or aspartic acid, which is linked to the polymer through an amide bond. In some embodiments, the reactive amino acids of the peptide are not cysteine residues at positions X₂ and X₁₂.

The degree of polymer conjugation with each peptide will vary depending upon the number of reactive sites on the peptide, the molecular weight, hydrophilicity and other characteristics of the polymer, and the particular peptide derivatization sites chosen. In some embodiments, the conjugate has a final molar ratio of 1 to 10 polymer molecules per peptide molecule, but greater numbers of polymer molecules attached to the peptides of the invention are also contemplated. In some embodiments, each conjugate contains one polymer molecule. The desired amount of derivatization is easily achieved by using an experimental matrix in which the time, temperature and other reaction conditions are varied to change the degree of substitution, after which the level of polymer substitution of the conjugates is determined by size exclusion chromatography or other means known in the art.

b. Activated Polymers

In some embodiments, the polymer contains only a single group which is reactive. This helps to avoid cross-linking of protein molecules. However, it is within the scope herein to maximize reaction conditions to reduce cross-linking, or to purify the reaction products through gel filtration or ion exchange chromatography to recover substantially homogenous derivatives. In other embodiments, the polymer contains two or more reactive groups for the purpose of linking multiple peptides to the polymer backbone. Again, gel filtration or ion exchange chromatography can be used to recover the desired derivative in substantially homogeneous form. In some embodiments, the polymer is covalently bonded directly to the peptide without the use of a multifunctional (ordinarily bifunctional) crosslinking agent. In some embodiments, there is a 1:1 molar ratio of PEG chain to peptide.

The covalent modification reaction may take place by any appropriate method generally used for reacting biologically active materials with inert polymers, preferably at about pH 5-9, more preferably 7-9 if the reactive groups on the peptide are lysine groups. Generally, the process involves preparing an activated polymer (the polymer typically having at least one terminal hydroxyl group to be activated), preparing an active substrate from this polymer, and thereafter reacting the peptide with the active substrate to produce the peptide suitable for formulation. The above modification reaction can be performed by several methods, which may involve one or more steps. Examples of modifying agents that can be used to produce the activated polymer in a one-step reaction include cyanuric acid chloride (2,4,6-trichloro-5-triazine) and cyanuric acid fluoride.

In some embodiments, the modification reaction takes place in two steps wherein the polymer is reacted first with an acid anhydride such as succinic or glutaric anhydride to form a carboxylic acid, and the carboxylic acid is then reacted with a compound capable of reacting with the carboxylic acid to form an activated polymer with a reactive ester group that is capable of reacting with the peptide. Examples of such compounds include N-hydroxysuccinimide, 4-hydroxy-3-nitrobenzene sulfonic acid, and the like, and preferably N-hydroxysuccinimide or 4-hydroxy-3-nitrobenzene sulfonic acid is used. For example, monomethyl substituted PEG may be reacted at elevated temperatures, preferably about 100-110° C. for four hours, with glutaric anhydride. The monomethyl PEG-glutaric acid thus produced is then reacted with N-hydroxysuccinimide in the presence of a carbodimide reagent such as dicyclohexyl or isopropyl carbodiimide to produce the activated polymer, methoxypolyethylene glycolyl-N-succinimidyl glutarate, which can then be reacted with the GH. This method is described in detail in Abuchowski et al., Cancer Biochem. Biophys., 7: 175-186 (1984). In another example, the monomethyl substituted PEG may be reacted with glutaric anhydride followed by reaction with 4-hydroxy-3-nitrobenzene sulfonic acid (HNSA) in the presence of dicyclohexyl carbodimide to produce the activated polymer. HNSA is described by Bhatnagar et al., Peptides: Synthesis-Structure-Function. Proceedings of the Seventh American Peptide Symposium, Rich et al. (eds.) (Pierce Chemical Co., Rockford Ill., 1981), p. 97-100, and in Nitecki et al., High-Technology Route to Virus Vaccines (American Society for Microbiology: 1986) entitled “Novel Agent for Coupling Synthetic Peptides to Carriers and Its Applications.”

In some embodiments, covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG.). Carboxyl groups are derivatized by coupling PEG-amine using carbodiimide. Sulfhydryl groups are derivatized by coupling to maleimido-substituted PEG (e.g. alkoxy-PEG amine plus sulfosuccinimidyl 4-(N-maleimidomethyl)cyclohexane-1-carboxylate) as described in WO 97/10847 published Mar. 27, 1997, or PEG-maleimide commercially available from Nektar Technologies, San Carlos, Calif. (formerly Shearwater Polymers, Inc.). Alternatively, free amino groups on the peptide (e.g. epsilon amino groups on lysine residues) may be coupled to N-hydroxysucciminidyl substituted PEG (PEG-NHS available from Nektar Technologies;) or can be thiolated with 2-imino-thiolane (Traut's reagent) and then coupled to maleimide-containing derivatives of PEG as described in Pedley et al., Br. J. Cancer, 70: 1126-1130 (1994).

Many inert polymers, including but not limited to PEG, are suitable for use in pharmaceuticals. See, e.g., Davis et al., Biomedical Polymers: Polymeric Materials and Pharmaceuticals for Biomedical Use, pp. 441-451 (1980). In some embodiments of the invention, a non-proteinaceous polymer is used. The nonproteinaceous polymer is typically a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers fall within the scope of this invention, e.g. polyvinylalcohol and polyvinylpyrrolidone. Particularly useful are polyalkylene ethers such as polyethylene glycol (PEG); polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

The polymer prior to conjugation need not be, but preferably is, water soluble, but the final conjugate is preferably water-soluble. Preferably, the conjugate exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion, injection, or inhalation if the conjugate is intended to be administered by such routes.

The molecular weight of the polymer can range up to about 100,000 D, and preferably is at least about 500 D, or at least about 1,000 D, or at least about 5,000 D. In some embodiments, the PEG or other polymer has a molecular weight in the range of 5000 to 20,000 D. The molecular weight chosen can depend upon the effective size of the conjugate to be achieved, the nature (e.g. structure, such as linear or branched) of the polymer, and the degree of derivatization, i.e. the number of polymer molecules per peptide, and the polymer attachment site or sites on the peptide. In some embodiments, branched PEG's may used to induce a large increase in effective size of the peptides. PEG or other polymer conjugates may be utilized to increase half-life, increase solubility, stabilize against proteolytic attack, and reduce immunogenicity.

Functionalized PEG polymers to modify the peptides of the invention are available from Nektar Technologies of San Carlos, Calif. (formerly Shearwater Polymers, Inc.). Such commercially available PEG derivatives include, but are not limited to, amino-PEG, PEG amino acid esters. PEG-N-hydroxysuccinamide chemistry (NHS), PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-xycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer.

c. Characterization of Conjugates.

The conjugates may be characterized by SDS-PAGE, gel filtration, NMR, tryptic mapping, liquid chromatography-mass spectrophotometry, and in vitro biological assays. For example, the extent of PEG conjugation may be shown by SDS-PAGE and gel filtration, and then analyzed by NMR, which has a specific resonance peak for the methylene hydrogens of PEG. The number of PEG groups on each molecule can be calculated from the NMR spectrum or mass spectrometry. Polyacrylamide gel electrophoresis in 10% SDS is appropriately run in 10 mM Tris-HCl pH 8.0, 100 mM NaCl as elution buffer. To demonstrate which residue is PEGylated, tryptic mapping can be performed. Thus, PEGylated peptides are digested with trypsin at the protein/enzyme ratio of 100 to 1 in mg basis at 37° C. for 4 hours in 100 mM sodium acetate, 10 mM Tris-HCl, 1 mM calcium chloride, pH 8.3, and acidified to pH<4 to stop digestion before separating on HPLC Nucleosil C-18 (4.6 mm.times.150 mm, 5.mu., 100A). The chromatogram is compared to that of non-PEGylated starting material. Each peak can then be analyzed by mass spectrometry to verify the size of the fragment in the peak. The fragment(s) that carried PEG groups are usually not retained on the HPLC column after injection and disappear from the chromatograph. Such disappearance from the chromatograph is an indication of PEGylation on that particular fragment that should contain at least one lysine residue. PEGylated peptides may then be assayed for ability to bind to the BAFF or APRIL by conventional methods.

In some embodiments, conjugates are purified by ion-exchange chromatography, (e.g, ion exchange HPLC. The chemistry of many of the electrophilically activated PEG's results in a reduction of amino group charge of the PEGylated product. Thus, high resolution ion exchange chromatography can be used to separate the free and conjugated proteins, and to resolve species with different levels of PEGylation. In fact, the resolution of different species (e.g. containing one or two PEG residues) is also possible due to the difference in the ionic properties of the unreacted amino acids. In one embodiment, species with difference levels of PEGylation are resolved according to the methods described in WO 96/34015 (International Application No. PCT/US96/05550 published Oct. 31, 1996). Heterologous species of the conjugates are purified from one another in the same fashion.

In some embodiments, PEG-N-hydroxysuccinamide (NHS) reacts with a primary amine (e.g. lysines and the N-terminus). In some embodiments, PEG-NHS reacts with a C-terminal lysine (K) of the polypeptide. In some embodiments, the lysine residue is added to the C-terminus of the 17-mer polypeptide, while in other embodiments, X₁₇ is substituted with lysine. In some embodiments, the polymer reacts with the N-terminus. In a preferred embodiment, the conjugate is generated by utilizing the derivatization and purification methods described in the Examples below.

In one aspect, the invention provides any of the above-described conjugates formed by its component parts, i.e. one or more peptide(s) covalently attached to one or more polymer molecule(s), without any extraneous matter in the covalent molecular structure of the conjugate.

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Assays

Peripheral B-cell concentrations are determined by a FACS method that count CD3−/CD40+ cells. The percent of CD3−CD40+B cells of total lymphocytes in samples can be obtained by the following gating strategy. The lymphocyte population is marked on the forward scatter/side scatter scattergram to define Region 1 (R1). Using events in R1, fluorescence intensity dot plots are displayed for CD40 and CD3 markers. Fluorescently labeled isotype controls are used to determine respective cutoff points for CD40 and CD3 positivity.

### FACS Analysis

Half million cells are washed and resuspended in 100 l of FACS buffer, which is phosphate buffered saline with 1% BSA, containing 5 l of staining or control antibody. All the staining antibodies, including isotype controls, are obtained from PharMingen, San Diego, Calif. Human CD₂O expression is assessed by staining with Rituxan® along with FITC-conjugated anti-human IgG1 secondary antibody. FACS analysis is conducted using FACScan and Cell Quest (Becton Dickinson Immunocytometry Systems, San Jose, Calif.). All the lymphocytes are defined in the forward and side light scatterings, while all the B lymphocytes are defined with the expression of B220 on the cell surface.

B cell depletion and recovery are assessed by analyzing peripheral B cell counts and analysis of hCD20+ B cells by FACS in the spleen, lymph node and bone marrow on a daily basis for the first week after injection and thereafter on a weekly basis. Serum levels of the injected polypeptide of this invention are monitored.

### Disease Treatment

### Diseases

The polypeptides of the invention are useful to treat B cell malignancies and B-cell regulated autoimmune disorders. B-cell regulated autoimmune diseases include arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliaceus), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, Lymphoid interstitial pneumonitis (HIV), bronchiolitis obliterans (non-transplant) vs NSIP, Guillain-Barre' Syndrome, Large Vessel Vasculitis (including Polymyalgia Rheumatica and Giant Cell (Takayasu's) Arteritis), Medium Vessel Vasculitis (including Kawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, coronary artery disease.

The B cell neoplasms include CD₂O-positive Hodgkin's disease including lymphocyte predominant Hodgkin's disease (LPHD); BR3-positive lymphomas and leukemias, TACI-positive lymphomas and leukemias, multiple myelomas, BCMA-positive lyphomas and leukemias, non-Hodgkin's lymphoma (NHL); follicular center cell (FCC) lymphomas; acute lymphocytic leukemia (ALL); chronic lymphocytic leukemia (CLL); Hairy cell leukemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, marginal zone lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment and is characterized by CD20-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

In specific embodiments, the TACI polypeptides, and optionally in combination with CD20 binding antibodies, are used to treat non-Hodgkin's lymphoma (NHL), lymphocyte predominant Hodgkin's disease (LPHD), chronic lymphocytic leukemia (CLL), small lymphocytic lymphoma (SLL) which is a type of non-Hodgkin's lymphoma (NHL), rheumatoid arthritis and juvenile rheumatoid arthritis, systemic lupus erythematosus (SLE) including lupus nephritis, Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

The desired level of B cell depletion will depend on the disease. For the treatment of a BAFF or BR3 positive cancer, it may be desirable to maximize the depletion of the B cells which are the target of the polypeptides of the invention. Thus, for the treatment of a BAFF or BR3-positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. Preferably, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

For treatment of an autoimmune disease, it may be desirable to modulate the extent of B cell depletion depending on the disease and/or the severity of the condition in the individual patient, by adjusting the dosage of the polypeptide of this invention. Thus, B cell depletion can but does not have to be complete. Or, total B cell depletion may be desired in initial treatment but in subsequent treatments, the dosage may be adjusted to achieve only partial depletion. In one embodiment, the B cell depletion is at least 20%, i.e., 80% or less of CD20 positive or BR3 positive cells remain as compared to the baseline level before treatment. In other embodiments, B cell depletion is 25%, 30%, 40%, 50%, 60%, 70% or greater. Preferably, the B cell depletion is sufficient to halt progression of the disease, more preferably to alleviate the signs and symptoms of the particular disease under treatment, even more preferably to cure the disease.

Publications concerning therapy with Rituximab include: Perotta and Abuel “Response of chronic relapsing ITP of 10 years duration to Rituximab” Abstract # 3360 Blood 10(1)(part 1-2): p. 88B (1998); Stashi et al. “Rituximab chimeric anti-CD20 monoclonal antibody treatment for adults with chronic idiopathic thrombocytopenic purpura” Blood 98(4):952-957 (2001); Matthews, R. “Medical Heretics” New Scientist (7 April, 2001); Leandro et al. “Clinical outcome in 22 patients with rheumatoid arthritis treated with B lymphocyte depletion” Ann Rheum Dis 61:833-888 (2002); Leandro et al. “Lymphocyte depletion in rheumatoid arthritis: early evidence for safety, efficacy and dose response. Arthritis and Rheumatism 44(9): S370 (2001); Leandro et al. “An open study of B lymphocyte depletion in systemic lupus erythematosus”, Arthritis & Rheumatism 46(1):2673-2677 (2002); Edwards and Cambridge “Sustained improvement in rheumatoid arthritis following a protocol designed to deplete B lymphocytes” Rheumatology 40:205-211 (2001); Edwards et al. “B-lymphocyte depletion therapy in rheumatoid arthritis and other autoimmune disorders” Biochem. Soc. Trans. 30(4):824-828 (2002); Edwards et al. “Efficacy and safety of Rituximab, a B-cell targeted chimeric monoclonal antibody: A randomized, placebo controlled trial in patients with rheumatoid arthritis. Arthritis and Rheumatism 46(9): S197 (2002); Levine and Pestronk “IgM antibody-related polyneuropathies: B-cell depletion chemotherapy using Rituximab” Neurology 52: 1701-1704 (1999); DeVita et al. “Efficacy of selective B cell blockade in the treatment of rheumatoid arthritis” Arthritis & Rheum 46:2029-2033 (2002); Hidashida et al. “Treatment of DMARD-Refractory rheumatoid arthritis with rituximab.” Presented at the Annual Scientific Meeting of the American College of Rheumatology; October 24-29; New Orleans, La. 2002; Tuscano, J. “Successful treatment of Infliximab-refractory rheumatoid arthritis with rituximab” Presented at the Annual Scientific Meeting of the American College of Rheumatology; October 24-29; New Orleans, La. 2002.

For therapeutic applications, the compositions of the invention, optionally including CD20 binding antibodies, can be used in combination therapy with, e.g., chemotherapeutic agents, hormones, antiangiogens, radiolabelled compounds, or with surgery, cryotherapy, and/or radiotherapy. The preceding treatment methods can be administered in conjunction with other forms of conventional therapy, either consecutively with, pre- or post-conventional therapy. The polypeptide of this invention will be administered with a therapeutically effective dose of the chemotherapeutic agent. In another embodiment, the polypeptide of this invention is administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

A patient is alleviated or successfully treated of a B cell neoplasm or a B cell regulated autoimmune diseases by the present methods of the invention if there is a measurable improvement in the symptoms or other applicable criteria after administration of the compositions of the invention compared to before treatment. The effect of treatment may be apparent within 3-10 weeks after administration of the compositions of the invention. The applicable criteria for each disease will be well known to the physician of skill in the appropriate art. For example, the physician can monitor the treated patient for clinical, or serologic evidence of disease such as serologic markers of disease, complete blood count including B cell count, and serum immunoglobulin levels. Serum levels of IgG and IgM are reduced in TACI-Fc treated mice. It is expected that human patients responding to immunoadhesins of this invention, anti-CD20 antibody treatment or both would likewise show a reduction in serum IgG and IgM levels. The patient may show observable and/or measurable reduction in or absence of one or more of the following: reduction in the number of cancer cells or absence of the cancer cells; reduction in the tumor size; inhibition (i.e., slow to some extent and preferably stop) of cancer cell infiltration into organs; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. Preferably, after administration of the compositions of the invention, the improvement is at least 20% over the baseline for a particular symptom or criterion taken before treatment by the methods of the invention, more preferably, 25-30%, even more preferably 30-35%, most preferably 40% and above.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission and stable disease. For B cell neoplasms, measurable criteria may include, e.g., time to disease progression, an increase in duration of overall and/or progression-free survival. In the case of leukemia, a bone marrow biopsy can be conducted to determine the degree of remission. Complete remission can be defined as the leukemia cells making up less than 5 percent of all cells found in a patient's bone marrow 30 days following treatment.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D: Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples. Rheumatoid arthritis (RA) is an autoimmune disorder of unknown etiology. Most RA patients suffer a chronic course of disease that, even with therapy, may result in progressive joint destruction, deformity, disability and even premature death. The goals of RA therapy are to prevent or control joint damage, prevent loss of function and decrease pain. Initial therapy of RA usually involves administration of one or more of the following drugs: nonsteroidal anti-inflammatory drugs (NSAIDs), glucocorticoid (via joint injection), and low-dose prednisone. See “Guidelines for the management of rheumatoid arthritis” Arthritis & Rheumatism 46(2): 328-346 (February, 2002). The majority of patients with newly diagnosed RA are started with disease-modifying antirheumatic drug (DMARD) therapy within 3 months of diagnosis. DMARDs commonly used in RA are hydroxycloroquine, sulfasalazine, methotrexate, leflunomide, etanercept, infliximab (plus oral and subcutaneous methrotrexate), azathioprine, D-penicillamine, Gold (oral), Gold (intramuscular), minocycline, cyclosporine, Staphylococcal protein A immunoadsorption.

Because the body produces tumor necrosis factor alpha (TNF) during RA, TNF inhibitors have used for therapy of that disease. Etanercept (ENBREL®) is an injectable drug approved in the US for therapy of active RA. Etanercept binds to TNF and serves to remove most TNF from joints and blood, thereby preventing TNF from promoting inflammation and other symptoms of rheumatoid arthritis. Etanercept is an “immunoadhesin” fusion protein consisting of the extracellular ligand binding portion of the human 75 kD (p75) tumor necrosis factor receptor (TNFR) linked to the Fc portion of a human IgG1. Infliximab, sold under the trade name REMICADE®, is an immune-suppressing drug prescribed to treat RA and Crohn's disease. Infliximab is a chimeric monoclonal antibody that binds to TNF and reduces inflammation in the body by targeting and binding to TNF which produces inflammation.

Adalimumab (HUMIRA™, Abbott Laboratories), previously known as D2E7, is a human monoclonal antibody that binds to TNF and is approved for reducing the signs and symptoms and inhibiting the progression of structural damage in adults with moderately to severely active RA who have had insufficient response to one or more traditional disease modifying DMARDs.

Treatment of rheumatoid arthritis by administering a polypeptide of this invention, optionally in combination with CD20 binding antibodies, can be preformed in conjunction with therapy with one or more of the aforementioned drugs for RA.

For rheumatoid arthritis, for example, measurements for progress in treatment may include the number of swollen and tender joints and the length of morning stiffness. Patients may be examined for how much the joint in the hands and feet have eroded by using X-rays and a scoring system known as the Sharp score. Another scoring system is based on the American College of Rheumatology criteria for assessing response to therapies.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g, baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:

patient's pain assessment by visual analog scale (VAS),
patient's global assessment of disease activity (VAS),
physician's global assessment of disease activity (VAS),
patient's self-assessed disability measured by the Health Assessment Questionnaire, and
acute phase reactants, CRP or ESR.

The ACR 50 and 70 are defined analogously. Preferably, the patient is administered an amount of a polypeptide of this invention effective to achieve at least a score of ACR 20, preferably at least ACR 30, more preferably at least ACR50, even more preferably at least ACR70, most preferably at least ACR 75 and higher. Psoriatic arthritis has unique and distinct radiographic features. For psoriatic arthritis, joint erosion and joint space narrowing can be evaluated by the Sharp score as well. The polypeptides of this invention disclosed herein can be used to prevent the joint damage as well as reduce disease signs and symptoms of the disorder. Yet another aspect of the invention is a method of treating Lupus or SLE by administering to the patient suffering from SLE, a therapeutically effective amount of a polypeptide of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, psoriatic arthritis and Crohn's disease. Treatment success can be determined by validated patient and physician global assessment measuring tools.

For systemic lupus erythematosus, patients can be monitored for levels of antinuclear antibodies and antibodies to double-stranded DNA.

Various medications are used to treat psoriasis; treatment differs directly in relation to disease severity. Patients with a more mild form of psoriasis typically utilize topical treatments, such as topical steroids, anthralin, calcipotriene, clobetasol, and tazarotene, to manage the disease while patients with moderate and severe psoriasis are more likely to employ systemic (methotrexate, retinoids, cyclosporine, PUVA and UVB) therapies. Tars are also used. These therapies have a combination of safety concerns, time consuming regimens, or inconvenient processes of treatment. Furthermore, some require expensive equipment and dedicated space in the office setting. Systemic medications can produce serious side effects, including hypertension, hyperlipidemia, bone marrow suppression, liver disease, kidney disease and gastrointestinal upset. Also, the use of phototherapy can increase the incidence of skin cancers. In addition to the inconvenience and discomfort associated with the use of topical therapies, phototherapy and systemic treatments require cycling patients on and off therapy and monitoring lifetime exposure due to their side effects.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The patient can be measured periodically throughout treatment on the Visual analog scale used to indicate the degree of itching experienced at specific time points.

### Dosing

Depending on the indication to be treated and factors relevant to the dosing that a physician of skill in the field would be familiar with, the polypeptides of the invention will be administered at a dosage that is efficacious for the treatment of that indication while minimizing toxicity and side effects. For the treatment of patients suffering from B-cell neoplasm such as non-Hodgkins lymphoma, in a specific embodiment, the polypeptides of the invention can be administered to a human patient (optionally in combination with an anti-CD20 antibody) at a dosage range of 1 mg/kg to 20 mg/kg body weight, preferably at 2.5 mg/kg to 10 mg/kg. In one embodiment, the anti-CD20 antibody is administered at a dosage of 10 mg/kg or 375 mg/m². For treating NHL, one dosing regimen would be to administer 375 mg/m² of anti-CD20 antibody every other week for 2-4 doses, or one dose of the antibody composition in the first week of treatment, followed by a 2 week interval, then a second dose of the same amount of antibody is administered. Generally, NHL patients receive such treatment once during a year but upon recurrence of the lymphoma, such treatment can be repeated. In the treatment of NHL, the anti-CD20 antibody plus TACI polypeptide therapy can be combined with chemotherapy such as with CHOP. In another embodiment, for the treatment of B cell neoplasms such as CLL or SLL, patients may receive four weekly doses of Rituxan at 375 mg/m² after or before administration with TACI polypeptide with relapsed CLL. For CLL, treatment with the polypeptides of this invention, optionally in combination with anti-CD20 antibodies, can be combined with chemotherapy, for example, with fludarabine and cytoxan.

For treating rheumatoid arthritis, in one embodiment, the Rituxan™ antibody which is a chimeric antibody is administered at 500 mg per dose every other week for a total of 2 doses. A humanized anti-CD20 antibody, e.g., hu2H7v.16 or any other variant of hu 2H7 as disclosed herein, can be administered at less than 500 mg per dose such as at between about 200-500 mg per dose, between about 250 mg-450 mg, or 300-400 mg per dose, for 2-4 doses every other week or every 3rd week.

TACI polypeptide can be administered at a dosage range of 0.5 mg/kg to 10 mg/kg, preferably 1 mg/kg to 5 mg/kg, more preferably, 1.5 mg/kg to 2.5 mg/kg. In one embodiment, TACI-Fc is administered at 5 mg/kg every other day from day 1 to day 12 of treatment. Also contemplated is dosing at about 2-5 mg/kg every 2-3 days for a total of 2-5 doses.

The treatment methods of the invention comprises a combination of concurrently and sequentially administering the anti-CD20 antibody and the polypeptides of this invention (both referred to herein as the drugs). In sequential administration, the drugs can be administered in either order, i.e., the polypeptides of this invention first followed by anti-CD20 antibody. The patient is treated with one drug and monitored for efficacy before treatment with the one drug. For example, if the polypeptides of this invention produces a partial response, treatment can be followed with the anti-CD20 antibody to achieve a full response, and vice versa. For the treatment of autoimmune diseases such as rheumatoid arthritis, if the anti-CD20 antibody is Rituxan and the polypeptides of this invention is an immunadhesin, in one embodiment, the patient in need thereof receives the immunadhesin prior to treatment with Rituxan. Alternatively, the patient can be initially administered both drugs and subsequent dosing can be with only one or the other drug.

To condition the patient to tolerate the drugs and/or to reduce the occurrence of adverse effects such as infusion-related symptoms which arise from the initial and subsequent administrations of the therapeutic compound, the mammal in need thereof can be administered a first or initial conditioning dose of one or both drugs and then administered at least a second therapeutically effective dose of one or both drugs wherein the second and any subsequent doses are higher than the first dose. The first dose serves to condition the mammal to tolerate the higher second therapeutic dose. In this way, the mammal is able to tolerate higher doses of the therapeutic compound than could be administered initially. A “conditioning dose” is a dose which attenuates or reduces the frequency or the severity of first dose adverse side effects associated with administration of a therapeutic compound. The conditioning dose may be a therapeutic dose, a sub-therapeutic dose, a symptomatic dose or a sub-symptomatic dose. A therapeutic dose is a dose which exhibits a therapeutic effect on the patient and a sub-therapeutic dose is a dose which dose not exhibit a therapeutic effect on the patient treated. A symptomatic dose is a dose which induces at least one adverse effect on administration and a sub-symptomatic dose is a dose which does not induce an adverse effect. Some adverse effects are fever, headache, nausea, vomiting, breathing difficulties, myalgia, and chills.

### Route of Administration

The TACI polypeptides, optionally in combination with the anti-CD20 antibodies, are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes. The anti-CD20 antibody will generally be administered by intravenous or subcutaneous administration. The drugs can be administered by the same or different route.

### Articles of Manufacture and Kits

Another embodiment of the invention is an article of manufacture comprising a TACI polypeptide alone or in combination with an anti-CD20 antibody for the treatment of a B cell based malignancy or a B-cell regulated autoimmune disorder disclosed above. In a specific embodiment, the article of manufacture contains the TACI polypeptide and an anti-CD20 antibody, for the treatment of non-Hodgkin's lymphoma.

The article of manufacture comprises at least one container and a label or package insert on or associated with the container. Suitable containers include, for example, bottles, vials, syringes, etc. The containers may be formed from a variety of materials such as glass or plastic. The container holds a composition of the invention which is effective for treating the condition and may have a sterile access port (for example the container may be an intravenous solution bag or a vial having a stopper pierceable by a hypodermic injection needle). At least one active agent in the composition is a polypeptide of this invention. Optionally also included is a CD20 binding antibody of the invention such as Rituxan™ or hu2H7v.16. The label or package insert indicates that the composition is used for treating the particular condition, e.g., non-Hodgkin's lymphoma or rheumatoid arthritis. The label or package insert will further comprise instructions for administering the composition to the patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for B-cell killing assays. As with the article of manufacture, the kit comprises a container and a label or package insert on or associated with the container. The container holds a composition comprising at least polypeptide of the invention and optionally an anti-CD20 antibody. Additional containers may be included that contain, e.g., diluents and buffers, control antibodies. The label or package insert may provide a description of the composition as well as instructions for the intended in vitro or diagnostic use.

### EXAMPLES

### Example 1

### Materials and Methods

### Reagents

Reagents were obtained from the following sources: o-Phenylenediamine dihydrochloride (OPD) (Sigma); Streptavidin peroxidase (POD) (Roche/Boehringer Mannheim); IgG-horseradish peroxidase (HRP) (Jackson Immuno Research Laboratories); Protease Complete (Boehringer Mannheim); anti-M13-HRP (Boehringer Mannheim); sulpho NHS-biotin (Pierce). Murine APRIL, human BAFF, human BCMA-Fc, and human BR3 were purified as described previously (Patel et al., 2004).

### Expression Plasmids

The pRL-TK (Promega) and ELAM-luciferase were described previously (Curr Biol. 2000 Jun. 29; 10(13):785-8).

### TACI_d1d2 Protein Production

DNA coding for residues 21-116 of the TACI extracellular domain was amplified by PCR and subcloned into the pET32a (Novagen) expression vector. This construct served as the template for a second round of PCR that produced an amplicon containing the TACI coding region of interest with a C-terminal His-tag, which was subsequently subcloned into the baculovirus transfer vector pAcGP67B (Pharmingen). The transfer vector was cotransfected with BaculoGold DNA (Novagen) into Sf9 cells and recombinant virus was isolated and amplified to facilitate protein production.

Protein was purified from culture medium harvested by centrifugation after three days of growth of virally infected Hi5 cells at 27° C. 50 mM Tris, pH 8.0, 1 mM NiCl₂, 5 mM CaCl₂, and 1 μM phenylmethylsulfonyl fluoride were added to the culture medium, the pH was adjusted to 7.6, and the medium filtered prior to loading onto Ni-NTA resin pre-equilibrated in 20 mM Tris buffer pH 8.0, 400 mM NaCl, and 10 mM imidazole. After extensive washing, protein was eluted with the same buffer containing 400 mM imidazole. Relevant fractions were pooled and the protein concentrated prior to loading on a Superdex-75 column equilibrated in 20 mM sodium phosphate, pH 5.5, 400 mM NaCl. TACI_d1d2 eluted from the column with the approximate retention time of the monomeric species. N-terminal sequencing and mass spectrometry confirmed the proper identity of the purified protein.

### TACI_d2 Protein Production

The second cysteine-rich domain (CRD2, residues 68-109) of TACI was subcloned into the pET32a expression vector (Novagen), creating a fusion with an N-terminal thioredoxin (TRX)-His-tag followed by an enterokinase protease site. E. coli BL21(DE3) cultures harboring the expression plasmid were grown at 37° C. to mid-log phase (A₆₀₀=0.7) in rich medium (LB) supplemented with 50 ug/mL carbenicillin for unlabeled protein, or M9 minimal media supplemented with 50 u/mL carbenicillin with 1.0 g of ¹⁵NH₄Cl and 4.0 g ¹²C₆-glucose (uniform ¹⁵N-labeled), 1.0 g of ¹⁵NH₄Cl and 4.0 g C₆-glucose (uniform ¹⁵N, ¹³C-labeled), or 1.0 g of ¹⁵NH₄Cl and 3.4 g ¹²C₆-glucose and 0.6 g ¹³C₆-glucose (uniform ¹⁵N—, fractional ¹³C-labeled) per liter. Protein expression was induced by transferring cultures to 22° C., and adding IPTG to a final concentration of 1 mM. Cells were harvested after 3 hours of additional growth and lysed with a microfluidizer. TRX-TACI_d2 was purified over a Ni-NTA Superflow (Qiagen) column. and eluted with 50 mM Tris, pH 7.5, 0.5 M NaCl, 500 mM imidazole. The fusion protein was cleaved with thrombin overnight at 4° C. TACI_d2 was purified by preparative C-18 reverse-phase HPLC using a 10-70% acetonitrile (0.1% trifluoroacetic acid) gradient. Fractions containing purified TACI_d2 were lyophilized, and later resuspended in water and dialyzed against 50 mM sodium phosphate, pH 7.2, 50 mM NaCl. The identity of the purified protein was verified by N-terminal sequencing and mass spectrometry. In addition to TACI_d2 (residues 68-109), the construct also contained four additional residues (GSPW) at the N-terminus from the expression vector.

### TACI_d1 Protein Production

DNA coding for the first CRD of TACI (residues 32-67) was subcloned into the pET32a (Novagen) expression vector creating a fusion with an N-terminal thioredoxin, followed by a His-tag and thrombin cleavage site. Origami (DE3) pLysS E. coli cells (Novagen) harboring the plasmid were grown at room temperature and protein expression was induced with IPTG. TACI_d1 was purified over a Ni-NTA column (Qiagen) and eluted with an imidazole gradient The fusion was cleaved with thrombin and the concentrated cleavage product was dialyzed into PBS. TACI_d1 was then purified over a Superdex S75 gel filtration column, dialyzed into 25 mM MES, pH 5.5, and further purified over a monoS (Pharmacia) cation exchange column. The purified protein was then dialyzed into PBS. N-terminal sequencing and mass spectrometry confirmed the identity of the purified protein. Reverse-phase analytical HPLC analysis using a C18 column (Vydak) revealed several peaks with slightly different retention times, but identical mass, suggesting that more than one disulfide-bonded isomer was present in the TACI_d1 sample. 2D NMR analysis indicated the presence of two major forms, with the dominant form showing significant chemical shift dispersion and evidence of being folded in solution.

### Competitive Binding Monitored by Surface Plasmon Resonance

Surface plasmon resonance (SPR) measurements on a BIAcore 3000 instrument (Pharmacia Biosensor) were used to measure binding affinities of receptors to immobilized APRIL and BAFF by competition binding. Competition experiments were used because direct binding experiments at low-density ligand immobilization (˜400 RU) did not produce results with sufficient signal to noise ratios for analysis. In these experiments, receptors in solution competed for binding of ligand in solution at room temperature with immobilized BCMA-Fc. BCMA-Fc was coupled to flow cell 2 of a CM4 sensor chip, using the amine coupling protocol supplied by the manufacturer, at a high density (4500 resonance units) so that the initial rate of binding was linearly dependent on the concentration of free ligand. In all experiments flow cell 1 was ethanolamine-blocked and used as the reference cell. Washing with 10 mM HCl regenerated flow cells between sample injections. In order to determine the ligand concentration necessary to obtain the optimal for competition experiments, a preliminary binding study was performed for each ligand where two-fold dilutions of the ligand ranging from 400 nM to 6.25 nM were injected over the BCMA-Fc surface. The concentration of ligand that provided a slope of about 1 for the initial on-rate of the observed sensorgrams was selected as the fixed concentration to be used in the competition experiments. In all experiments where BAFE was the ligand, the buffer used was composed of 10 mM HEPES, pH 7.2, 150 mM NaCl, and 0.005% Tween-20. In all experiments where TRX-APRIL was the ligand selected, the buffer was composed of 20 mM Tris-HCl, pH 8.0, 200 mM NaCl, and 0.005% Tween-20. For the competition experiment, the selected fixed ligand concentrations (12.5 nM BAFF; 50 nM TRX-APRIL) were incubated with competing receptor in two-fold dilutions starting at 100× the expected IC₅₀, as well as no competing receptor as a control, for one hour at room temperature to allow samples to reach equilibrium. Samples were then passed over the BCMA-Fc-coupled surface. A linear fit of the initial on-rate of the observed sensorgrams was used to calculate the binding rate (V). The IC₅₀ values were calculated using a four-parameter curve fit (y=((m1−m4)/(1+(m0/m3)̂m2))+m4) of the initial binding rates for the receptor/control (Vᵢ/Vₒ) as a function of receptor concentration.

### NMR Spectroscopy of TACI_d2

NMR samples typically contained 0.8-1.2 mM protein, 50 mM sodium phosphate, pH 7.2, 50 mM NaCl, 0.1 mM sodium azide, and 50 uM 1,4-dioxane as an internal chemical shift reference standard in 90% H₂O, 10% D₂O. A “100% D₂O” sample was prepared by lyophilization and resuspension in 99.995% D₂O. NMR spectra were acquired at 17° C. on a Bruker DRX600 spectrometer equipped with a triple resonance cryoprobe. All NMR data were processed using FELIX (version 2000.1; Accelrys, San Diego, Calif.) and analyzed using the program Sparky (version 3.11, Goddard & Kneller, University of California, San Francisco). Backbone resonance assignments of TACI_d2 were obtained from the following double and triple resonance experiments in H₂O solution, as described (Cavanagh et al., 1995): ¹H-5N three-dimensional (3D) NOESY-HSQC, 3D ¹H-¹⁵N TOCSY-HSQC, 3D HNCA, and 3D CBCA(CO)NH. Side chain resonance assignments were obtained from a 3D HCCH-TOCSY spectrum measured in D₂O solution. Stereospecific assignments of leucine methyl groups were obtained from a ¹H-¹³C HSQC spectrum of a 15% ¹³C-labeled sample (Neri et al., 1989). Backbone dynamics were investigated by analyzing the steady state ¹H-¹⁵N-NOE as described (Gordon et al., 2003; Skelton et al., 1993).

Distance restraints were obtained from analysis of the following NOESY spectra: 3D ¹H-¹⁵N-edited NOESY (200 ms mixing time) measured in H₂O, 3D ¹³C-edited NOESY (150 ms mixing time) measured in D₂O, and 2D ¹H-¹H NOESY (150 ms mixing time) measured in D₂O, NOE peaks were picked manually, and NOE assignments were obtained using the automated NOE assignment program CANDID (Herrmann et al., 2002), followed by several rounds of structure calculation and manual restraint checking. Dihedral angle restraints were obtained from analysis of 3D ¹⁵N-¹H HNHB and 3D ¹³C-edited NOESY-HSQC (50 ms mixing time) spectra. Additional loose backbone dihedral angle restraints were obtained from analysis of backbone chemical shifts with the program TALOS (Cornilescu et al., 1999). Dihedral restraints were applied for good fits to the chemical shifts (as defined in the manual) with the allowed range being the greater of ±30° (for Φ) ±40° (for Ψ) or three times the uncertainty estimated by TALOS. The final structures were calculated using the program CNX (version 2002; Accelrys, San Diego, Calif.). 100 structures were calculated using torsion angle dynamics followed by Cartesian dynamics and minimization. The 20 structures with the lowest restraint violation energy were chosen to represent the solution structure. Details of the input restraints and structural statistics are presented in Table 2.

TABLE 2
Structural Statistics for the Solution Structure of TACI_d2

| Parameter | Ensemble |
|---|---|
| Input restraints | |
| NOE Total | |
| Intra-residue; Sequential; Medium-range; Long- | 466; 124; 95; 123; 124 |
| range | |
| Dihedral Angles Total | |
| Φ; ψ; ω^{a}; χ₁^{b}; χ₃^{c} | 188; 40; 27; 90; 25; 6 |
| Violations | |
| RMSD from experimental restraints | |
| NOE-distance (Å) | 0.0045 ± .0009 |
| Dihedral (°) | 0.18 ± 0.04 |
| NOE distance violations | |
| Number > 0.01 Å | 12.4 ± 2.4 |
| Number > 0.1 Å | 0.0 |
| Mean maximum violations (Å) | 0.04 ± 0.01 |
| Dihedral violations | |
| Number > 0.1° | 7.8 ± 2.3 |
| Mean maximum violations (°) | 0.72 ± 0.24 |
| RMSD from idealized geometry | |
| Bonds (Å) | 0.0009 ± 0.0001 |
| Angles (°) | 0.27 ± 0.01 |
| Impropers (°) | 0.12 ± 0.01 |
| Energies | |
| Energy components (kcal · mol.⁻¹) | |
| NOE (466) | 0.36 ± 0.12 |
| CDIH (188) | 0.09 ± 0.06 |
| Bonds | 0.70 ± 0.18 |
| Angles | 15.34 ± 0.96 |
| Impropers | 0.88 ± 0.15 |
| Vanb der Waal's | 8.06 ± 1.74 |
| Stereochemistry | |
| Ramachandran (%) | |
| Favored; Allowed; Generous; Disallowed | 66.8; 31.6; 1.6; 0.0 |
| Structural precision | |
| Mean RMSD to mean structure (Å) | |
| Backbone | |
| Residues 76-104 | 0.55 ± 0.08 |
| Residues 76-88 | 0.28 ± 0.05 |
| Residues 89-104 | 0.25 ± 0.10 |
| Heavy | |
| Residues 76-104 | 0.92 ± 0.10 |
| Residues 76-88 | 0.81 ± 0.13 |
| Residues 89-104 | 0.61 ± 0.10 |

^{a}Amide groups were restained to be within 10° of planarity.
^{b}Chi-1 angles were restrained to a particular staggered rotamer ± 30°.
^{c}In the case of cysteine residues for which a unique rotamer could not be defined by the HNHB and NOE data, two or three overlapping restraints were applied to limit the side chain to occupy staggered conformations only. Chi-3 angle restraints were restrained to ±90 ± 20° by using two overlapping dihedral angle restraints.

One of the differences between the NMR ensemble of TACI_d2 and the crystal structure of the same domain bound to APRIL is the conformation of the C-terminal disulfide (Cys93/Cys104). Thus, a separate set of 100 structures was calculated for comparison to the TACI_d2 crystal structure using identical input restraints with the exception that the dihedral angle restraints χ₁, χ₂ and χ₃ for the disulfide bonds were forced to adopt the conformation observed in the crystal structure (−60±20° for χ₁, −60°+20° for χ₂ and −90±20° for χ₃). Forcing the disulfide bonds to adopt the geometry seen in the crystal structure did not introduce significant NOE or dihedral angle restraint violation energy (rmsd values 0.008 and 0.36, respectively), and resulted in the C-terminal disulfide bond adopting a conformation identical to that of the crystal structure without noticeable changes in the backbone conformation (0.58±0.09 Å backbone rmsd for residues 76-104 to mean structure from previous round). Thus the difference in conformation seen for the Cys93/Cys104 disulfide bond may result from the lack of experimental dihedral angle restraints and the imprecise nature of the NOE restraints in this region.

### Production and Crystallization of APRIL-Receptor Complexes

Murine APRIL was expressed and purified as described previously (Patel et al., 2004; Wallweber et al., 2004) in 20 mM CAPS pH 9.7, 400 mM NaCl. APRIL and TACI_d2 were mixed at a 1:3 molar ratio and the complex was purified over a Superdex-75 sizing column in 20 mM CAPS, pH 9.7, 400 mM NaCl and concentrated to 1 mg/ml. Crystals of the APRIL-TACI_d2 complex were grown by vapor diffusion at 19° C. from sitting drops containing 1 μl protein and 1 μl reservoir solution which consisted of 70% MPD, 0.1 M Hepes, pH 7.5.

### Crystallography

APRIL-TACI_d2 crystals were cryo-cooled without any additional cryo-protectant. An initial APRIL-TACI_d2 dataset was collected to 2.7 Å resolution in-house on a MAR345 detector using a Rigaku rotating anode source. A 1.9 Å resolution APRIL-TACI_d2 dataset was collected at beamline 19BM at the APS. Data processing was performed with the HKL suite of programs (Otwinowski and Minor, 1997). Data processing statistics and examination of systematic absences indicated that the APRIL-TACI_d2 crystals belonged to space group P2₁2₁2₁. Calculation of the Matthew's coefficient indicated that the asymmetric units contained one APRIL trimer and three copies of receptor.

The APRIL-TACI_d2 structure was solved using the 2.7 Å dataset by molecular replacement using the 2.3 Å structure of APRIL alone as the search model (Wallweber et al., 2004). Using the program AMoRe with all data from 8-4 Å, a solution was found with a correlation coefficient of 50.4% and an initial R_{factor} of 43%. Following adjustments to the conformation of the APRIL EF and CD loops and refinement of the APRIL portion of the structure, good density was observed for all three copies of TACI_d2. Refinement was performed with the program REFMAC5 (CCP4, 1994) using the 1.9 Å data set and included TLS refinement and NCS restraints imposed separately on APRIL and TACI_d2 resulting in an R_{factor} and R_{free} of 18.3% and 21.5%, respectively (Table 3).

TABLE 3
Data Collection and Refinement Statistics For
APRIL-Receptor Complexes

| | APRIL-TACI_d2 | APRIL-BCMA |
|---|---|---|
| Data Collection | | |
| Space Group | P2₁2₁2₁ | P6₁ |
| Resolution (Å) | 50-1.90 (1.97-1.90)^{a} | 50-2.35 (2.43-2.35)^{a} |
| Unit cell constants (Å) | a = 59 b = 92 c = 102 | a = 114 c = 91 |
| R_{sym}^{b} | 0.086 (0.347)^{a} | 0.067 (0.425)^{a} |
| No. observations | 289,762 | 266,614 |
| Unique reflections | 44,579 | 28,292 |
| Completeness (%) | 99.6 (100)^{a} | 99.9 (100)^{a} |
| Asymmetric Unit | 1 APRIL trimer; | 1 APRIL trimer; |
| | 3 TACI_d2 | 3 BCMA |
| Refinement | | |
| Resolution (Å) | 30-1.90 | 30-2.35 |
| Final R^{c}, R_{free} (%) | 16.7, 20.3 | 17.8/21.3% |
| No. solvent atoms | 131 | 37 |
| Rmsd bonds (Å) | 0.009 | 0.010 |
| Rmsd angles (°) | 1.2 | 1.2 |
| Rmsd bonded Bs (Å²) | 3.0 | 2.8 |
| Ramachandran Plot (%)^{d} | 92.3; 7.7; 0; 0 | 91.5; 8.3; 0.2; 0 |

^{a}Numbers in parentheses refer to tbe highest resolution shell.
^{b}R_{sym} = Σ|I − <I>|/ΣI. <I> is the average intensity of symmetry related observations of a unique reflection.
^{c}R = Σ|Fₒ− F_{c}|/ΣFₒ. R_{free} is calculated as R, but for 10% of the reflections excluded from all refinement. The R_{free} set was chosen in thin shells due to the 3-fold non-crystallographic symmetry
^{d}Percentage of residues in the most favored, additionally allowed, generously allowed, and disallowed regions of a Ramachandran plot.

The pdb codes for the taci-april and bcma-april complexes are 1xu1 and 1xu2 respectively.

### Example 2

### shortTACI can Mediate NF-κB Activation by either APRIL or BAFF

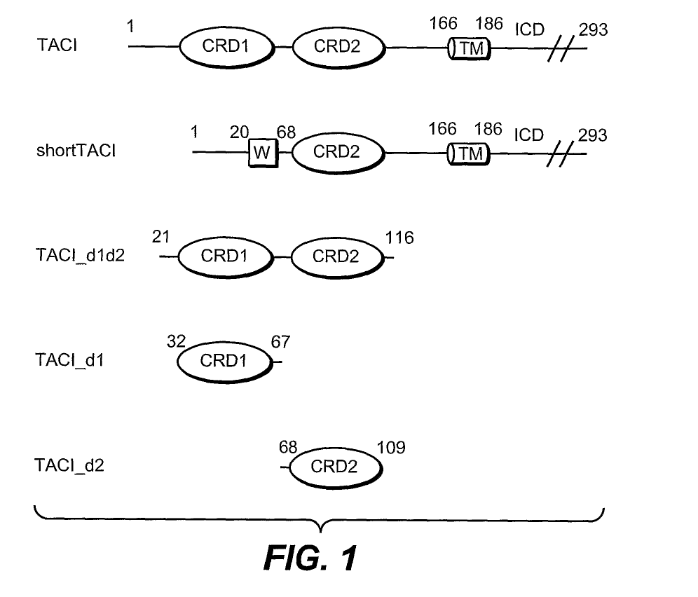

We have shown that stimulation of TACI by its ligands APRIL and BAFF can lead to activation of nuclear factor-κB (NF-κB) in vitro (Marsters et al., 2001). We have also previously shown that there is a splice variant of TACI wherein exon 2, which encodes the first CRD in the extracellular region, has been replaced by a single residue. The polypeptide generated by this alternative splicing event (shortTACI) contains the first 20 residues of TACI, one tryptophan residue in place of 47 residues that encode CRD1, then the rest of the protein including CRD2, the transmembrane, and the intracellular regions (FIG. 1) (Yan et al., 2001a; Yan et al., 2000).

We proceeded to examine whether shortTACI was capable of mediating NF-κB activation by either APRIL or BAFF. Human 293T cells were co-transfected with the indicated amounts of expression plasmids along with 250 ng of ELAM-luciferase reporter gene plasmid and 25 ng pRL-TK. 20 hr after transfection, reporter gene activity was determined with the dual-luciferase reporter assay system (Promega).

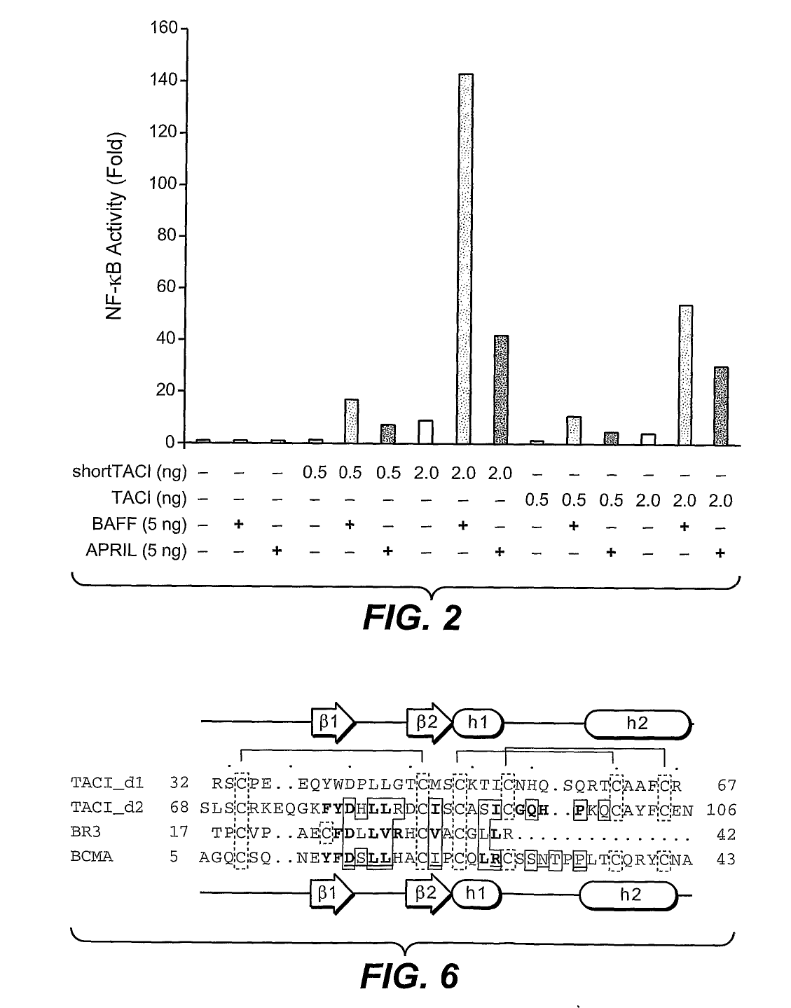

FIG. 2 shows that shortTACI is capable of mediating NF-κB activation by either APRIL or BAFF. The assay depends on co-transfection of the ligand and receptor, hence the extent of signaling observed will depend in part on the relative transfection efficiencies for each gene. Therefore, quantitative comparisons cannot be made from this experiment to indicate the relative effectiveness of each ligand in signaling through each form of the receptor. However, the fact that NF-KB activation can be observed in cells transfected with shortTACI indicates that CRD1 is not required for ligand-dependent NF-KB activation (FIG. 2).

### Example 3

### TACI_d2 is Sufficient for High-Affinity Ligand Binding

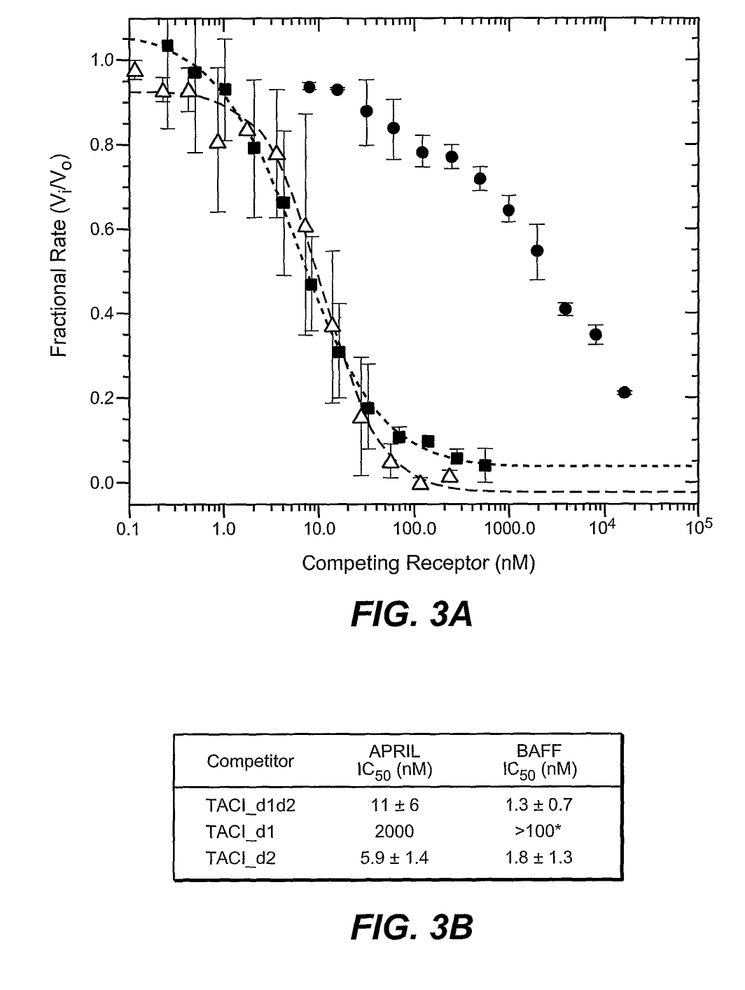

Given that shortTACI containing only a CRD2 was capable of ligand-dependent signaling, the abilities of the individual CRDs of TACI (TACI_d1, TACI_d2) and a construct containing both TACI CRDs (TACI_d1d2) were evaluated for their abilities to bind APRIL or BAFF (FIG. 1). By surface plasmon resonance competition experiments, the 42-residue TACI_d2 fragment was found to have high affinity for both APRIL and BAFF (IC₅₀=6 and 2 nM, respectively; FIG. 3). Moreover, the addition of CRD1 (in the context of the TACI_d1d2 fragment) did not confer additional affinity over that measured for TACI_d2 alone for either ligand. In contrast, the affinity of TACI_d1 was substantially weaker than that of TACI_d2, with IC₅₀ values in the micromolar range, for both APRIL and BAFF (FIG. 3). Competitive ELISA assays confirmed that TACI_d2 is sufficient for high-affinity binding to both ligands, with no improvement in binding with TACI_d1d2 (data not shown).

These results are consistent with those reported previously, where Kim et al. (2003) found that each domain of TACI was capable of binding BAFF through its respective DxL motif (when measured in the context of Fc-fusion proteins), and that mutation of both DxL motifs in full-length TACI was required to eliminate BAFF-binding as detected qualitatively by co-immunoprecipitation. Our interpretation differs from Kim and coworkers (2003), however, due to the finding that TACI_d2 has much higher ligand-binding affinity than TACI_d1. Therefore, in the context of the full-length receptor, these data suggest that the membrane-proximal CRD2 will occupy the DxL-receptor-binding site on the ligand, with CRD1 providing minimal additional binding energy.

### Example 4

### Solution Structure of TACI_d2

The solution structure of TACI_d2 was determined by NMR spectroscopy as described above. The ensemble of the 20 structures of TACI_d2 having the lowest restraint violation energy shows a well-defined core between residues 76105 (0.52±0.08 Å average rmsd to the mean coordinates for N, Cα, C′ backbone atoms) with residues at the N- and C-termini being poorly defined. ¹H-¹⁵N heteronuclear NOE values indicate that the residues at the extreme termini (residues 64-70 and 106-109) appear to be highly flexible on the ps-ns time scale, while residues 71-75 do not exhibit such motions (Table 4, below). The disorder of residues 71-75 in the ensemble is due to a lack of restraints to define this region, and may be due to conformational heterogeneity on a μs-ms time scale. This region also adopts very different conformations in the three TACI_d2 chains present in the asymmetric unit of the APRIL-TACI_d2 crystal structure.

TABLE 4
Summary of the NMR experiments used to characterize the solution structure of
TACI_d2

| | | | | | SW | Points | τₘ | Offset |
|---|---|---|---|---|---|---|---|---|
| Experiment | SF (MHz) | Dim. | Nuc. | NS | (Hz) | (complex) | (ms) | (ppm) |
| ¹H-¹⁵N HSQC | 600 | 1 | N | 4 | 1275.5 | 256* | | 118.0 |
| | | 2 | HN | | 8620 | 4096* | | 4.92 |
| ¹H-¹³C HSQC | 500 | 1 | C | 4 | 4032 | 512* | | 32.4 |
| | | 2 | H | | 6250 | 2048* | | 4.86 |
| TOCSY- | 600 | 1 | H | 16 | 6024 | 128* | 69 | 4.92 |
| HSQC | | 2 | N | | 1275.5 | 32* | | 118.0 |
| | | 3 | HN | | 8620 | 1024* | | 4.92 |
| NOESY- | 600 | 1 | H | 16 | 6024 | 128* | 200 | 4.92 |
| HSQC | | 2 | N | | 1275.5 | 32* | | 118.0 |
| | | 3 | HN | | 8620 | 1024* | | 4.92 |
| HNHB | 600 | 1 | HB | 32 | 5760 | 90* | | 4.92 |
| | | 2 | N | | 1275.5 | 32* | | 118.0 |
| | | 3 | HN | | 8620 | 1024* | | 4.92 |
| HNCA | 600 | 1 | CA | 16 | 4237.3 | 64* | | 53.4 |
| | | 2 | N | | 1216.5 | 32* | | 119.3 |
| | | 3 | HN | | 6250 | 1024* | | 4.93 |
| CBCA(CO)NH | 600 | 1 | CAB | 16 | 9058 | 29* | | 44.4 |
| | | 2 | N | | 1340 | 30* | | 119.3 |
| | | 3 | HN | | 6250 | 1024* | | 4.93 |
| HCCH- | 600 | 1 | H | 8 | 4202 | 128* | 12 | 4.93 |
| TOCSY | | 2 | C | | 4854 | 42* | | 32.9 |
| | | 3 | H | | 6250 | 2048* | | 4.93 |
| 13C-edited | 600 | 1 | H | 16 | 5768 | 128* | 50, | 4.93 |
| NOESY | | 2 | C | | 4930 | 32* | 150 | 32.9 |
| | | 3 | H | | 6250 | 1024* | | 4.93 |
| 1H-1H | 600 | 1 | H | 96 | 5763 | 400* | 200 | 4.93 |
| NOESY | | 2 | H | | 6250 | 4096* | | 4.93 |
| HN-NOE | 500 | 1 | H | 16 | 6510 | 2048* | | 4.92 |
| | | 2 | N | | 1014.2 | 128* | | 119.3 |

The overall fold of TACI_d2 is similar to that previously observed for the related TNF receptor BCMA (Liu et al., 2003), and consists of two sub-modules: an N-terminal strand connected to a beta-hairpin (residues 76-88) with a Type I reverse turn containing the conserved DxL motif, followed by a short helix-loop-helix sub-module consisting of a turn of 3₁₀-helix (h1, residues 89-93), a four-residue loop, and a C-terminal 3₁₀-helix (h2, residues 98-105). The disulfide-bonding pattern is also similar to that of BCMA: one disulfide bond (Cys71/Cys86) connects the N-terminus to the beta-hairpin, and two disulfide bonds (Cys89/Cys100, Cys93/Cys104) connect h1 and h2.

The backbone of the conserved DxL loop is very well defined (0.28±0.08 Å backbone rmsd for residues 76-88) and superimposes well with that of both BCMA (rmsd 0.53±0.001 Å for residues 4-16 of the eight copies of BCMA in pdb entry 1 OQD (Liu et al., 2003)) and BR3 (rmsd 0.89 Å for residues 22-34 of the 1 OSX representative structure; (Gordon et al., 2003)) (Figure not shown). The aromatic side chain of Tyr 79 is well ordered in the ensemble, and is positioned above the hairpin. An equivalent aromatic residue in BCMA and BR3 also occupies this non-hydrogen bonding position (Phe14 in BCMA and Phe25 in BR3). Energetic studies of hairpin stability have suggested that a bulky hydrophobic or aromatic side chain at this position stabilizes the hairpin conformation (Cochran et al., 2001; Skelton et al., 2003). There is a well-ordered hydrophobic core formed by the side chains of Phe78, Ile87, Ile92, and Pro97. The side chain of His96 is also well defined and packs into the interior of the C-terminal sub-module.

A superposition of TACI_d2, BCMA, and BR3 reveals remarkable similarity in the structure of the DxL hairpin in the N-terminal sub-module of the domain. Significant differences are apparent, however, in the C-terminal sub-module; this region shows different relative orientations for the helices and loop between TACI_d2 and BCMA, and is essentially missing in BR3. For example, while the TACI_d2 and BCMA hairpins superimpose well, with a backbone rmsd of 0.31 Å² (residues 77-88 and 12-23, respectively), the overall backbone rmsd for the domain is 1.5 Å. Importantly, these differences appear to be a property of the different receptors themselves, and not a product of a ligand-induced conformational change, given that the solution structure of free TACI_d2 and the crystal structure of TACI_d2 in complex with APRIL are essentially the same in this region, as are the structures of BCMA in complex with both BAFF and APRIL. Such differences in domain structure indicate that while all three receptors can interact with their respective ligands in a similar fashion through their DxL motifs, the interactions through their C-terminal sub-modules will differ and likely will dictate the relative affinity and specificity among ligand-receptor pairs within the APRIL/BAFF family. For example, Arg27 in BCMA packs against the same region of APRIL as Pro97 in TACI_d2, despite the fact that they are offset in a primary sequence alignment by five residues (FIG. 6). Furthermore, Gln95 in TACI_d2 makes extensive contacts with APRIL and yet has no counterpart in BCMA.

### Example 5

### Structure of APRIL Bound to TACI_d2

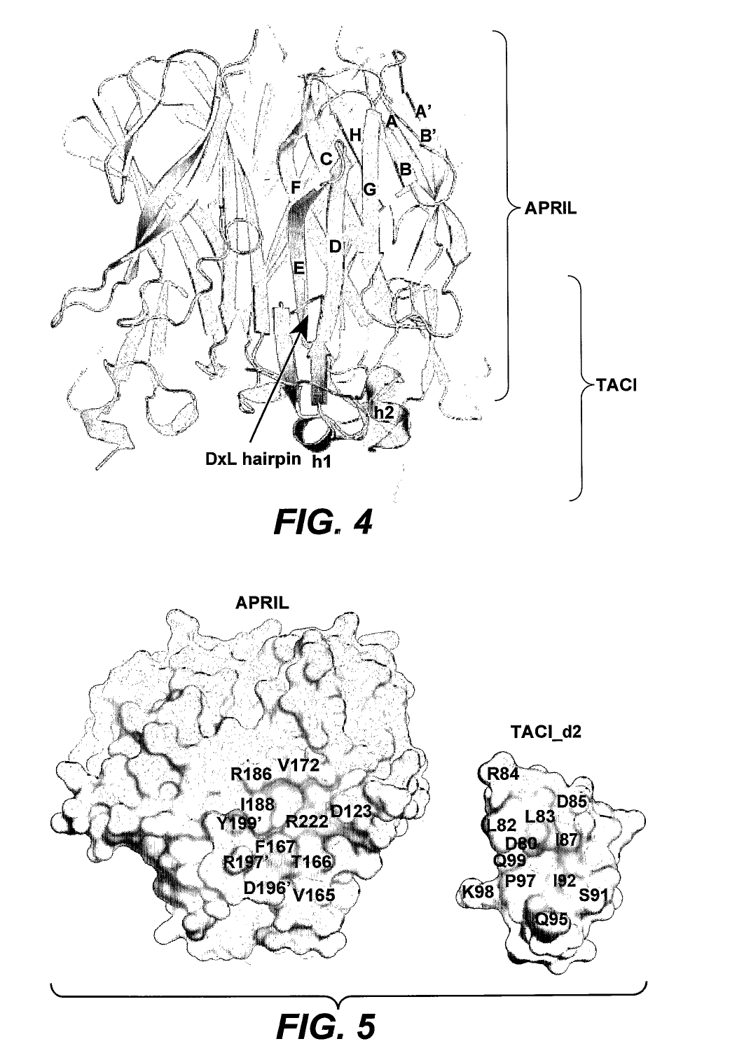

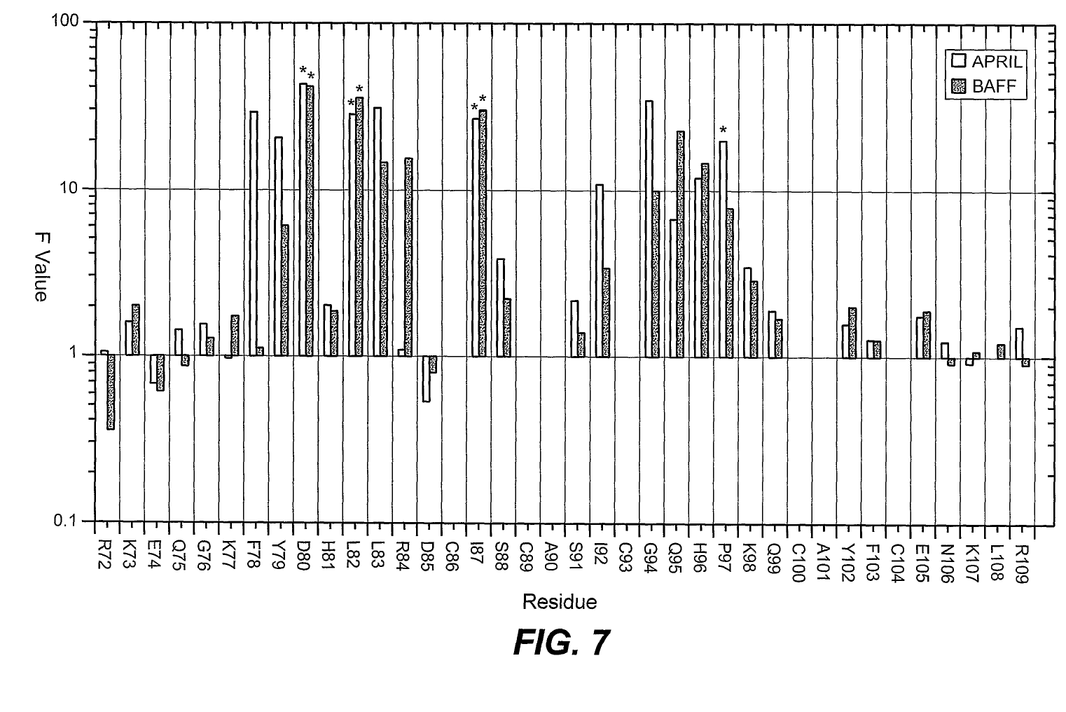

The crystal structure of APRIL in complex with TACI_d2 was solved at 1.9 Å resolution by molecular replacement using the structure of APRIL as a search model (FIG. 4) (Wallweber et al., 2004). The structure of the APRIL component of the complex is very similar to the structure of free APRIL, except that several loops (AA′, CD, and EF) are ordered in the complex, that were either disordered or only marginally ordered in structures of free APRIL (Wallweber et al., 2004). The bound structure of TACI_d2 is similar to the NMR structure (backbone rmsd of the three chains in the asymmetric unit to the mean NMR structure is 0.74±0.06 for residues 76-104). However, the h2 helix is longer in two of the crystallographic chains, and Tyr102 is no longer packed against the rest of the C-terminal subdomain as it is in the NMR ensemble.

In general terms, TACI_d2 binds APRIL in a similar manner as the homologous receptor, BCMA, binds BAFF (Liu et al., 2003). The DxL motif forms a hydrophobic ridge with the two leucine residues (82 and 83) at the tip of the DxL turn nestled in a hydrophobic pocket on APRIL that is ringed by APRIL residues Phe167, Val172, Arg186, Ile188, and Arg222. This pocket is pre-formed at the backbone level, showing little change from the structure of APRIL alone, except that the side chains of Phe167, Thr168, Arg186, and Arg222 are more ordered in the complex. The first helix of TACI, h1, contacts APRIL residues 194-197 in the EF loop. The receptor h1-h2 loop contacts four loops on APRIL (EF, CD, GH, and AA′).

The APRIL-binding surface on TACI_d2 encompasses the entire concave surface defined by mutagenesis; approximately 1700 Å² are buried in this extensive interface (FIG. 5, Table 5, below). Of the residues identified to be functionally important, Leu82, Leu83, and Ile87 in the DxL hairpin, Ile92 in h1, and Gln95 and Pro97 in the h1-h2 loop form a predominantly hydrophobic surface that interacts with APRIL. Of these residues, only Gln95 makes extensive hydrogen bonds. The backbone carbonyl of Gln95 forms a hydrogen bond to the backbone amide of Phe167, while the side chain carbonyl forms hydrogen bonds to the guanidinium moiety of Arg197′ in the EF loop from an adjacent protomer. Furthermore, the side chain amide group of Gln95 forms hydrogen bonds to the carbonyls of Thr165 in the APRIL CD loop and Met191 in the EF loop. This network of hydrogen bonds likely contributes to stabilizing the conformation of the APRIL CD and EF loops which are poorly ordered in the absence of receptor. In contrast, receptor residues Tyr79 and His96 probably contribute to stabilizing the TACI_d2 fold as neither interacts directly with APRIL. Phe78 may have both structural and functional roles: it does not bury significant surface area (3 Å²), but helps position Phe167 of APRIL, as well as restrict the relative orientation of the two sub-modules of TACI_d2.

Table 5 shows the average percent buried surface area per residue for the APRIL-TACI interface. The average and standard deviation are calculated for the 3 interfaces in the trimer. The April residues with a are from the second monomer in the monomer-monomer interface.

TABLE 5

| | | Standard |
|---|---|---|
| | Average % buried surface area | Deviation |
| APRIL Residue | | |
| Lys 119 | 0.2 | 0.3 |
| Ala 120 | 14.2 | 9.6 |
| Asp 121 | 5.0 | 5.3 |
| Ser 122 | 13.5 | 1.6 |
| Asp 123 | 49.9 | 2.7 |
| Val 165 | 45.7 | 1.4 |
| Thr 166 | 67.9 | 1.1 |
| Phe 167 | 100.0 | 0.0 |
| Thr 168 | 100.0 | 0.0 |
| Met 169 | 94.4 | 9.6 |
| Gly 170 | 100.0 | 0.0 |
| Gln 171 | 100.0 | 0.0 |
| Val 172 | 76.9 | 3.1 |
| Arg 181 | 4.7 | 4.6 |
| Thr 183 | 35.6 | 4.5 |
| Arg 186 | 91.1 | 3.2 |
| Cys 187 | 66.7 | 57.7 |
| Ile 188 | 95.7 | 4.9 |
| Met 191 | 37.6 | 18.2 |
| Ser 193 | 22.5 | 2.5 |
| Pro 221 | 84.1 | 3.0 |
| Arg 222 | 93.8 | 0.2 |
| Ala 223 | 66.0 | 7.4 |
| Asn 224 | 12.4 | 3.2 |
| Leu 161′ | 3.0 | 5.2 |
| His 163′ | 7.6 | 4.5 |
| Asp 194′ | 19.1 | 7.5 |
| Asp 196′ | 59.5 | 16.8 |
| Arg 197′ | 46.2 | 24.9 |
| Tyr 199′ | 83.5 | 8.8 |
| His 232′ | 40.2 | 1.1 |
| TACI Residues | | |
| Lys 73 | 2.2 | 3.8 |
| Phe 78 | 19.9 | 6.6 |
| Asp 80 | 100.0 | 0.0 |
| His 81 | 20.8 | 1.2 |
| Leu 82 | 100.0 | 0.0 |
| Leu 83 | 88.4 | 3.1 |
| Arg 84 | 54.0 | 5.2 |
| Asp 85 | 29.9 | 13.3 |
| Cys 86 | 4.2 | 5.7 |
| Ile 87 | 87.9 | 18.4 |
| Ser 88 | 9.0 | 8.1 |
| Ser 91 | 64.1 | 1.8 |
| Ile 92 | 96.3 | 6.4 |
| Cys 93 | 7.9 | 6.9 |
| Gly 94 | 22.1 | 2.5 |
| Gln 95 | 93.1 | 0.6 |
| His 96 | 13.9 | 0.9 |
| Pro 97 | 85.9 | 1.6 |
| Lys 98 | 48.0 | 3.0 |
| Gln 99 | 67.7 | 15.7 |

The APRIL-receptor interfaces, although similar to BAFF-BR3, make significant contacts beyond those mediated through the DxL motif. In BAFF-BR3, the DxL hairpin makes the majority of receptor contacts (˜75% of the buried surface area contributed by the receptor)(Kim et al., 2003; Liu et al., 2003); whereas in both the APRIL-TACI and APRIL-BCMA complexes, the receptor DxL hairpin contributes only ˜50% of the total buried surface area. See Table 5, above, for APRIL-TACI interface. Docking BR3 onto APRIL shows that the BR3 hairpin could be accommodated readily with no steric clashes and result in an interface of ˜1000 Å² (80% from the DxL hairpin), yet BR3 does not bind APRIL. Instead, APRIL seems to require additional contacts from other portions of the receptor in order to form high-affinity interactions. The minimal TNFR domain of BR3 does not contain a second sub-module, and hence cannot provide these contacts, likely accounting for the lack of binding between BR3 and APRIL.

TACI_d2 differs from multi-domain TNFR by using most of its CRD surface to contact ligand. In the case of TNFR1 and DR5 in complex with their respective ligands (Banner et al., 1993; Cha et al., 2000; Hymowitz et al., 1999; Mongkolsapaya et al., 1999), the majority of the ligand-binding interactions stem from one loop from each of two adjacent CRDs (analogous to the BAFF/APRIL receptor hairpin, although differing in length and conformation), and both CRDs are required for ligand-binding (Hymowitz et al., 2000). BR3 does not deviate from this approach in that contacts are made primarily from a single receptor loop, except that it manages to generate high-affinity BAFF-binding through interactions with one receptor domain. However, TACI_d2 binds APRIL using a continuous surface formed by residues from every secondary structural element in the domain. In so doing, the APRIL-TACI_d2 interface ends up being similar in overall size to the multi-domain TNFR binding sites (e.g., lymphotoxin-TNFR1 buries ˜2100 Å²), yet binds in a single site on the ligand analogous to BR3.

### Example 6

### Improving Ligand Binding in TACI_d1

A homology model of TACI_d1 was generated based on the structure of TACI_d2. TACI_d1 is predicted to adopt a similar DxL hairpin fold that could bind ligand in a similar fashion as that seen for the other APRIL/BAFF receptors. TACI_d1 is predicted to also share the same disulfide-connectivity and helical secondary structure for the C-terminal sub-module. However, the h1-h2 loop, that makes key contacts with the ligand in the APRIL-TACI_d2 complex, differs in length and amino acid sequence between TACI domains. Thus, these changes are likely to be responsible for the lower affinity of TACI_d1 for ligand binding.

Using this model of TACI_d1, a model of intact TACI_d1d2 was constructed. The connection between the two CRDs in TACI is different than in other multi-domain TNFR. Typically, there are only 1-2 residues between the last cysteine of a CRD and the first cysteine in the following CRD and these residues are part of a beta-strand. Instead in TACI, there are four residues between CRD1 and CRD2. This connection is unlikely to form a beta-strand as the final cysteine of CRD1 is expected to be part of a small helix, similar to that of TACT_d2 or BCMA. With uncertainty in the conformation of the connecting linker, the relative orientation of the two CRDs with respect to each other is difficult to predict. One could model CRD1 such that it touches the ligand surface, while CRD2 occupies the primary receptor-binding site. However, since the addition of CRD I adds no further binding energy compared to that of CRD2 alone, CRD1 does not likely make extensive contacts to ligand.

Despite the uncertainty in the orientation of TACI_d1 with respect to TACI_d2, some models of possible interactions between two-domain TACI with ligand can be ruled out on the basis of steric considerations. Docking the two domain construct of TACI to BAFF (pdb code 1JH5) (Liu et al., 2002) indicates that the hypothesis raised by Liu and coworkers (2003) that TACI might bridge two binding sites on adjacent trimers in the higher-order viral-like BAFF oligomer is physically impossible. In order for CRDs 1 and 2 to simultaneously bind DxL pockets on adjacent BAFF trimers, the final CRD1 cysteine (Cys66) would need to be ˜30 Å from the first cysteine (Cys71) in CRD2 which is farther than can be spanned by the four residue (⁶⁷RSLS⁷⁰) inter-domain linker. Similarly, TACI CRDs 1 and 2 cannot simultaneously bind in the same manner to two different APRIL (or BAFF) protomers in the same trimer as the distance between the C-terminal cysteine of CRD1 to the N-terminal cysteine of CRD2 would need to be ˜40 Å² to reach the two binding pockets.

### Example 7

### Mutational Analysis of TACI_d2

A combinatorial (“shotgun”) alanine scan (Weiss et al., 2000) of TACI_d2 was used to determine the contribution of individual amino acid side chains to the binding of either APRIL or BAFF. Briefly, three different libraries were generated to allow mutation of residues 72-109 (except positions where the wild-type residue is cysteine or alanine). Wild-type codons were replaced by degenerate codons, allowing residues to vary as the wild-type amino acid or alanine. For positions where the wild-type residue is Arg, Asn, Gln, H is, Ile, Leu, Phe, or Tyr, the shotgun code allows for two additional amino acid substitutions (Weiss et al., 2000). Similar analyses for BR3- and BCMA-binding to BAFF and/or APRIL were reported previously (Gordon et al., 2003; Patel et al., 2004).

(a) Phage Display of TACI_d2

First, an initial vector for phage display of the TACI_d2 extracellular domain was prepared by subcloning the fragment encoding residues 68-109 from the pET32a expression vector described above into the phagemid BCMA2-g3 described previously (Patel et al., 2004). The resulting construct (TACI_d2-g3) contained the N-terminal peptide epitope (MADPNRFRGKDLGG) (SEQ ID NO:78) for an antibody (3C8:2F4, Genentech, Inc.) followed by TACI_d2, an amber stop codon, and the C-terminal half of the M13 p3 coat protein. Expression was driven by the alkaline phosphatase promoter. TACI_d2-g3 was used to prepare the three “shotgun alanine” scanning libraries essentially as described previously (Weiss et al., 2000). A shotgun alanine codon coded for the wild-type residue, alanine, or one of two additional substitutions in certain cases, due to codon degeneracy, at a given position. Each of these libraries, prepared separately, contain shotgun codons at unique positions: library one has eleven shotgun codons at positions 72, 73, 74, 75, 76, 77, 78, 80, 81, 83, and 85, library two has thirteen shotgun codons at positions 79, 81, 82, 84, 87, 88, 91, 92, 94, 95, 96, 97, 98, and library three has eight shotgun codons at positions 99, 102, 103, 105, 106, 107, 108, and 109. Each library contained at least 1×10¹⁰ phage/ml, allowing for complete representation of the theoretical diversity (>10³-fold excess; libraries one, two, and three code for 2.6×10⁵, 4.2×10⁶, and 3.2×10⁴ unique sequences, respectively).

### (b) Shotgun Library Sorting and Analysis

Phage from each of the libraries described above were subjected to rounds of binding selection against APRIL, BAFF, or anti-tag antibody (3C8:2F4 Genentech, Inc.) immobilized on 96-well Nunc Maxisorp immunoplates (Sidhu, 2001). BSA-coated wells were used to determine non-specific background binding. Phage eluted from each target were propagated in E. coli XL1-Blue in the presence of M13K07 helper phage; amplified phage were used for selection against the same target in the previous round. Phage sorting was stopped when 100-fold enrichment was obtained (generally at rounds two or three). Enrichment was calculated from the ratio of the phage titer eluted from the target-coated wells to the phage titer eluted from the BSA-coated wells. Individual clones from each library and selection target were then grown in a 96-well format in 400 ul of 2YT medium supplemented with carbenicillin and KO7 helper phage. Phage ELISA assays (Weiss et al., 2000) were performed to detect phage-displayed variants of TACI_d2 capable of binding APRIL, BAFF, or anti-tag antibody. All clones tested that were found to be positive in their respective ELISAs were then sequenced as described previously (Sidhu, 2001). Sequences of acceptable quality were translated and aligned. For APRIL-binding, 71, 40, and 54 sequences were analyzed from libraries one, two and three, respectively. For BAFF-binding, 70, 50, and 53 sequences were analyzed for libraries one, two and three, respectively. For the display selection, a minimum of 47 sequences were analyzed for each library. The number of times a particular amino acid was found at each position was tabulated and the normalized wild-type/mutant functional ratio, F, was calculated for each position as described (Skelton et al., 2003) (Table 6). F values describe the effect of mutation on target binding, while accounting for differences in display efficiencies, with values >1 representing deleterious mutations and those <1 representing favorable mutations. Due to the relatively small number of sequences analyzed (see Experimental Procedures), only those mutations that showed a greater than 10-fold effect (i.e., F>10 or F<0.1) are considered significant.

A total of 12 TACI residues resulted in significant loss of affinity for APRIL and/or BAFF when mutated to alanine (F78, Y79, D80, L82, L83, I87, R84, 192, G94, Q95, H96 and P97) (Table 6, below). These residues map to a concave surface on the TACI_d2 structure and indicate that both sub-modules of TACI_d2 are important for ligand binding. Seven residues from the DxL hairpin showed significant effects, including both the “D” (Asp80) and “L” (Leu82) which are clearly essential for both APRIL and BAFF binding since the wild-type residue was always selected. Leu83 at the tip of the beta-turn was also relatively intolerant to substitution by alanine for either APRIL- or BAFF-binding, but was frequently substituted by valine, especially for binding BAFF (Table 6). Furthermore, a hydrophobic residue at position 87 is clearly important as only isoleucine (the wild-type residue) or valine was always selected for binding to both ligands. Residues from the C-terminal sub-module including those from h1 (Ile92) and the h1-h2 loop (residues 94-97) also showed contributions to ligand binding. In contrast, the h1-h2 loop of BCMA was not found to be important for ligand-binding (Patel et al., 2004); this loop is essentially absent in BR3 (Gordon et al., 2003). Gly94 and His96 might play a role in stabilizing the structure given that the glycine adopts a positive phi value (which would not be readily accommodated by alanine) and His96 is buried in the TACI_d2 structure, thus loss of binding upon alanine-substitution of these residues might be due to indirect effects. However, Gln95 and Pro97 line the concave surface of TACI_d2 and could contribute directly to ligand-binding (see below). Finally, several residues showed different effects on APRIL- and BAFF-binding and are likely to be involved in ligand specificity. For example, Phe78 was found to be important for APRIL binding (F value=29 for APRIL and 1.1 for BAFF), while mutation of Arg84 only showed losses on BAFF binding (F value=1.1 for APRIL and 16 for BAFF).

TABLE 6
Combinatorial Alanine Scan of TACI_d2

| | | BAFF | | | | APRIL | | | | Display | | | | | | | | | |
|---|---|---|---|---|---|---|---|---|---|---|---|---|---|---|---|---|---|---|---|
| | | selection | | | | selection | | | | selection | | | | F (BAFF) | | | F (APRIL) | | |
| residue | m2, m3 | wt | Ala | m2 | m3 | wt | Ala | m2 | m3 | wt | Ala | m2 | m3 | Ala | m2 | m3 | Ala | m2 | m3 |
| R72 | G, P | 14 | 20 | 24 | 12 | 21 | 10 | 25 | 15 | 18 | 9 | 12 | 9 | 0.4 | 0.4 | 0.6 | 1.1 | 0.6 | 0.7 |
| K73 | E, T | 23 | 20 | 18 | 9 | 20 | 22 | 15 | 14 | 4 | 7 | 23 | 14 | 2.0 | 7.3 | 8.9 | 1.6 | 7.7 | 5.0 |
| E74 | | 38 | 31 | | | 41 | 30 | | | 32 | 16 | | | 0.6 | | | 0.7 | | |
| Q75 | E, P | 17 | 14 | 21 | 18 | 20 | 10 | 25 | 16 | 14 | 10 | 12 | 12 | 0.9 | 0.7 | 0.8 | 1.4 | 0.7 | 1.1 |
| G76 | | 42 | 28 | | | 46 | 25 | | | 26 | 22 | | | 1.3 | | | 1.6 | | |
| K77 | E, T | 19 | 12 | 21 | 18 | 19 | 21 | 14 | 17 | 13 | 14 | 13 | 8 | 1.7 | 0.9 | 0.6 | 1.0 | 1.4 | 0.7 |
| F78 | S, V | 29 | 12 | 6 | 23 | 62 | 1 | 0 | 8 | 15 | 7 | 16 | 10 | 1.1 | 5.2 | 0.8 | 29 | >66 | 5.2 |
| Y79 | D, S | 45 | 4 | 0 | 0 | 39 | 0 | 0 | 1 | 19 | 10 | 4 | 15 | 5.9 | >9.5 | >36 | >21 | >8.2 | 31 |
| D80 | | 70 | 0 | | | 71 | 0 | | | 30 | 18 | | | >42 | | | >43 | | |
| H81 | D, P | 25 | 7 | 8 | 49 | 23 | 6 | 11 | 24 | 19 | 10 | 8 | 30 | 1.9 | 1.3 | 0.8 | 2.0 | 0.9 | 1.5 |
| L82 | P, V | 50 | 0 | 0 | 0 | 40 | 0 | 0 | 0 | 14 | 10 | 17 | 7 | >36 | >61 | >25 | >29 | >49 | >20 |
| L83 | P, V | 41 | 3 | 1 | 25 | 57 | 2 | 0 | 12 | 14 | 15 | 5 | 14 | 15 | 15 | 1.6 | 31 | >20 | 4.8 |
| R84 | G, P | 45 | 2 | 3 | 0 | 19 | 12 | 9 | 0 | 13 | 9 | 11 | 15 | 16 | 13 | >52 | 1.1 | 1.8 | >22 |
| D85 | | 32 | 37 | | | 26 | 45 | | | 25 | 23 | | | 0.8 | | | 0.5 | | |
| C86 | | | | | | | | | | | | | | | | | | | |
| I87 | T, V | 28 | 0 | 0 | 22 | 25 | 0 | 0 | 15 | 13 | 14 | 10 | 11 | >30 | >22 | 1.1 | >27 | >19 | 1.4 |
| S88 | | 35 | 15 | | | 32 | 8 | | | 24 | 23 | | | 2.2 | | | 3.8 | | |
| C89 | | | | | | | | | | | | | | | | | | | |
| A90 | | | | | | | | | | | | | | | | | | | |
| S91 | | 33 | 17 | | | 30 | 10 | | | 28 | 20 | | | 1.4 | | | 2.1 | | |
| I92 | T, V | 12 | 3 | 9 | 26 | 26 | 2 | 3 | 9 | 13 | 11 | 9 | 15 | 3.4 | 0.9 | 0.5 | 11 | 6.0 | 3.3 |
| C93 | | | | | | | | | | | | | | | | | | | |
| G94 | | 46 | 4 | | | 39 | 1 | | | 25 | 22 | | | 10 | | | 34 | | |
| Q95 | E, P | 46 | 2 | 1 | 0 | 33 | 5 | 2 | 0 | 11 | 11 | 13 | 13 | 23 | 58 | >54 | 6.6 | 21 | >39 |
| H96 | D, P | 44 | 3 | 0 | 3 | 36 | 3 | 0 | 1 | 12 | 12 | 8 | 16 | 15 | >29 | 20 | 12 | >24 | 48 |
| P97 | | 47 | 3 | | | 40 | 0 | | | 32 | 16 | | | 7.8 | | | >20 | | |
| K98 | E, T | 31 | 13 | 1 | 5 | 23 | 8 | 4 | 5 | 10 | 12 | 12 | 14 | 2.9 | 37 | 8.7 | 3.5 | 6.9 | 6.4 |
| Q99 | E, P | 14 | 9 | 17 | 13 | 23 | 8 | 8 | 15 | 14 | 15 | 13 | 23 | 1.7 | 0.8 | 1.8 | 1.9 | 1.6 | 1.5 |
| C100 | | | | | | | | | | | | | | | | | | | |
| A101 | | | | | | | | | | | | | | | | | | | |
| Y102 | D, S | 9 | 7 | 12 | 25 | 12 | 9 | 9 | 24 | 9 | 14 | 7 | 35 | 2.0 | 0.6 | 1.4 | 1.6 | 0.8 | 1.5 |
| F103 | S, V | 23 | 7 | 8 | 15 | 26 | 7 | 4 | 17 | 23 | 9 | 23 | 10 | 1.3 | 2.9 | 0.7 | 1.3 | 5.8 | 0.6 |
| C104 | | | | | | | | | | | | | | | | | | | |
| E105 | | 30 | 23 | | | 29 | 25 | | | 22 | 43 | | | 1.9 | | | 1.7 | | |
| N106 | D, T | 6 | 23 | 12 | 12 | 13 | 17 | 15 | 9 | 12 | 21 | 18 | 14 | 0.9 | 1.5 | 1.2 | 1.2 | 1.2 | 1.6 |
| K107 | E, T | 10 | 20 | 8 | 15 | 8 | 24 | 7 | 14 | 12 | 22 | 15 | 16 | 1.1 | 1.9 | 1.1 | 0.9 | 2.1 | 1.1 |
| L108 | P, V | 15 | 9 | 17 | 12 | 10 | 11 | 18 | 15 | 15 | 11 | 25 | 14 | 1.2 | 1.5 | 1.2 | 1.0 | 1.4 | 0.9 |
| R109 | G, P | 8 | 10 | 16 | 19 | 12 | 6 | 16 | 20 | 14 | 9 | 19 | 23 | 0.9 | 1.2 | 1.2 | 1.5 | 1.2 | 1.2 |

The occurrence of the wild-type residue (wt) or each mutation (Ala, m2, m3) found among sequenced clones following two rounds of binding by ligand selection (BAFF or APRIL) or display selection (anti-tag) is shown for the scanned positions in TACI_d2. The occurrence of wild-type divided by mutant provides a wt/mutant ratio for each position (not shown). A normalized frequency ratio (F) was calculated to quantify the effect of each mutation on ligand-binding while accounting for display efficiencies: i.e. F=[wt/mutant (ligand selection)]÷[(wt/mutant (display selection)]. Deleterious mutations have ratios >1, while advantageous mutations have ratios <1; boldface values indicate a >10-fold effect and are considered significant. Certain F values represent a lower limit since Ala, m2, or m3 were not observed at these sites in ligand selection. Generally, any F value that was approximately 10 or less was considered to indicate that the particular residue change was somewhat tolerated for binding and that a value of 5 or less was more tolerant for binding.

### Example 8

### Phage Optimization Studies

NNS library construction and sorting. Mutagenesis results for residues Phe78, Tyr79, Arg84, and Ile92 suggest these positions are likely candidates for providing ligand specificity since Ala substitution had different effects on BAFF and APRIL binding. Positions Phe78, Tyr79, Arg84, and Ile92 were selected for further phage optimization studies by incorporation of NNS degenerate codons at these positions in the TACI CRD2-g3 phagemid followed by selection for ligand binding (NNS degenerate codon as defined by IUB code (Sidhu, S. S., et al. (2000) Methods Enzymol 328, 333-363). The library contained 3×10⁸ phage/ml allowing complete representation of the library, theoretically 1.6×10⁵ unique members.

This new phage library was subjected to sorting against either BAFF, APRIL or the display target antibody. After 3 rounds of sorting, approximately 48 clones were picked from each selection and the DNA sequences of the phage clones were determined. Since each amino acid position selected for NNS codon introduction has the potential of all 20 amino acids from 31 triplet codons, the data was weighted according to codon degeneracy by calculating the ratio of percent occurrence to percent degeneracy of the amino acid at a given position as suggested previously (LaBean, T. H., and Kauffman, S. A. (1993) Protein Sci 2, 1249-1254). The normalized F′ value corrects for display bias and is calculated as the percent occurrence to percent degeneracy ratio for ligand selection divided by the percent occurrence to percent degeneracy ratio for display efficiency. Percent occurrence is calculated by dividing the number of times a particular amino acid appeared at a particular position by the total number of amino acids sequenced at that position followed by multiplying by 100. Percent degeneracy is calculated by dividing the degeneracy in code for a particular amino acid divided by the total degeneracy possible at that position followed by multiplying by 100 (e.g., if only A, G and I were selected, total degeneracy possible would be 5). A large F′ value for an amino acid at a given position indicates that the amino acid is a favorable substitution for binding to the target ligand.

As shown in Table 7, the substitutions that result in the maximum difference in F′ value between APRIL selection and BAFF selection are F78E (favors BAFF), Y79E (favors APRIL), R84D, R84E or R84W (favors APRIL), and I92L (favors APRIL). Any value over 0 indicates that the mutant containing the residue change was selected and therefore the residue was somewhat tolerated for binding.

TABLE 7

| | F′ BAFF | | | | F′ APRIL | | | |
|---|---|---|---|---|---|---|---|---|
| a.a. | F78 | Y79 | R84 | I92 | F78 | Y79 | R84 | I92 |
| A | 0 | 0 | 3 | 0 | 0 | 0 | 2 | 0 |
| C | 0 | 0 | 0 | 0 | 0 | 0 | 0 | 0 |
| D | 0 | 0 | 0 | 0 | 0 | 0 | 9 | 0 |
| E | 5 | 0 | 0 | 0 | 0 | 8 | 3 | 0 |
| F | 3 | 5 | 7 | 0 | 2 | 4 | 9 | 0 |
| G | 0 | 0 | 0 | 0 | 0 | 0 | 1 | 0 |
| H | 0 | 0 | 1 | 0 | 0 | 0 | 0 | 0 |
| I | 10 | 6 | 0 | 1 | 7 | 4 | 0 | 2 |
| K | 0 | 0 | 1 | 0 | 0 | 0 | 0 | 0 |
| L | 0 | 1 | 2 | 0 | 0 | 0 | 3 | 2 |
| M | 1 | 0 | 7 | 0 | 0 | 0 | 18 | 0 |
| N | 0 | 0 | 1 | 0 | 0 | 0 | 0 | 0 |
| P | 0 | 3 | 0 | 0 | 0 | 2 | 0 | 0 |
| Q | 0 | 0 | 0 | 0 | 0 | 0 | 0 | 0 |
| R | 0 | 0 | 5 | 0 | 0 | 0 | 0 | 0 |
| S | 0 | 0 | 0 | 0 | 0 | 0 | 0 | 0 |
| T | 0 | 0 | 3 | 7 | 1 | 0 | 0 | 3 |
| V | 1 | 2 | 2 | 9 | 1 | 0 | 4 | 6 |
| W | 0 | 4 | 0 | 0 | 0 | 3 | 9 | 0 |
| Y | 41 | 2 | 1 | 0 | 61 | 4 | 0 | 0 |

Based on the results described in Table 7 and Table 6, Table 8 lists some of the residues that are tolerated at each indicated position. Bolded lettering indicates naturally occurring residues in human TACI, and bolded and italicized lettering indicates exemplary residues that can increase the specificity of a polypeptide for April or BAFF compared to a wild-type TACI sequence.

TABLE 8

| Residue | | | |
|---|---|---|---|
| Numbering | | Examples of | Examples of Tolerated |
| In Native | Residue | Tolerated Residues | Residues |
| human TACI | No. | For BAFF Binding | For APRIL Binding |
| 71 | 1 | C | C |
| 72 | 2 | R, A, G, P | R, A, G, P |
| 73 | 3 | K, A, E, T | K, A, E, T |
| 74 | 4 | E, A | E, A |
| 75 | 5 | Q, A, E, P | Q, A, E, P |
| 76 | 6 | G, A | G, A |
| 77 | 7 | K, A, E, T | K, A, E, T |
| 78 | 8 | F, A, V, I, M, [symbol] , [symbol] | F, V, T, I, Y |
| 79 | 9 | Y, A, F, W, L, I, P, [symbol] | Y, I, P, F, W, [symbol] |
| 80 | 10 | D | D |
| 81 | 11 | H, A, D, P | H, A, D, P |
| 82 | 12 | L | L |
| 83 | 13 | L, V | L, V |
| 84 | 14 | R, L, A, K, F, H, | R, A, G, L, V, [symbol] , [symbol] , |
| | | M, N, T, Y | [symbol] , [symbol] , [symbol] |
| 85 | 15 | D, A | D, A |
| 86 | 16 | C | C |
| 87 | 17 | I, V | I, V |
| 88 | 18 | S, A | S, A |
| 89 | 19 | C | C |
| 90 | 20 | A | A |
| 91 | 21 | S, A | S, A |
| 92 | 22 | I, V, T, A | I, V, T, [symbol] |
| 93 | 23 | C | C |
| 94 | 24 | G | G |
| 95 | 25 | Q | Q, A |
| 96 | 26 | H | H |
| 97 | 27 | P, A | P |
| 98 | 28 | K, A, T | K, A, E, T |
| 99 | 29 | Q, A, E, P | Q, A, E, P |
| 100 | 30 | C | C |
| 101 | 31 | A | A |
| 102 | 32 | Y, A, D, S | Y, A, D, S |
| 103 | 33 | F, A, V, S | F, A, V, S |
| 104 | 34 | C | C |
| 105 | 35 | E, A | E, A |
| 106 | 36 | N, A, D, T | N, A, D, T |
| 107 | 37 | K, A, E, T | K, A, E, T |
| 108 | 38 | L, A, P, V | L, A, P, V |
| 109 | 39 | R, A, G, P | R, A, G, P |

### Example 9

### TACI CRD1 Loop Swap

A PCR product containing amino acids R32-R67 from CRD1 of human TACI was cloned into a modified pET-32a vector with a deleted S-Tag and enterokinase site. A two-step PCR approach was used to substitute residues NHQSQRT of hTACI CRD1 with residues GQHPKQ of hTACI CRD2. pET-32a-hTACI CRD1 LS was expressed in Origami (DE3) competent cells (Novagen) following IPTG induction overnight at 16° C.

hTACI CRD1 LS cell pellets were lysed in buffer A (20 mM CAPS pH 9.7, 400 mM NaCl, 2 mM PMSF, 0.2 mM benzamidine, and 5 mM imidazole) using microfluidization. Cell supernatant was eluted from a Ni-NTA agarose column (Quiagen) in buffer A containing 50 mM imidazole. Protein was passed over a S75 sizing column in buffer B (20 mM Tris pH 8.2, 400 mM NaCl). The thioredoxin-His₆ tag was removed by thrombin digestion at 4° C. overnight. Untagged hTACI CRD1 LS was purified on a S75 sizing column in buffer C (PBS with 150 mM NaCl pH 7.0).

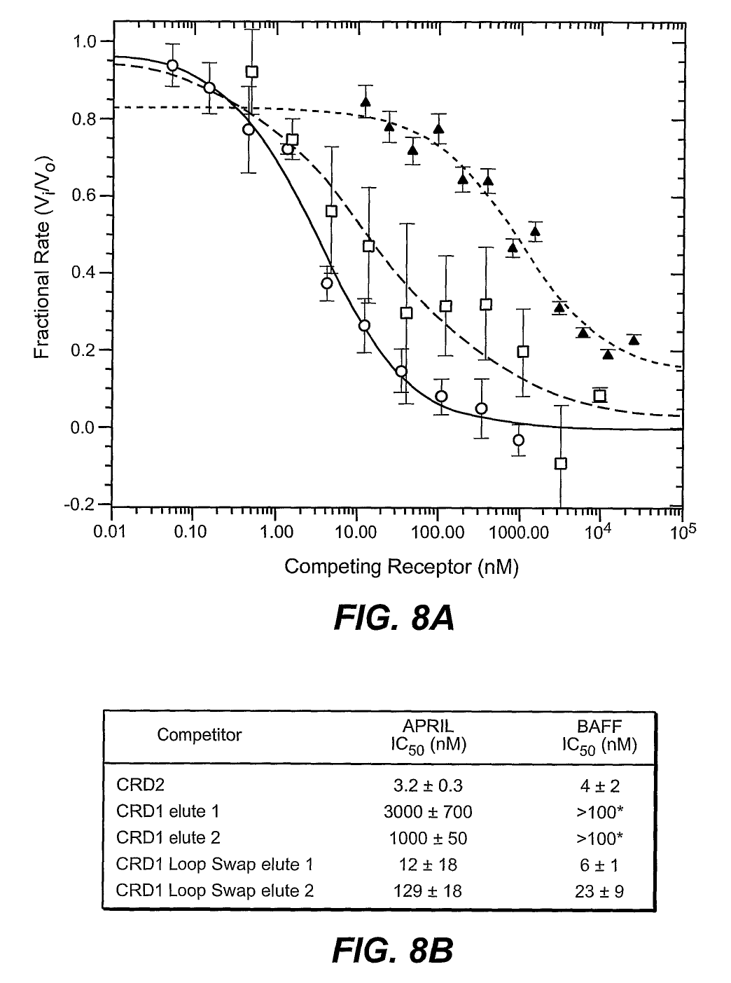

Competitive surface plasmon resonance experiments to measure binding to APRIL or BAFF were performed as described in Example 1. FIG. 8A shows the competitive inhibition of APRIL-binding to BCMA-Fc by TACI variants: TACI CRD2 (open circle), TACI CRD1 Loop Swap elute 1 (open square) and TACI CRD1 elute I (filled triangle). FIG. 8B shows the IC50 values for the competitive binding to APRIL and BAFF, as calculated from the mean of two (TACI CRD1) or three (TACI CRD2, TACI_CRD1 Loop Swap) independent experiments). The substitution of residues from TACI CRD2 into TACI CRD1 increased TACI CRD1 affinity for APRIL and BAFF.

### REFERENCES

The following publications are incorporated by reference herein.
Banner, D. W., D'Arcy, A., Janes, W., Gentz, R., Schoenfeld, H.-J., Broger, C., Loetscher, H., and Lesslauer, W. (1993). Crystal Structure of the Soluble Human 55 kd TNF Receptor-Human TNFb Complex: Implications for TNF Receptor Activation. Cell 73, 431-445.
Bodmer, J.-L., Schneider, P., and Tschopp, J. (2002). The Molecular Architecture of the TNF Superfamily. Trends Biochem Sci 27, 19-26.
Cavanagh, J., Fairbrother, W. J., Palmer, A. G., III, and Skelton, N.J. (1995). Protein NMR Spectroscopy: Principles and Practice (San Diego, Academic Press). CCP4 (1994). The CCP4 suite: programs for protein crystallography. Acta Cryst D 50, 760-763.
Cha, S.-S., Sung, B.-J., Kim, Y.-A., Song, Y.-L., Kim, H.-J., Kim, S., Lee, M.-S., and Oh, B.-H. (2000). Crystal Structure of TRAIL-DR5Complex Identifies a Critical Role of the Unique Frame Insertion in Conferring Recognition Specificity. J Biol Chem 275, 31171-31177.
Chan, F. K.-M., Chun, H. J., Zheng, L., Siegel, R. M., Bui, K. L., and Lenardo, M. J. (2000). A domain in TNF receptors that mediates ligand-independent receptor assembly and signaling. Science 288, 2351-2354.
Cochran, A. G., Tong, R. Y., Starovasnik, M. A., Park, E. J., McDowell, R. S., Theaker, J. E., and Skelton, N. J. (2001). A Minimal Peptide Scaffold for b-Turn Display: Optimizing a Strand Position in Disulfide Cyclized b-Hairpins. J Am Chem Soc 123, 625-632.
Cornilescu, G., Delaglio, F., and Bax, A. (1999). Protein Backbone Angle Restraints from Searching a Database for Chemical Shift and Sequence Homology. J Biomol NMR 13, 289-302.
Gordon, N. C., Pan, B., Hymowitz, S. G., Yin, J., Kelley, R. F., Cochran, A. G., Yan, M., Dixit, V. M., Fairbrother, W. J., and Starovasnik, M. A. (2003). BAFF/BLyS Receptor 3 comprises a minimal TNF receptor-like module that encodes a highly focused ligand-binding site. Biochemistry 42, 5977-5983.
Hahne, M., Kataoka, T., Schröter, M., Hofmann, K., Irmler, M., Bodmer, J.-L., Schneider, P., Bornand, T., Holler, N., French, L. E., et al. (1998). APRIL, a new ligand of the tumor necrosis factor family stimulates tumor cell growth. J Exp Med 188, 1185-1190.
Herrmann, T., Güntert, P., and Wüthrich, K. (2002). Protein NMR structure determination with automated NOE assignment using the new software CANDID and the torsion angle dynamics algorithm DYANA. J Mol Biol 319, 209-227.
Hymowitz, S. G., Christinger, H. W., Fuh, G., Ultsch, M., O'Connell, M., Kelley, R. F., Ashkenazi, A., and de Vos, A. M. (1999). Triggering Cell Death: The Crystal Structure of Apo2L/TRAIL in a Complex with Death Receptor 5. Mol Cell 4, 563-571.
Hymowitz, S. G., O'Connell, M. P., Ultsch, M. H., Hurst, A., Totpal, K., Ashkenazi, A., de Vos, A. M., and Kelley, R. F. (2000). A unique zinc-binding site revealed by a high-resolution X-ray structure of homotrimeric Apo2L/TRAIL. Biochemistry 39, 633-640.
Karpusas, M., Cachero, T. G., Qian, F., Boriack-Sjodin, A., Mullen, C., Strauch, K., Hsu, Y.-M., and Kalled, S. L. (2002). Crystal Structure of Extracellular Human BAFF, a TNF Family Member that Stimulates B Lymphocytes. J Mol Biol 315, 1145-1154.

Kayagaki, N., Yan, M., Seshasayee, D., Wang, H., Lee, W., French, D. M., Grewal, I. S., Cochran, A. G., Gordon, N. C., Yin, J., et al. (2002). BAFF/BLyS Receptor 3 Binds the B Cell Survival Factor BAFF Ligand through a Discrete Surface Loop and Promotes Processing of NF-kB2. Immunity 17, 515-524.
Kim, H. M., Yu, K. S., Lee, M. E., Shin, D. R., Kim, Y. S., Paik, S.-G., Yoo, O. J., Lee, H., and Lee, J.-O. (2003). Crystal structure of the BAFF-BAFF-R complex and its implications for receptor activation. Nat Struct Biol 10, 342-348.
Liu, Y., Hong, X., Kappler, J., Jiang, L., Zhang, R., Xu, L., Pan, C.-H., Martin, W. E., Murphy, R. C., Shu, H.-B., et al. (2003). Ligand-receptor binding revealed by the TNF family member TALL-1. Nature 423, 49-56.
Liu, Y., Xu, L., Opalka, N., Kappler, J., Shu, H.-B., and Zhang, G. (2002). Crystal Structure of sTALL-1 Reveals a Virus-Like Assembly of TNF Family Ligands. Cell 108, 383-394.
Locksley, R. M., Killeen, N., and Lenardo, M. J. (2001). The TNF and TNF receptor superfamilies: integrating mammalian biology. Cell 104, 487-501.
Mackay, F., Schneider, P., Rennert, P., and Browning, J. (2003). BAFF and APRIL: A tutorial on B cell survival. Annu Rev Immunol 21, 231-264.
Marsters, S. A., Yan, M., Pitti, R. M., Haas, P., Dixit, V. M., and Ashkenazi, A. (2001). Interaction of the TNF homologues BLyS and APRIL with the TNF receptor homologues BCMA and TACI. Curr Biol 10, 785-788. Medema, J. P., Planelles-Carazo, L., G., H., and Hahne, M. (2003). The uncertain glory of APRIL. Cell Death and Differentiation 10, 1121-1125.
Mongkolsapaya, J., Grimes, J. M., Chen, N., Xu, X.-N., Stuart, D. I., Jones, E. Y., and Screaton, G. R. (1999). Structure of the TRAIL-DR5Complex Reveals Mechanisms Conferring Specificity in Apoptotic Initiation. Nat. Struct Biol 6, 1048-1053.
Moore, P. A., Belvedere, O., Orr, A., Pieri, K., LaFleur, d. W., Feng, P., Soppet, D., Charters, M., Gentz, R., Parmelee, D., et al. (1999). BLyS: member of the tumor necrosis factor family and B lymphocyte stimulator. Science 285, 260-263.
Neri, D., Szyperski, T., Otting, G., Senn, H., and Wuthrich, K. (1989). Stereospecific nuclear magnetic resonance assignments of the methyl groups of valine and leucine in the DNA-binding domain of the 434 repressor by biosynthetically directed fractional ¹³C labeling. Biochemistry 28, 7510-7516.
O'Connor, B. P., Raman, V. S., Erickson, L. D., Cook, W. J., Weaver, L. K., Ahonen, C., Lin, L.-L., Mantchev, G. T., Bram, R. J., and Noelle, R. J. (2004). BCMA is essential for the survival of long-lived bone marrow plasma cells. J Exp Med 199, 91-97.
Oren, D. A., Li, Y., Volovik, Y., Morris, T. S., Dharia, C., Das, K., Galperina, O., Gentz, R., and Arnold, E. (2002). Structural Basis of BLyS Receptor Recognition. Nat. Struct Biol 9, 288-292.
Otwinowski, Z., and Minor, W. (1997). Processing of X-ray diffraction data collected in oscillation mode. Meth Enzymol 176, 307-326.
Patel, D. R., Wallweber, H. J. A., Yin, J., Shriver, S. K., Marsters, S. A., Gordon, N. C., Starovasnik, M. A., and Kelley, R. F. (2004). Engineering an APRIL-specific B Cell Maturation Antigen. J Biol Chem 279, 16727-16735.
Pelletier, M., Thompson, J. S., Qian, f., Bixler, S. A., Gong, D., Cachero, T., Gilbride, K., Day, E., Zafari, M., Benjamin, C., et al. (2003). Comparison of soluble decoy IgG fusion proteins of BAFF-R and BCMA as antagonists for BAFF. J Biol Chem 278, 33127-33133.
Rennert, P., Schneider, P., Cachero, T. G., Thompson, J., Trabach, L., Hertig, S., Holler, N., Qian, F., Mullen, C., Strauch, K., et al. (2000). A soluble form of B Cell Maturation Antigen, a receptor for the tumor necrosis factor family member APRIL, inhibits tumor cell growth. J Exp Med 192, 1677-1684.
Schiemann, B., Gommerman, J. L., Vora, K., Cachero, T. G., Shulga-Morskaya, S., Dobles, M., Frew, E., and Scott, M. L. (2001). An Essential Role for BAFF in the Normal Development of B Cells Through a BCMA-Independent Pathway. Science 293, 2111-2114.
Schneider, P., MacKay, F., Steiner, V., Hofmann, K., Bodmer, J.-L., Holler, N., Ambrose, C., Lawton, P., Bixler, S., Acha-Orbea, H., et al. (1999). BAFF, a novel ligand of the tumor necrosis factor family stimulates B cell growth. J Exp Med 189, 1747-1756.
Seshasayee, D., Valdez, P., Yan, M., Dixit, V. M., Tumas, D., and Grewal, I. S. (2003). Loss of TACI causes fatal lymphoproliferation and autoimmunity, establishing TACI as an inhibitory BLyS receptor. Immunity 18, 279-288.
Sidhu, S. S. (2001). Engineering M13 for phage display. Biomol Eng 18, 57-63.
Skelton, N. J., Akke, M., Kordel, J., Palmer, A. G. P., Rance, M., and Chazin, W. J. (1993). Practical aspects of the measurement of heteronuclear relaxation parameters. J Magn Reson B 102, 253-264.
Skelton, N. J., Koehler, M. F. T., Zobel, K., Wong, W. L., Yeh, S., Pisabarro, M. T., Yin, J., Lasky, L. A., and Sidhu, S. S. (2003). Origins of PDZ domain ligand specificity. J Biol Chem 278, 7645-7654.
Thompson, J. S., Bixler, S. A., Qian, F., Vora, K., Scott, M. L., Cachero, T. G., Hession, C., Schneider, P., Sizing, I. D., Mullen, C., et al. (2001). BAFF-R, A newly identified TNF receptor that specifically interacts with BAFF. Science 293, 2108-2111.
Thompson, J. S., Schneider, P., Kalled, S. L., Wang, L., Lefevre, E. A., Cachero, T. G., MacKay, F., Bixler, S. A., Zafari, M., Liu, Z.-Y., et al. (2000). BAFF binds to the Tumor Necrosis Factor receptor-like molecule B Cell Maturation Antigen and is important for maintaining the peripheral B cell population. J Exp Med 192, 129-135.
von Bülow, G.-U., and Bram, R. J. (1997). NF-AT Activation induced by a CAML-interacting member of the tumor necrosis factor receptor family. Science 278, 138-141.
Wallweber, H. J. A., Compaan, D. M., Starovasnik, M. A., and Hymowitz, S. G. (2004). The crystal structure of A PRoliferation-Inducing Ligand, APRIL. J Mol Biol in press.
Weiss, G. A., Watanabe, C. K., Zhong, A., Goddard, A., and Sidhu, S. S. (2000). Rapid mapping of protein functional epitopes by combinatorial alanine scanning. Proc Natl Acad Sci USA 97, 8950-8954.
Wu, Y., Bressette, D., Carrell, J. A., Kaufman, T., Feng, P., Taylor, K., Gan, Y., Cho, Y. H., Garcia, A. D., Gollatz, e., et al. (2000). Tumor Necrosis Factor (TNF) receptor superfamily member TACI is a high affinity receptor for TNF family members APRIL and BLyS. J Biol Chem 275, 35478-35485.
Yan, M., Brady, J. R., Chan, B., Lee, W. P., Hsu, B., Harless, S., Cancro, M., Grewal, I. S., and Dixit, V. M. (2001a). Identification of a novel Receptor for B Lymphocyte Stimulator that is mutated in a mouse strain with severe B cell deficiency. Curr Biol 11, 1547-1552.
Yan, M., Marsters, S. A., Grewal, I. S., Wang, H., Ashkenazi, A., and Dixit, V. M. (2000). Identification of a Receptor for BLyS Demonstrates a Crucial Role in Humoral Immunity. Nat. Immunol 1, 37-41.
Yan, M., Wang, H., Chan, B., Roose-Girma, M., Erickson, S., Baker, T., Tumas, D., Grewal, I. S., and Dixit, V. M. (2001b). Activation and Accumulation of B Cells in TACI-deficient Mice. Nat. Immunol 2, 638-643. Zhou, G., Ke, R., Li, H., Zheng, G., Shen, C., Lin, L., and Yang, S. (2003). Transmembrane activator and CAML interactor (homo sapiens). NCBI/Genbank #AAP57629.

### Other Embodiments

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Accession numbers, as used herein, refer to Accession numbers from multiple databases, including GenBank, the European Molecular Biology Laboratory (EMBL), the DNA Database of Japan (DDBJ), or the Genome Sequence Data Base (GSDB), for nucleotide sequences, and including the Protein Information Resource (PIR), SWISSPROT, Protein Research Foundation (PRF), and Protein Data Bank (PDB) (sequences from solved structures), as well as from translations from annotated coding regions from nucleotide sequences in GenBank, EMBL, DDBJ, or RefSeq, for polypeptide sequences. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. Also incorporated by reference in their entirety herein are U.S. Provisional Application Nos. 60/625,341, filed Nov. 4, 2004 and 60/673,127, filed Apr. 19, 2005. The invention includes all embodiments and variations substantially as hereinbefore described and with reference to the examples and drawings.

1. A polypeptide comprising a CRD sequence, wherein the CRD sequence comprises at least the following: six cysteine residues, a D-Xa-L motif between the first and second cysteine residues and residues Xb-Q-H-Xc (SEQ ID NO:72) immediately C-terminal to the fourth cysteine residue, wherein Xa is any amino acid residue except C, Xb is G, T, or N and Xc is P, L or M, wherein the CRD sequence is not a CRD sequence of a naturally occurring TACI polypeptide.

2. A polypeptide comprising a CRD sequence, wherein the CRD sequence comprises at least the following: six cysteine residues, a D-Xa-L motif between the first and second cysteine residues and residues G-Xg-Xh-P (SEQ ID NO:73) immediately C-terminal to the fourth cysteine residue,
wherein Xa is any amino acid residue except C;
wherein Xg is any amino acid residue except C, E or P;
wherein Xh is any amino acid except C, A, D or P, and
wherein the CRD sequence is not a CRD sequence of a naturally occurring TACI polypeptide.

3. The polypeptide according to claim 2, wherein the CRD sequence is the sequence of Formula I:
C-X2-X3-X4-X5-X6-X7-X8-X9-D-X11-L-X13-X14-X15-C-X17-X18-C-X20-X21-X22-C-G-X25-X26-P—X28-X29-X30-C-X32-X33-X34-C (SEQ ID NO:1)
wherein X2-X3, X6-X9, X11, X13-X15, X17-X18, X20-X22 and X32-X34 are any amino acid except C.
wherein X4 is any amino acid except C or is absent;
wherein X5 is any amino acid except C or is absent;
wherein X25 is any amino acid residue except C, E or P;
wherein X26 is any amino acid except C, A, D or P;
wherein X28 is K, Q, A, R, N, H or S;
wherein X29 is any amino acid except C;
wherein X30 is any amino acid except C or is absent; and
wherein Formula I is not SEQ ID NO:4, SEQ ID NO:6 or SEQ ID NO:70.

4. The polypeptide according to claim 1, wherein the CRD sequence is the sequence of Formula II:
C-X2-X3-X4-X5-X6-X7-X8-X9-D-X11-L-X13-X14-X15-C-X17-X18-C-X20-X21-X22-C-X24-Q-H—X27-X28-X29-X30-C-X32-X33-X34-C (SEQ ID NO:2)
wherein X2-X3, X6-X9, X11, X13-X15, X17-X18, X20-X22 and X32-X34 are any amino acid except C;
wherein X4 is any amino acid except C or is absent;
wherein X5 is any amino acid except C or is absent;
wherein X24 is G, T, or N;
wherein X27 is P, L or M;
wherein X28 is K, Q, A, R, N, H or S;
wherein X29 is any amino acid except C;
wherein X30 is any amino acid except C or is absent; and
wherein Formula II is not SEQ ID NO:4, SEQ ID NO:6 or SEQ ID NO:70.

5. A polypeptide comprising an altered CRD1 sequence of a TACI polypeptide, wherein the altered CRD1 sequence comprises at least the following: six cysteine residues, a D-Xa-L motif between the first and second cysteine residues and residues Xb-Q-H-Xc (SEQ ID NO:72) immediately C-terminal to the fourth cysteine residue, wherein Xa is any amino acid residue except C, Xb is G, T, or N and Xc is P, L or M, wherein the CRD sequence is not a CRD sequence of a naturally-occurring TACI polypeptide.

6. The polypeptide according to claim 1, wherein the amino acid sequence between the fourth and fifth cysteine residues of the CRD sequence is selected from the group consisting of Xb-Q-H-Xc-Xd-Xe (SEQ ID NO:76) and Xb-Q-H-Xc-Xd-Xe-Xf (SEQ ID NO:77),
wherein Xb is G, T, or N;
wherein Xc is P, L or M;
wherein Xd is K, Q, A, R, N, H or S;
wherein Xe is any amino acid except C; and
wherein Xf is any amino acid except C, or is absent.

7. The polypeptide according to claim 1, wherein the CRD sequence is Formula III:
C-X2-X3-X4-X5-X6-X7-D-X9-L-X11-X12-X13-C-X15-X16-C-X18-X19-X20-C-X22-Q-H—X25-X26-X27-X28-C-X30-X31-X32-C (SEQ ID NO: 3),
wherein X2-X7, X9, X11-X13, X15-X16, X18-X20 and X30-X32 are any amino acid except C.
wherein X22 is G, T, or N;
wherein X25 is P, L or M;
wherein X26 is K, Q, A, R, N, H or S;
wherein X27 is any amino acid except C;
wherein X28 is any amino acid except C or is absent; and
wherein Formula III is not SEQ ID NO:8 or SEQ ID NO:9.

8-14. (canceled)

15. The polypeptide according to claim 1 or 2, wherein the polypeptide has increased specificity for BAFF over APRIL as compared to a naturally occurring TACI CRD2 sequence.

16. The polypeptide according to claim 15, wherein the polypeptide is characterized by one or more of the following: (a) the second residue N-terminal to the D-Xa-L motif is not F or Y; (b) the second residue N-terminal to the D-Xa-L motif is E or S; (c) the first residue N-terminal to the D-Xa-L motif is not Y or W; (d) the first residue N-terminal to the D-Xa-L motif is V; and (e) the second residue N-terminal to the D-Xa-L motif is E or S and the first residue N-terminal to the D-Xa-L motif is V.

17-20. (canceled)

21. The polypeptide according to claim 15, wherein the polypeptide binds BAFF with an IC50 value of 500 nM or less, 100 nM or less, 50 nM or less, 10 nM or less, 5 nM or less or mM or less.

22. The polypeptide according to claim 1 or 2, wherein the polypeptide has increased specificity for APRIL over BAFF as compared to a naturally occurring TACI CRD2 sequence.

23. The polypeptide according to claim 22, wherein the polypeptide is characterized by one or more of the following: (a) the first residue N-terminal to the D-Xa-L motif is not Y or W; (b) the first residue N-terminal to the D-Xa-L motif is E; (c) the second residue C-terminal to the D-Xa-L motif is not R or G or K; (d) the second residue C-terminal to the D-Xa-L motif is selected from the group consisting of E, D, W, F and M; (e) the first residue N-terminal to the fourth cysteine of the CRD is not I or T; (f) the first residue N-terminal to the fourth cysteine of the CRD is L: (g) the first residue N-terminal to the D-Xa-L motif is E and the second residue C-terminal to the D-Xa-L motif is selected from the group consisting of E, D, W, F and M; (h) the second residue C-terminal to the D-Xa-L motif is selected from the group consisting of E, D) W, F or M and the first residue N-terminal to the fourth cysteine residue of the CRD is L, (i) the first residue N-terminal to the D-Xa-L motif is E and the first residue N-terminal to the D-Xa-L motif is L; and (j) the first residue N-terminal to the D-Xa-L motif is E, the second residue C-terminal to the D-Xa-L motif is selected from the group consisting of E, D, W. F or M and the first residue N-terminal to the fourth cysteine of the CRD is L.

24-32. (canceled)

33. The polypeptide according to claim 22, wherein the polypeptide binds APRIL with an IC50 value of 500 nM or less, 100 nM or less, 50 nM or less, 10 nM or less, 5 nM or less or 1 nM or less.

34. A variant TACI polypeptide, wherein the polypeptide comprises a sequence wherein residues 94-99 of human TACI (SEQ ID NO: 10) are present at residues 55-61 of human TACI (SEQ ID NO:10).

35-39. (canceled)

40. A polypeptide comprising an altered TACI CRD2 sequence, wherein the polypeptide has increased specificity for BAFF over APRIL as compared to a naturally occurring TACI CRD2 sequence.

41. The polypeptide according to claim 40, wherein the altered TACI CRD2 sequence differs from a naturally occurring CRD2 sequence at least by a residue substitution at the second residue N-terminal to the D-Xa-L motif.

42. The polypeptide according to claim 41, wherein the residue at the second residue N-terminal to the D-Xa-L motif is not F or Y.

43. The polypeptide according to claim 40, wherein the altered TACI CRD2 sequence differs from a naturally occurring CRD2 sequence at least by a residue substitution at the first residue N-terminal to the D-Xa-L motif.

44. The polypeptide according to claim 43, wherein the residue at the first residue N-terminal to the D-Xa-L motif is not F, W or Y.

45. The polypeptide according to claim 40, wherein the altered TACI CRD2 sequence differs from a naturally occurring CRD2 sequence at least by a residue substitution at the second residue N-terminal to the D-Xa-L motif and a residue substitution at the first residue N-terminal to the D-Xa-L motif.

46. The polypeptide according to claim 41, wherein the altered TACI CRD2 sequence comprises E or S as the second residue N-terminal to the D-Xa-L motif.

47. The polypeptide according to claim 43, wherein the altered TACI CRD2 sequence comprises V as the first residue N-terminal to the D-Xa-L motif.

48. The polypeptide according to claim 45, wherein the altered TACI CRD2 sequence comprises E or S as the second residue N-terminal to the D-Xa-L motif and a V as the first residue N-terminal to the D-Xa-L motif.

49. A polypeptide comprising an altered TACI CRD2 sequence, wherein the polypeptide has increased specificity for APRIL over BAFF as compared to a naturally occurring TACI CRD2 sequence.

50. The polypeptide according to claim 49, wherein the altered TACI CRD2 sequence differs from a naturally occurring CRD2 sequence at least by a residue substitution at the first residue N-terminal to the D-Xa-L motif.

51. The polypeptide according to claim 49, wherein the residue at the first residue N-terminal to the D-Xa-L motif is not F, W or Y.

52. The polypeptide according to claim 49, wherein the altered TACI CRD2 sequence differs from a naturally occurring CRD2 sequence at least by a residue substitution at the second residue C-terminal to the D-Xa-L motif.

53. The polypeptide according to claim 49, wherein the residue at the second residue C-terminal to the D-Xa-L motif is not G, H or R.

54. The polypeptide according to claim 49, wherein the altered TACI CRD2 sequence differs from a naturally occurring CRD2 sequence at least by a residue substitution at the first residue N-terminal to the fourth cysteine of the CRD.

55. The polypeptide according to claim 50, wherein the residue at the first residue N-terminal to the fourth cysteine of the CRD is not I, R or T.

56. The polypeptide according to claim 49, wherein the altered TACI CRD2 sequence differs from a naturally occurring CRD2 sequence at least by a residue substitution at the first residue N-terminal to the D-Xa-L motif and a residue substitution at the second residue C-terminal to the D-Xa-L motif.

57. The polypeptide according to claim 49, wherein the altered TACI CRD2 sequence differs from a naturally occurring CRD2 sequence at least by a residue substitution at the second residue C-terminal to the D-Xa-L motif and a residue substitution at the first residue N-terminal to the fourth cysteine of the CRD.

58. The polypeptide according to claim 49, wherein the altered TACI CRD2 sequence differs from a naturally occurring CRD2 sequence at least by a residue substitution at the first residue N-terminal to the D-Xa-L motif and a residue substitution at the first residue N-terminal to the fourth cysteine of the CRD.

59. The polypeptide according to claim 49, wherein the altered TACI CRD2 sequence differs from a naturally occurring CRD2 sequence at least by a residue substitution at the first residue N-terminal to the D-Xa-L motif, a residue substitution at the second residue C-terminal to the D-Xa-L motif and a residue substitution at the first residue N-terminal to the fourth cysteine of the CRD.

60. The polypeptide according to any one of claims 50, 56, and 59, wherein the altered TACI CRD2 sequence comprises E as the first residue N-terminal to the D-Xa-L motif.

61. The polypeptide according to any one of claims 52, 56, and 59, wherein the altered TACI CRD2 sequence comprises E, W, D, F or M as the second residue C-terminal to the D-Xa-L motif.

62. The polypeptide according to any one of claims 54, 55, 57, and 58, wherein the altered TACI CRD2 sequence comprises L as the first residue N-terminal to the fourth cysteine of the CRD.

63. The polypeptide according to claim 49 or 59, wherein the altered TACI CRD2 sequence comprises E as the first residue N-terminal to the D-Xa-L motif, E, W, D, F or M as the second residue C-terminal to the D-Xa-L motif and L as the first residue N-terminal to the fourth cysteine of the CRD.

64. A polypeptide comprising at least one of any one of the following sequences:
(SEQ ID NO: 15)
CRKEQGKEYDHLLRDCISCASICGQHPKQCAYFC,
(SEQ ID NO: 16)
CRKEQGKSYDHLLRDCISCASICGQHPKQCAYFC,
(SEQ ID NO: 17)
CRKEQGKFVDHLLRDCISCASICGQHPKQCAYFC,
(SEQ ID NO: 18)
CRKEQGKEVDHLLRDCISCASICGQHPKQCAYFC,
(SEQ ID NO: 19)
CRKEQGKSVDHLLRDCISCASICGQHPKQCAYFC,
(SEQ ID NO: 20)
CPEEQYWDPLLGTCMSCKTICGQHPKQCAAFC,
(SEQ ID NO: 21)
CPEEQEWDPLLGTCMSCKTICGQHPKQCAAFC,
(SEQ ID NO: 22)
CPEEQSWDPLLGTCMSCKTICGQHPKQCAAFC,
(SEQ ID NO: 23)
CPEEQYVDPLLGTCMSCKTICGQHPKQCAAFC,
(SEQ ID NO: 24)
CPEEQEVDPLLGTCMSCKTICGQHPKQCAAFC,
(SEQ ID NO: 25)
CPEEQSVDPLLGTCMSCKTICGQHPKQCAAFC,
(SEQ ID NO: 26)
CRKEQGKFEDHLLRDCISCASICGQHPKQCAYFC,
(SEQ ID NO: 27)
CRKEQGKFYDHLLEDCISCASICGQHPKQCAYFC,
(SEQ ID NO: 28)
CRKEQGKFYDHLLWDCISCASICGQHPKQCAYFC,
(SEQ ID NO: 29)
CRKEQGKFYDHLLDDCISCASICGQHPKQCAYFC,
(SEQ ID NO: 30)
CRKEQGKFYDHLLFDCISCASICGQHPKQCAYFC,
(SEQ ID NO: 31)
CRKEQGKFYDHLLMDCISCASICGQHPKQCAYFC,
(SEQ ID NO: 32)
CRKEQGKFYDHLLRDCISCASLCGQHPKQCAYFC,
(SEQ ID NO: 33)
CRKEQGKFEDHLLEDCISCASICGQHPKQCAYFC,
(SEQ ID NO: 34)
CRKEQGKFEDHLLWDCISCASICGQHPKQCAYFC,
(SEQ ID NO: 35)
CRKEQGKFEDHLLDDCISCASICGQHPKQCAYFC,
(SEQ ID NO: 36)
CRKEQGKFEDHLLFDCISCASICGQHPKQCAYFC,
(SEQ ID NO: 37)
CRKEQGKFEDHLLMDCISCASICGQHPKQCAYFC,
(SEQ ID NO: 38)
CRKEQGKFYDHLLEDCISCASLCGQHPKQCAYFC,
(SEQ ID NO: 39)
CRKEQGKFYDHLLWDCISCASLCGQHPKQCAYFC,
(SEQ ID NO: 40)
CRKEQGKFYDHLLDDCISCASLCGQHPKQCAYFC,
(SEQ ID NO: 41)
CRKEQGKFYDHLLFDCISCASLCGQHPKQCAYFC,
(SEQ ID NO: 42)
CRKEQGKFYDHLLMDCISCASLCGQHPKQCAYFC,
(SEQ ID NO: 43)
CRKEQGKFEDHLLEDCISCASLCGQHPKQCAYFC,
(SEQ ID NO: 44)
CRKEQGKFEDHLLWDCISCASLCGQHPKQCAYFC,
(SEQ ID NO: 45)
CRKEQGKFEDHLLDDCISCASLCGQHPKQCAYFC,
(SEQ ID NO: 46)
CRKEQGKFEDHLLFDCISCASLCGQHPKQCAYFC,
(SEQ ID NO: 47)
CRKEQGKFEDHLLMDCISCASLCGQHPKQCAYFC,
(SEQ ID NO: 48)
CPEEQYEDPLLGTCMSCKTICGQHPKQCAAFC,
(SEQ ID NO: 49)
CPEEQYWDPLLETCMSCKTICGQHPKQCAAFC,
(SEQ ID NO: 50)
CPEEQYWDPLLWTCMSCKTICGQHPKQCAAFC,
(SEQ ID NO: 51)
CPEEQYWDPLLDTCMSCKTICGQHPKQCAAFC,
(SEQ ID NO: 52)
CPEEQYWDPLLFTCMSCKTICGQHPKQCAAFC,
(SEQ ID NO: 53)
CPEEQYWDPLLMTCMSCKTICGQHPKQCAAFC,
(SEQ ID NO: 54)
CPEEQYWDPLLGTCMSCKTLCGQHPKQCAAFC,
(SEQ ID NO: 55)
CPEEQYEDPLLETCMSCKTICGQHPKQCAAFC,
(SEQ ID NO: 56)
CPEEQYEDPLLWTCMSCKTICGQHPKQCAAFC,
(SEQ ID NO: 57)
CPEEQYEDPLLDTCMSCKTICGQHPKQCAAFC,
(SEQ ID NO: 58)
CPEEQYEDPLLFTCMSCKTICGQHPKQCAAFC,
(SEQ ID NO: 59)
CPEEQYEDPLLMTCMSCKTICGQHPKQCAAFC,
(SEQ ID NO: 60)
CPEEQYWDPLLETCMSCKTLCGQHPKQCAAFC,
(SEQ ID NO: 61)
CPEEQYWDPLLWTCMSCKTLCGQHPKQCAAFC,
(SEQ ID NO: 62)
CPEEQYWDPLLDTCMSCKTLCGQHPKQCAAFC,
(SEQ ID NO: 63)
CPEEQYWDPLLFTCMSCKTLCGQHPKQCAAFC,
(SEQ ID NO: 64)
CPEEQYWDPLLMTCMSCKTLGGQHPKQCAAFC,
(SEQ ID NO: 65)
CPEEQYEDPLLETCMSCKTLCGQHPKQCAAFC,
(SEQ ID NO: 66)
CPEEQYEDPLLWTCMSCKTLCGQHPKQCAAFC,
(SEQ ID NO: 67)
CPEEQYEDPLLDTCMSCKTLCGQHPKQCAAFC,
(SEQ ID NO: 68)
CPEEQYEDPLLFTCMSCKTLCGQHPKQCAAFC,
and
(SEQ ID NO: 69)
CPEEQYEDPLLMTCMSCKTLCGQHPKQCAAFC.

65. The polypeptide according to claim 40, wherein the polypeptide binds BAFF with an IC50 value of 500 nM or less, 100 nM or less, 50 nM or less, 10 nM or less, 5 nM or less or 1 nM or less.

66. The polypeptide according to claim 49, wherein the polypeptide binds APRIL with an IC50 value of 500 nM or less, 100 nM or less, 50 nM or less, 10 nM or less, 5 nM or less or 1 nM or less.

67-69. (canceled)

70. The polypeptide according to any one of claims 1, 2, 40, 49, or 64, wherein the polypeptide further comprises a sequence heterologous to a native TACI polypeptide sequence, wherein the heterolozous sequence is an Fc region of an IgG.

71-73. (canceled)

74. The polypeptide according to any one of claims 1, 2, 40, 49, or 64, wherein the polypeptide is an immunoadhesin.

75. The polypeptide according to any one of claims 40 or 49, wherein the polypeptide is conjugated to an agent selected from the group consisting of a growth inhibitory agent, a cytotoxic agent, a detection agent, an agent that improves the bioavailability of the polypeptide, and an agent that improves the half-life of the polypeptide, and a non-proteinaceous polymer.

76-78. (canceled)

79. A nucleic acid molecule encoding the polypeptide according any one of claims 40, 49, or 64.

80. A vector comprising the nucleic acid molecule according to claim 79.

81. A host cell comprising the nucleic acid molecule according to claim 79 or a vector comprising the nucleic acid molecule.

82. A composition comprising the polypeptide according to any one of claims 40, 49, or 64, optionally further comprising a pharmaceutically acceptable carrier.

83. A composition comprising the polypeptide according to any one of claims 40, 49, or 64, optionally further comprising at least a second therapeutic agent selected from the group consisting of an agent for treating an immune-related disease, a chemotherapeutic agent and a cytotoxic agent.

84. A method for inhibiting a TACI signaling pathway or TACI biological activity in a subject, comprising administering to the subject a polypeptide of any of claims 1, 2, 40, 49, or 64, in an amount sufficient to inhibit the TACI signaling pathway or TACI biological activity.

85. A method for inhibiting a BAFF signaling pathway or BAFF biological activity in a subject, comprising administering to the subject a polypeptide of claim 40 in an amount sufficient to inhibit the BAFF signaling pathway or BAFF biological activity.

86. A method for inhibiting a BR3 signaling pathway or BR3 biological activity in a subject, comprising administering to the subject a polypeptide of claim 40 in an amount sufficient to inhibit the BR3 signaling pathway or BR3 biological activity.

87. A method for inhibiting a BCMA signaling pathway or BCMA biological activity in a subject, comprising administering to the subject a polypeptide of any of claims 40 or 49 in an amount sufficient to inhibit the BCMA signaling pathway or BCMA biological activity.

88. A method for inhibiting an APRIL signaling pathway or APRIL biological activity in a subject in need thereof, comprising administering to the subject a polypeptide of claim 49 in an amount sufficient to inhibit the APRIL signaling pathway or APRIL biological activity.

89. A method for inhibiting a BAFF-BR3, BAFF-BCMA, APRIL-BMCA, BAFF-TACI, or APRIL-TACI interaction in a subject, comprising the step of administering to the subject a polypeptide of any of claims 40 or 49 in an amount sufficient to block or partially block the interaction.

90. A method for depleting B cells from a B cell population in a subject, comprising administering to the subject a polypeptide of any of claims 40 or 49 in an amount sufficient to decrease B cell levels.

91. The method of claim 90, further comprising administering to the subject one or more additional therapeutic agents.

92. The method of claim 91, wherein the one or more additional therapeutic agent comprises an anti-CD20 therapy or a chemotherapeutic agent.

93. The method of claim 92, wherein the anti-CD20 therapy is an anti-CD20 antibody.

94. A method for treating an immune-related disease in a mammal, comprising the step of administering to the mammal a therapeutically effective amount of a polypeptide of any of claims 40 or 49.

95. The method of claim 94, wherein said immune-related disease is selected from the group consisting of rheumatoid arthritis, multiple sclerosis, Sjogren's syndrome and systemic lupus erythematosis.

96. A method for treating a cancer in a mammal, comprising the step of administering to the mammal a therapeutically effective amount of a polypeptide of any of claims 40 or 49.

97. The method of claim 96, wherein the cancer is a B cell neoplasia or a B cell malignancy.

98. The method of claim 96, wherein the cancer is selected from the group consisting of CLL, NHL, ALL, and multiple myeloma.

99. The method of claim 96, wherein said cancer is a gastrointestinal cancer or a glioblastoma.

100. A method for treating a T-cell mediated disease in a mammal, comprising the step of administering to the mammal a therapeutically effective amount of a polypeptide of any of claims 40 or 49.

101. The method of claim 100, wherein said T-cell mediated disease is selected from the group consisting of graft rejection, graft verses host disease (GVHD), and inflammation.

102. A method for treating a B-cell regulated autoimmune disorder in a mammal, comprising the step of administering to the mammal a therapeutically effective amount of a polypeptide of any of claims 40 or 49.

103. The method of claim 94, further comprising administering to the subject one or more additional therapeutic agents.

104. The method of claim 103, wherein the one or more additional therapeutic agents comprises an anti-CD20 therapy or a chemotherapeutic agent.

105. The method of claim 104, wherein the anti-CD20 therapy is an anti-CD20 antibody.

106. The method of claim 96, further comprising administering to the subject one or more additional therapeutic agents.

107. The method of claim 106, further comprising administering to the subject one or more additional therapeutic agents.

108. The method of claim 107, further comprising administering to the subject one or more additional therapeutic agents.
